(19) United States
(12) Patent Application Publication
(10) Pub. No.: US 2012/0159656 A1
(43) Pub. Date: Jun. 21, 2012

(54) COMPOSITIONS AND METHODS FOR EVALUATING COGNITIVE DEFECTS

(75) Inventors: David J. Gerber, Somerville, MA (US); Jonathan M. Levenson, Walpole, MA (US); Margaret E. Levin, Cambridge, MA (US); Arnold Heynen, Providence, RI (US)

(73) Assignee: Galenea Corp., Cambridge, MA (US)

(21) Appl. No.: 13/266,031

(22) PCT Filed: Apr. 23, 2010
(86) PCT No.: PCT/US2010/001212

§ 371 Date: Mar. 9, 2012

Related U.S. Application Data
(60) Provisional application No. 61/172,690, filed on Apr. 24, 2009.
(60) Provisional application No. 61/248,802, filed on Oct. 5, 2009.
(60) Provisional application No. 61/317,244, filed on Mar. 24, 2010.

(51) Int. Cl. A61B 5/0484; G01N 33/48

(52) U.S. Cl. 800/3; 600/544

(57) ABSTRACT

The present invention provides, in some aspects, methods for identifying agents useful in treating disorders or conditions associated with cognitive deficits. In some aspects, the invention provides methods for detecting a cognitive deficit in a subject.

### RELATED APPLICATIONS

This application claims the benefit under 35 U.S.C. §119(e) of U.S. provisional application Ser. No. 61/172,690, filed Apr. 24, 2009, U.S. provisional application Ser. No. 61/248,802, filed Oct. 5, 2009 and U.S. provisional application Ser. No. 61/317,244, filed Mar. 24, 2010, the entire contents of each of which are incorporated herein by reference.

### BACKGROUND OF THE INVENTION

Cognitive deficits in schizophrenia include deficits such as impairments in executive function, attention, and working memory. The prefrontal cortex is a brain region that is required for these higher level cognitive processes. There is a need for improved in vivo, preclinical methods for evaluating agents to treat cognitive deficits in schizophrenia, with better predictive capacity for efficacy in human patients. There is also a need for effective methods to treat cognitive deficits in schizophrenia and in related disorders.

### SUMMARY OF THE INVENTION

The invention in some aspects is based on the discovery that oscillatory components of an electroencephalographic (EEG) signal obtained from an animal, e.g., a mouse, a human, etc., provide information regarding the cognitive state of the animal. In some embodiments, characteristic EEG oscillation signatures are provided that are indicative of cognitive state. The invention encompasses the finding that specific alterations in high frequency neural activity (gamma oscillations) in the prefrontal cortex occur in multiple animal models of schizophrenia and related disorders associated with cognitive deficits. Certain aspects of the invention, are based on the discovery that characteristic EEG oscillation signatures are not limited to a particular species, but rather are observed across multiple species, including, for example, mice and humans. Thus, it has been discovered that animals having certain diseases associated with cognitive deficits exhibit characteristic alterations in oscillation signatures. In some embodiments, the EEG oscillation signatures provide a basis for identifying candidate therapeutic agents for treating cognitive deficits based on changes in the EEG oscillation signatures. In certain embodiments, methods of diagnosing and monitoring a cognitive deficit in an animal, e.g., a mouse or human, are provided.

According to some aspects of the invention, methods of identifying a candidate therapeutic agent for treatment of a cognitive deficit. The methods include (a) administering a test agent to a test animal, wherein the test animal comprises a cognitive deficit, and the cognitive deficit is characterized by a distribution of the power of gamma oscillations recorded from a brain area during the cognitive task that substantially differs from a control distribution of the power of gamma oscillations recorded from the brain area of a control animal during the cognitive task; (b) recording gamma oscillations from the brain area of the test animal while the test animal is engaged in the cognitive task; (c) determining the distribution of the power of gamma oscillations in the test animal during the cognitive task; and (d) comparing the determined distribution of the power of gamma oscillations of the test animal to the control distribution of the power of gamma oscillations, wherein a test agent that substantially reduces a difference between the distribution of the power of gamma oscillations in the test animal compared to the control distribution, is identified as a candidate therapeutic agent for treatment of the cognitive deficit. In some embodiments, the gamma oscillations are Gamma_{Hi} oscillations. In certain embodiments, the Gamma_{Hi} oscillations are in a range of 65 Hz to 90 Hz. In some embodiments, the cognitive deficit is associated with schizophrenia. In some embodiments, the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety. In some embodiments, the control distribution is a bimodal distribution. In certain embodiments, the test animal is a rodent. In some embodiments, the rodent is a rat or mouse. In some embodiments, the test animal is a primate. In some embodiments, the primate is a non-human primate. In certain embodiments, the primate is a human. In some embodiments, the animal has a neurological disorder or condition or is a non-human animal model of such neurological disorder or condition. In some embodiments, the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety. In some embodiments, the neurological disorder or condition or non-human animal model of such neurological disorder or condition is chemically induced. In some embodiments, the neurological disorder or condition or non-human animal model of such neurological disorder or condition is chemically induced with a drug that impairs glutamatergic function in the animal. In certain embodiments, the drug is selected from: phencyclidine (PCP), MK-801, and ketamine. In some embodiments, the neurological disorder or condition or non-human animal model of such neurological disorder or condition is chemically induced with a drug that enhances dopaminergic function in the animal. In some embodiments, the drug is selected from: apomorphine, D-amphetamine, and methamphetamine. In some embodiments, the neurological disorder or condition or non-human animal model of such neurological disorder or condition is chemically induced with a hallucinogenic drug. In some embodiments, the hallucinogenic drug is selected from: mescaline, lysergic acid diethylamide (LSD), and psilocybin. In certain embodiments, the neurological disorder or condition or non-human animal model of such neurological disorder or condition is chemically induced with a drug that impair cholinergic function. In some embodiments, the drug is scopolamine. In some embodiments, the neurological disorder or condition or non-human animal model of such neurological disorder or condition is a genetically induced. In some embodiments, the animal is a calcineurin knock-out mouse (CNKO mouse). In certain embodiments, calcineurin is knocked-out postnatally in forebrain neurons of the animal. In some embodiments, the cognitive task is a novel object recognition task, a Delayed Non-Match-To-Position task, an alternating T-Maze, a Set Shifting task, an 8-arm radial maze task, 5 choice serial reaction time test, or an odor spanning task. In some embodiments, the task is a novel oddball task. In some embodiments, the cognitive task utilizes both attention and executive function of the animal. In certain embodiments, the brain area is the prefrontal cortex, the striatum, the hippocampus, a midbrain dopaminergic area, In some embodiments, the midbrain dopaminergic area is ventral tegmental area. In some embodiments, recording gamma oscillations in (b) comprises recording a single-unit activity (SUA) from the brain area. In some embodiments, recording gamma oscillations in (b) comprises recording an electrophysiological signal from an implanted electrode. In certain embodiments, recording gamma oscillations in (b) comprises recording from a brain area comprising the frontal association cortex. In some embodiments, the animal is a mouse and the gamma oscillations are recorded from a region of brain that is within medial-lateral extent posterior to the olfactory bulb, anterior to M2 motor cortex, and superficial to orbital cortex. In some embodiments, the animal is a mouse and recording gamma oscillations comprises recording from a brain area having the coordinates: from Bregma +0.37 cm rostral, +0.07 cm lateral, −0.05 cm deep from the brain surface. In some embodiments, recording gamma oscillations in (b) comprises recording an electrophysiological signal from an external electrode. In certain embodiments, the external electrode is a scalp electrode. In some embodiments, the candidate therapeutic agent is a bimodal modulator of gamma oscillation.

According to other aspects of the invention, methods of identifying a candidate therapeutic agent for treatment of a cognitive deficit are provided. The methods include (a) administering a test agent to a test animal, wherein the test animal is an animal comprising a cognitive deficit, and the cognitive deficit is characterized by a distribution of the power of electroencephalographic oscillations recorded from a brain area during a cognitive task that substantially differs from a control distribution of the power of electroencephalographic oscillations recorded from the brain area of a control animal during the cognitive task; (b) recording electroencephalographic oscillations from the brain area of the test animal while the test animal is engaged in the cognitive task; (c) determining the distribution of the power of electroencephalographic oscillations in the test animal during the cognitive task; and (d) comparing the determined distribution of the power of electroencephalographic oscillations of the test animal to the control distribution of the power of electroencephalographic oscillations, wherein a test agent that substantially reduces a difference between the distribution of the power of electroencephalographic oscillations in the test animal compared to the control distribution, is identified as a candidate therapeutic agent for treatment of the cognitive deficit. In some embodiments, the electroencephalographic oscillations are gamma oscillations. In some embodiments, the gamma oscillations are Gamma_{Low} oscillations. In certain embodiments, the gamma oscillations are Gamma_{Hi} oscillations. In some embodiments, the gamma oscillations are in a range of 30 Hz to 90 Hz. In some embodiments, the Gamma_{Low} oscillations are in a range of 30 Hz to 55 Hz. In some embodiments, the Gamma_{Hi} oscillations are in a range of 65 Hz to 90 Hz. In some embodiments, the electroencephalographic oscillations are gamma oscillations that have an average power when recorded from a control animal exposed to a novel environment that is substantially higher than the average power when recorded from a control animal exposed to a familiar environment. In certain embodiments, the electroencephalographic oscillations are gamma oscillations that have an average power when recorded from a calcineurin knock out animal exposed to a novel environment that is substantially equal to the average power when recorded from a control animal exposed to a familiar environment. In some embodiments, the electroencephalographic oscillations are theta oscillations or ripple oscillations. In some embodiments, the theta oscillations are in a range of 4 Hz to 12 Hz. In certain embodiments, the ripple oscillations are in a range of 100 Hz to 300 Hz. In some embodiments, the cognitive deficit is associated with schizophrenia. In some embodiments, the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety. In some embodiments, the control distribution is a bimodal distribution. In certain embodiments, the test animal is a rodent. In some embodiments, the rodent is a rat or mouse. In some embodiments, the test animal is a primate. In some embodiments, the primate is a non-human primate. In certain embodiments, the primate is a human. In some embodiments, the animal has a neurological disorder or condition or is a non-human animal model of such neurological disorder or condition. In some embodiments, the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety. In some embodiments, the neurological disorder or condition or non-human animal model of such neurological disorder or condition is chemically induced. In certain embodiments, the neurological disorder or condition or non-human animal model of such neurological disorder or condition is chemically induced with a drug that impairs glutamatergic function in the animal. In some embodiments, the drug is selected from: phencyclidine (PCP), MK-801, and ketamine. In some embodiments, the neurological disorder or condition or non-human animal model of such neurological disorder or condition is chemically induced with a drug that enhances dopaminergic function in the animal. In some embodiments, the drug is selected from: apomorphine, D-amphetamine, and methamphetamine. In certain embodiments, the neurological disorder or condition or non-human animal model of such neurological disorder or condition is chemically induced with a hallucinogenic drug. In some embodiments, the hallucinogenic drug is selected from: mescaline, lysergic acid diethylamide (LSD), and psilocybin. In some embodiments, the neurological disorder or condition or non-human animal model of such neurological disorder or condition is chemically induced with a drug that impairs cholinergic function. In some embodiments, the drug is scopolamine. In some embodiments, the neurological disorder or condition or non-human animal model of such neurological disorder or condition is genetically induced. In certain embodiments, the animal is a calcineurin knock-out mouse (CNKO mouse). In some embodiments, calcineurin is knocked-out postnatally in forebrain neurons of the animal. In some embodiments, the cognitive task is a novel object recognition task. In some embodiments, the cognitive task is a Delayed Non-Match-To-Position task, an alternating T-Maze, a Set Shifting task, an 8-arm radial maze task, 5 choice serial reaction time test, or an odor spanning task. In certain embodiments, the task is a novelty oddball task. In some embodiments, the cognitive task utilizes both attention and executive function of the animal. In some embodiments, the brain area is the prefrontal cortex. In some embodiments, the brain area is the striatum. In some embodiments, the brain area is the hippocampus. In certain embodiments, the brain area is a midbrain dopaminergic area. In some embodiments, the midbrain dopaminergic area is ventral tegmental area. In some embodiments, recording electroencephalographic oscillations/activity in (b) comprises high-pass filtering and recording a single-unit activity (SUA) from the brain area. In some embodiments, recording electroencephalographic oscillations in (b) comprises recording an electrophysiological signal from an implanted electrode. In some embodiments, recording electroencephalographic oscillations in (b) comprises recording from a brain area comprising the frontal association cortex. In certain embodiments, the electroencephalographic oscillations are recorded from a region of brain that is within medial-lateral extent posterior to the olfactory bulb, anterior to M2 motor cortex, and superficial to orbital cortex. In some embodiments, the animal is a mouse and recording electroencephalographic oscillations comprises recording from a brain area having the coordinates: from Bregma +0.37 cm rostral, +0.07 cm lateral, −0.05 cm deep from the brain surface. In some embodiments, recording electroencephalographic oscillations in (b) comprises recording an electrophysiological signal from an external electrode. In certain embodiments, the external electrode is a scalp electrode. In some embodiments, the candidate therapeutic agent is a bimodal modulator of gamma oscillation.

According to other aspects of the invention, methods of identifying a candidate therapeutic agent for treatment of a cognitive deficit are provided. The methods include (a) administering a test agent to a test animal, wherein the test animal is an animal comprising a cognitive deficit, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task; (b) recording an electroencephalographic oscillation from the brain area of the test animal while the test animal is engaged in the cognitive task; and (c) comparing, in the predetermined frequency range, the recorded electroencephalographic oscillation of the test animal to the control electroencephalographic oscillation, wherein a test agent that substantially reduces a difference between the electroencephalographic oscillation in the test animal compared to the control electroencephalographic oscillation, is identified as a candidate therapeutic agent for treatment of the cognitive deficit. In some embodiments, comparing in (c) includes comparing power determined in the predetermined frequency range of the electroencephalographic oscillation of the test animal to power in the predetermined frequency range of the control electroencephalographic oscillation. In some embodiments, comparing in (c) includes comparing a distribution of powers of the electroencephalographic oscillation to a distribution of powers of the control electroencephalographic oscillation. In some embodiments, comparing in (c) includes comparing a frequency histogram of powers determined in predetermined time intervals of the electroencephalographic oscillation to a frequency histogram of powers determined in predetermined time intervals of the control electroencephalographic oscillation. In certain embodiments, the predetermined frequency range is 30 Hz to 90 Hz. In some embodiments, the predetermined frequency range is 65 Hz to 90 Hz. In some embodiments, the predetermined frequency range is 30 Hz to 55 Hz. In some embodiments, the predetermined frequency range is a frequency range of a theta oscillation or a frequency range of a ripple oscillation. In some embodiments, the predetermined frequency range is a frequency range within which the electroencephalographic oscillation has an average power when recorded from a control animal exposed to a novel environment that is substantially higher than the average power when recorded from a control animal exposed to a familiar environment. In certain embodiments, the predetermined frequency range is a frequency range within which the electroencephalographic oscillation has an average power when recorded from a calcineurin knock out animal exposed to a novel environment that is substantially equal to the average power when recorded from a control animal exposed to a familiar environment. In some embodiments, the cognitive deficit is associated with schizophrenia. In some embodiments, the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety. In some embodiments, the control distribution is a bimodal distribution. In certain embodiments, the test animal is a rodent. In some embodiments, the rodent is a rat or mouse. In some embodiments, the test animal is a primate. In some embodiments, the primate is a non-human primate. In certain embodiments, the primate is a human. In some embodiments, the animal has a neurological disorder or condition or is a non-human animal model of such neurological disorder or condition. In some embodiments, the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety. In certain embodiments, the neurological disorder or condition or non-human animal model of such neurological disorder or condition is chemically induced. In some embodiments, the neurological disorder or condition or non-human animal model of such neurological disorder or condition is chemically induced with a drug that impairs glutamatergic function in the animal. In some embodiments, the drug is selected from: phencyclidine (PCP), MK-801, and ketamine. In some embodiments, the neurological disorder or condition or non-human animal model of such neurological disorder or condition is chemically induced with a drug that enhances dopaminergic function in the animal. In certain embodiments, the drug is selected from: apomorphine, D-amphetamine, and methamphetamine. In some embodiments, the neurological disorder or condition or non-human animal model of such neurological disorder or condition is chemically induced with a hallucinogenic drug. In some embodiments, the hallucinogenic drug is selected from: mescaline, lysergic acid diethylamide (LSD), and psilocybin. In some embodiments, the neurological disorder or condition or non-human animal model of such neurological disorder or condition is chemically induced with a drug that impair cholinergic function. In certain embodiments, the drug is scopolamine. In some embodiments, the neurological disorder or condition or non-human animal model of such neurological disorder or condition is genetically induced. In some embodiments, the animal is a calcineurin knock-out mouse (CNKO mouse). In some embodiments, calcineurin is knocked-out postnatally in forebrain neurons of the animal. In certain embodiments, the cognitive task is a novel object recognition task, a Delayed Non-Match-To-Position task, an alternating T-Maze, a Set Shifting task, an 8-arm radial maze task, 5 choice serial reaction time test, or an odor spanning task. In some embodiments, the task is a novelty oddball task. In some embodiments, the cognitive task utilizes both attention and executive function of the animal. In some embodiments, the brain area is the prefrontal cortex. In certain embodiments, the brain area is the striatum. In some embodiments, the brain area is the hippocampus. In some embodiments, the brain area is a midbrain dopaminergic area. In some embodiments, the midbrain dopaminergic area is ventral tegmental area. In certain embodiments, recording electroencephalographic oscillations/activity in (b) comprises high-pass filtering and recording single-unit activity (SUA) from the brain area. In some embodiments, recording electroencephalographic oscillations in (b) comprises recording an electrophysiological signal from an implanted electrode. In some embodiments, recording electroencephalographic oscillations in (b) comprises recording from a brain area comprising the frontal association cortex. In some embodiments, the electroencephalographic oscillations are recorded from a region of brain that is within medial-lateral extent posterior to the olfactory bulb, anterior to M2 motor cortex, and superficial to orbital cortex. In certain embodiments, the animal is a mouse and the recording electroencephalographic oscillations comprises recording from brain area having the coordinates: from Bregma +0.37 cm rostral, +0.07 cm lateral, −0.05 cm deep from the brain surface. In some embodiments, recording electroencephalographic oscillations in (b) comprises recording an electrophysiological signal from an external electrode. In some embodiments, the external electrode is a scalp electrode. In some embodiments, the candidate therapeutic agent is a bimodal modulator of gamma oscillation.

According to other aspects of the invention, methods of detecting a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, is provided. The methods including (a) recording an electroencephalographic oscillation from the brain area of the animal while the animal is engaged in a cognitive task; and (b) comparing, in the predetermined frequency range, the electroencephalographic oscillation recorded in (a) of the animal to the control electroencephalographic oscillation, wherein a substantial difference between the electroencephalographic oscillation in the animal compared to the control electroencephalographic oscillation, indicates that the animal has a cognitive deficit. In certain embodiments, a substantial difference between the electroencephalographic oscillation in the animal compared to the control electroencephalographic oscillation is detected and the method further comprises diagnosing the animal as having the cognitive deficit. In some embodiments, the methods also include (c) determining a distribution of the power of gamma oscillations in the electroencephalographic oscillation recorded in (a); and (d) obtaining a control distribution of the power of gamma oscillations in the control electroencephalographic oscillation, wherein comparing in (b) comprises comparing, in the predetermined frequency range, the distribution of the power of gamma oscillations in the electroencephalographic oscillation determined in (c) to the distribution of the power of gamma oscillations in the control electroencephalographic oscillation obtained in (d), wherein a substantial difference between the distribution of the power of gamma oscillations in the electroencephalographic oscillation determined in (c) compared to the distribution of the power of gamma oscillations in the control electroencephalographic oscillation obtained in (d), indicates that the animal has the cognitive deficit. In some embodiments, a substantial difference between the distribution of the power of gamma oscillations in the electroencephalographic oscillation determined in (c) compared to the distribution of the power of gamma oscillations in the control electroencephalographic oscillation obtained in (d) is detected and the method further comprises diagnosing the animal as having the cognitive deficit.

According to other aspects of the invention, methods of monitoring a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, are provided. The methods including (a) recording an electroencephalographic oscillation from the brain area of the animal while the animal is engaged in the cognitive task; (b) comparing, in the predetermined frequency range, the electroencephalographic oscillation recorded in (a) of the animal to the control electroencephalographic oscillation, wherein a substantial difference between the electroencephalographic oscillation in the animal compared to the control electroencephalographic oscillation, indicates that the animal has a cognitive deficit; and (c) repeating steps (a) and (b) one or more times, thereby monitoring the cognitive deficit in the animal. In some embodiments, the methods also include: (d) administering a treatment for the cognitive disorder to the animal before (c), and (e) comparing the electroencephalographic oscillation recorded in the animal before the treatment to the electroencephalographic oscillation recorded in the animal after the treatment to monitor the efficacy of the treatment.

According to other aspects of the invention, methods of monitoring the effect of a treatment on a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, are provided. The methods including (a) recording an electroencephalographic oscillation from the brain area of the animal with a cognitive deficit while the animal is engaged in the cognitive task; (b) determining a distribution of the power of gamma oscillations in the electroencephalographic oscillation recorded in the animal; (c) administering a treatment for the cognitive deficit or for a disease associated with the cognitive deficit to the animal with the cognitive impairment; (d) recording an electroencephalographic oscillation from the brain area of the treated animal while the animal is engaged in the cognitive task; (e) determining a distribution of the power of gamma oscillations in the electroencephalographic oscillation recorded in the treated animal; and (f) comparing the distribution of power in (b) to the distribution of power in (e), wherein a substantial difference in the power in (b) and the power in (e) indicates an effect of the treatment on the cognitive deficit in the animal, and wherein a distribution of power in (e) that is more similar to a normal control distribution of power than is the distribution of power in (b), indicates efficacy of the treatment.

According to other aspects of the invention, methods of determining the efficacy of a treatment for a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, are provided. The methods including (a) recording an electroencephalographic oscillation from the brain area of the animal while the animal is engaged in the cognitive task; (b) determining a distribution of the power of gamma oscillations in the electroencephalographic oscillation recorded in (a) of the animal; (c) comparing, in the predetermined frequency range, the distribution of the power of gamma oscillations determined in (b) to a control distribution of the power of gamma oscillations, wherein a substantial difference between the distribution of the power of gamma oscillations in the animal compared to the control distribution, indicates that the animal has a cognitive deficit; (d) administering a treatment for the cognitive deficit to the animal; and (e) repeating steps (a) to (c) one or more times after administering the treatment in step (d), wherein a substantial decrease in a difference between the distribution of the power of gamma oscillations in the animal compared to the control distribution, indicates that the treatment is effective for treating the cognitive deficit.

In some embodiments of any of the aforementioned methods of the invention, the treatment is a precognitive agent, an antipsychotic, antidepressant, anti-dementia, antiepileptic or anti-anxiety medication. In certain embodiments of any of the aforementioned methods of the invention, the predetermined frequency range is 30 Hz to 90 Hz. In some embodiments of any of the aforementioned methods of the invention, the predetermined frequency range is 65 Hz to 90 Hz. In some embodiments of any of the aforementioned methods of the invention, the predetermined frequency range is 30 Hz to 55 Hz. In some embodiments of any of the aforementioned methods of the invention, the predetermined frequency range is a frequency range of a theta oscillation or a frequency range of a ripple oscillation. In some embodiments of any of the aforementioned methods of the invention, the predetermined frequency range is a frequency range within which the electroencephalographic oscillation has an average power when recorded from a control animal exposed to a novel environment that is substantially higher than the average power when recorded from a control animal exposed to a familiar environment. In certain embodiments of any of the aforementioned methods of the invention, the predetermined frequency range is a frequency range within which the electroencephalographic oscillation has an average power when recorded from a calcineurin knock out animal exposed to a novel environment that is substantially equal to the average power when recorded from a control animal exposed to a familiar environment. In some embodiments of any of the aforementioned methods of the invention, the cognitive deficit is associated with schizophrenia. In some embodiments of any of the aforementioned methods of the invention, the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety. In some embodiments of any of the aforementioned methods of the invention, the control distribution is a bimodal distribution. In some embodiments of any of the aforementioned methods of the invention, the animal is a rodent. In certain embodiments, the rodent is a rat or mouse. In some embodiments of any of the aforementioned methods of the invention, the animal is a primate. In some embodiments of any of the aforementioned methods of the invention, the primate is a non-human primate. In some embodiments of any of the aforementioned methods of the invention, the primate is a human. In some embodiments of any of the aforementioned methods of the invention, the animal has a neurological disorder or condition or is a non-human animal model of such neurological disorder or condition. In certain embodiments of any of the aforementioned methods of the invention, the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety. In some embodiments of any of the aforementioned methods of the invention, the neurological disorder or condition or non-human animal model of such neurological disorder or condition is chemically induced. In some embodiments of any of the aforementioned methods of the invention, the neurological disorder or condition or non-human animal model of such neurological disorder or condition is chemically induced with a drug that impairs glutamatergic function in the animal. In some embodiments of any of the aforementioned methods of the invention, the drug is selected from: phencyclidine (PCP), MK-801, and ketamine. In some embodiments of any of the aforementioned methods of the invention, the neurological disorder or condition or non-human animal model of such neurological disorder or condition is chemically induced with a drug that enhances dopaminergic function in the animal. In certain embodiments of any of the aforementioned methods of the invention, the drug is selected from: apomorphine, D-amphetamine, and methamphetamine. In some embodiments of any of the aforementioned methods of the invention, the neurological disorder or condition or non-human animal model of such neurological disorder or condition is chemically induced with a hallucinogenic drug. In some embodiments of any of the aforementioned methods of the invention, the hallucinogenic drug is selected from: mescaline, lysergic acid diethylamide (LSD), and psilocybin. In some embodiments of any of the aforementioned methods of the invention, the neurological disorder or condition or non-human animal model of such neurological disorder or condition is chemically induced with a drug that impairs cholinergic function. In certain embodiments of any of the aforementioned methods of the invention, the drug is scopolamine. In some embodiments of any of the aforementioned methods of the invention, the neurological disorder or condition or non-human animal model of such neurological disorder or condition is genetically induced. In some embodiments of any of the aforementioned methods of the invention, the animal is a calcineurin knock-out mouse (CNKO mouse). In some embodiments of any of the aforementioned methods of the invention, calcineurin is knocked-out postnatally in forebrain neurons of the animal. In certain embodiments of any of the aforementioned methods of the invention, the cognitive task is a novel object recognition task, a Delayed Non-Match-To-Position task, an alternating T-Maze, a Set Shifting task, an 8-arm radial maze task, 5 choice serial reaction time test, or an odor spanning task. In some embodiments of any of the aforementioned methods of the invention, the cognitive task is a Novelty Oddball task. In some embodiments of any of the aforementioned methods of the invention, the cognitive task utilizes both attention and executive function of the animal. In some embodiments of any of the aforementioned methods of the invention, the brain area is the prefrontal cortex. In certain embodiments of any of the aforementioned methods of the invention, the brain area is the striatum. In some embodiments of any of the aforementioned methods of the invention, the brain area is the hippocampus. In some embodiments of any of the aforementioned methods of the invention, the brain area is a midbrain dopaminergic area. In some embodiments of any of the aforementioned methods of the invention, the midbrain dopaminergic area is ventral tegmental area. In certain embodiments of any of the aforementioned methods of the invention, recording electroencephalographic oscillations/activity in (b) comprises high-pass filtering and recording single-unit activity (SUA) from the brain area. In some embodiments of any of the aforementioned methods of the invention, recording electroencephalographic oscillations in (b) comprises recording an electrophysiological signal from an implanted electrode. In some embodiments of any of the aforementioned methods of the invention, recording electroencephalographic oscillations in (b) comprises recording from a brain area comprising the frontal association cortex. In some embodiments of any of the aforementioned methods of the invention, the electroencephalographic oscillations are recorded from a region of brain that is within medial-lateral extent posterior to the olfactory bulb, anterior to M2 motor cortex, and superficial to orbital cortex. In certain embodiments of any of the aforementioned methods of the invention, wherein the animal is a mouse and the recording electroencephalographic oscillations comprises recording from brain area having the coordinates: from Bregma +0.37 cm rostral, +0.07 cm lateral, −0.05 cm deep from the brain surface. In some embodiments of any of the aforementioned methods of the invention, recording electroencephalographic oscillations in (b) comprises recording an electrophysiological signal from an external electrode. In some embodiments of any of the aforementioned methods of the invention, the external electrode is a scalp electrode.

According to other aspects of the invention, methods of determining an effect of a candidate agent on a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task are provided. The methods including (a) recording an electroencephalographic oscillation from the brain area of the animal with a cognitive deficit while the animal is engaged in the cognitive task; (b) determining a distribution of the power of gamma oscillations in the electroencephalographic oscillation recorded in the animal; (c) administering the candidate agent to the animal with the cognitive impairment; (d) recording an electroencephalographic oscillation from the brain area of the treated animal while the animal is engaged in the cognitive task; (e) determining a distribution of the power of gamma oscillations in the electroencephalographic oscillation recorded in the treated animal; and (f) comparing the distribution of power in (b) to the distribution of power in (e), wherein a substantial difference in the distribution of power in (b) and the distribution of power in (e) indicates an effect of the candidate agent on the cognitive deficit in the animal. In some embodiments, the candidate agent is a precognitive agent, an antipsychotic, antidepressant, anti-dementia, antiepileptic or anti-anxiety medication. In certain embodiments, the candidate agent is a small molecule. In some embodiments, the predetermined frequency range is 30 Hz to 90 Hz, 65 Hz to 90 Hz, or 30 Hz to 55 Hz. In some embodiments, the method is utilized in a clinical trial. In some embodiments, the distribution of the power of gamma oscillations is utilized as a biomarker in a clinical trial. In certain embodiments, the predetermined frequency range is a frequency range of a theta oscillation or a frequency range of a ripple oscillation. In some embodiments, the predetermined frequency range is a frequency range within which the electroencephalographic oscillation has an average power when recorded from a control animal exposed to a novel environment that is substantially higher than the average power when recorded from a control animal exposed to a familiar environment. In some embodiments, the cognitive deficit is associated with schizophrenia. In some embodiments, the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety. In certain embodiments, the control distribution is a bimodal distribution. In some embodiments, the animal is a rodent. In some embodiments, the rodent is a rat or mouse. In certain embodiments, the animal is a primate. In some embodiments, the primate is a non-human primate. In some embodiments, the primate is a human. In some embodiments, the animal has a neurological disorder or condition or is anon-human animal model of such neurological disorder or condition. In certain embodiments, the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety. In some embodiments, the neurological disorder or condition or non-human animal model of such neurological disorder or condition is chemically induced. In some embodiments, the neurological disorder or condition or non-human animal model of such neurological disorder or condition is chemically induced with a drug that impairs glutamatergic function in the animal. In some embodiments, the drug is selected from: phencyclidine (PCP), MK-801, and ketamine. In certain embodiments, the neurological disorder or condition or non-human animal model of such neurological disorder or condition is chemically induced with a drug that enhances dopaminergic function in the animal. In some embodiments, the drug is selected from: apomorphine, D-amphetamine, and methamphetamine. In some embodiments, the neurological disorder or condition or non-human animal model of such neurological disorder or condition is chemically induced with a hallucinogenic drug. In certain embodiments, the hallucinogenic drug is selected from: mescaline, lysergic acid diethylamide (LSD), and psilocybin. In some embodiments, the neurological disorder or condition or non-human animal model of such neurological disorder or condition is chemically induced with a drug that impairs cholinergic function. In some embodiments, the drug is scopolamine. In some embodiments, the neurological disorder or condition or non-human animal model of such neurological disorder or condition is genetically induced. In certain embodiments, the animal is a calcineurin knock-out mouse (CNKO mouse). In some embodiments, calcineurin is knocked-out postnatally in forebrain neurons of the animal. In some embodiments, the cognitive task is a novel object recognition task, a Delayed Non-Match-To-Position task, an alternating T-Maze, a Set Shifting task, an 8-arm radial maze task, 5 choice serial reaction time test, or an odor spanning task, or a Novelty Oddball task. In some embodiments, the cognitive task utilizes both attention and executive function of the animal. In certain embodiments, the brain area is the prefrontal cortex. In some embodiments, the brain area is the striatum. In some embodiments, the brain area is the hippocampus. In certain embodiments, the brain area is a midbrain dopaminergic area. In some embodiments, the midbrain dopaminergic area is ventral tegmental area. In some embodiments, recording electroencephalographic oscillations/activity in (b) comprises high-pass filtering and recording single-unit activity (SUA) from the brain area. In some embodiments, recording electroencephalographic oscillations in (b) comprises recording an electrophysiological signal from an implanted electrode. In certain embodiments, recording electroencephalographic oscillations in (b) comprises recording from a brain area comprising the frontal association cortex. In some embodiments, recording electroencephalographic oscillations in (b) comprises recording an electrophysiological signal from an external electrode. In certain embodiments, the external electrode is a scalp electrode.

According to some aspects of the invention, methods are provided that comprise steps of: administering to an animal having a neurological disorder or condition such as psychosis or a cognitive impairment a candidate therapeutic agent; recording gamma oscillations from the PFC of the animal; determining the distribution of the power of gamma oscillations in the animal; evaluating if the agent restores the distribution of gamma oscillations to a bimodal distribution corresponding to cognitive status. In some embodiments, the methods further comprise a step of identifying the agent as a bimodal modulator of gamma oscillation power based on the evaluation result form step (d). In some embodiments, the methods further comprise a step of testing the ability of the identified bimodal modulator to treat a psychosis or cognitive deficit. In some embodiments, the identified bimodal modulator is tested for its ability to treat a cognitive deficit associated with schizophrenia. In some embodiments, the identified bimodal modulator is tested for its ability to treat a cognitive deficit associated with bipolar disorder, Alzheimer's disease, Parkinson's disease, Attention Deficit Hyperactivity Disorder (ADHD), multiple sclerosis, autism, or anxiety. In some embodiments, the animal is a rodent. In some embodiments, the animal is a mouse. In some embodiments, the animal is a rat. In some embodiments, the animal is a pharmacological model of a neurological disorder or condition. In some embodiments, the neurological disorder or condition is induced by administration of a glutamatergic agent. In some embodiments, the glutamatergic agent is PCP, MK-801 or ketamine. In some embodiments, the neurological disorder or condition is induced by a dopaminergic agent. In some embodiments, the dopaminergic agent is amphetamine or cocaine. In some embodiments, the neurological disorder or condition is induced by administration of a dopaminergic agent. In some embodiments, the dopaminergic agent is amphetamine or cocaine. In some embodiments, the neurological disorder or condition is a genetic neurological disorder or condition. In some embodiments, the animal is a calcineurin heterozygous knockout mouse. In some embodiments, the recordings are performed while the animal is performing a behavioral task. In some embodiments, the task is novel object recognition. In some embodiments, the task is Delayed Non-Match-To-Position. In some embodiments, the task is set shifting. In some embodiments, the task is a radial arm maze. In some embodiments, the task is a T maze or Y maze. In some embodiments, the task is an odor span task.

According to some aspects of the invention, methods are provided that comprise: administering to an individual who is suffering from or susceptible to psychosis an effective amount of an agent that is a bimodal modulator of gamma oscillation power, such that at least one symptom or feature of the psychosis is reduced in intensity, severity, or frequency, or has delayed onset. According to some aspects of the invention, methods are provided that comprise: administering to an individual who is suffering from or susceptible to a cognitive deficit an effective amount of an agent that is a bimodal modulator of gamma oscillation power, such that at least one symptom or feature of the cognitive deficit is reduced in intensity, severity, or frequency, or has delayed onset. In some embodiments, the cognitive deficit is associated with schizophrenia. In some embodiments, the cognitive deficit is associated with bipolar disorder, Alzheimer's disease, Parkinson's disease, Attention Deficit Hyperactivity Disorder (ADHD), multiple sclerosis, autism, or anxiety.

### BRIEF DESCRIPTION OF THE DRAWINGS

FIG. 1 depicts exemplary frequency histograms (distribution) of the power of gamma oscillations recorded from the prefrontal cortex (PFC) of normal mice, calcineurin knockout mice and heterozygous calcineurin knock-out mice exposed to a novel environment. Using this methodology, normal mice exhibit a bimodal distribution of “High” and “Low” power gamma oscillations. Calcineurin knockout mice exhibit a unimodal distribution of “Low” power gamma oscillations, and heterozygous calcineurin knockout mice exhibit a unimodal distribution of “Intermediate” power gamma oscillations.

FIG. 2 exhibits an exemplary recording trace and frequency histogram of the power of gamma oscillations recorded from prefrontal cortex of normal mice treated with PCP (5 mg/kg) in a familiar environment. Using this methodology, PCP administration resulted in a unimodal distribution of gamma oscillations with “Intermediate” power similar to that observed in the heterozygous calcineurin knockout mouse model.

FIG. 3 shows a schematic diagram of exemplary gamma oscillation signatures observed in disease states and normal states at baseline and under situations where cognitive engagement occurs in the normal state. Desired patterns of gamma power restoration that would be achieved with effective therapeutic agents in the baseline state and states of higher cognitive engagement are shown.

FIG. 4 shows electroencephalogram (EEG) traces recorded from prefrontal cortex of human and mouse. Shown are representative examples of EEG recordings from human and mouse prefrontal cortex (PFC). Raw traces are band-pass filtered to reveal specific frequency components. Human and mouse waveform morphology and frequency are similar across all frequency ranges. Human traces were adapted from publicly available recordings. Mouse traces were made from a female C57Bl/6 mouse.

FIG. 5 shows development of EEG and single-unity activity (SUA) recording technique in mouse PFC. A series of surgical and electrophysiological techniques have been developed for recording EEGs from the PFC of freely behaving mice. FIG. 5A provides a diagram of the surgical procedure. An electrode consisting of a bundle of 8 tungsten microwires is stereotaxically placed in PFC of mouse. A silver ground wire is placed directly above cerebellum. Connection to the computer monitoring EEGs is done via an Omnetics connector. FIG. 5B shows a representative brain from a mouse that underwent surgical implantation of a microwire bundle electrode in the PFC. Dashed circle indicates position of electrode track. Sagittal section from brain depicted on left stained with cresyl-violet reveals electrode track in the PFC. C) FIG. 5C shows a frame of a movie depicting a freely behaving mouse in an operant chamber. Below the movie are real-time recordings from one of the electrodes in the bundle. Shown are the raw EEG trace and three traces that have been bandpass filtered (Theta: 4-9 Hz, Gamma: 30-90 Hz, Ripple: 100-300 Hz). FIG. 5D shows a spectral analysis of EEGs of 3 mice exposed to a novel environment for 30 min that revealed a bimodal distribution of gamma power in the PFC (F_{[3,486]}=18, p<0.001). Note the lower power peak at 2 μV²/Hz (indicated by solid arrow) and a higher power peak at 5 μV²/Hz (indicated by open arrow).

FIG. 6 shows high power gamma oscillations in PFC that are associated with attention. Recordings from PFC were obtained from freely behaving animals well habituated to the recording chamber. FIG. 6A shows baseline measurements of PFC EEGs that were made in well-habituated animals in the absence of novel environmental stimuli. Average power of gamma oscillations was 2 μV²/Hz. FIG. 6B shows measurements of PFC EEGs made after addition of novel objects to the environment that revealed a significant number of episodes of high-power gamma activity (8 μV²/Hz). These episodes of high-power gamma oscillations were coincident with behavior directed toward the novel objects. Representative EEGs shown above summary histograms were band-pass filtered to reveal gamma oscillations (30-90 Hz). Summary histograms indicate relative number of episodes of gamma activity as a function of power.

FIG. 7 shows significant impairment in a Delayed Non-Match-To-Position (DNMTP) task in calcineurin knockout animals. Wild type (FF) and forebrain-specific calcineurin knockout animals (FFCRE) were trained in a DNMTP task. Delay intervals indicate latency to begin nonmatch trial from nosepoke. Notice that FFCRE animals exhibit a profound deficit in their ability to perform this task (F_{[1,188]}=304, p<0.0001).

FIG. 8 shows loss of high power gamma oscillations in forebrain-specific calcineurin knockout mice. EEGs were recorded from PFC of wild type (F/F) and forebrain-specific calcineurin knockout animals (F/F-CRE) in a novel environment. EEGs from F/F animals exhibited a robust, bimodal distribution of low (indicated by solid arrow) and high-power (indicated by open arrow) gamma oscillations (data re-plotted from FIG. 2D). In contrast, EEGs recorded from F/F-CRE animals exhibited a unimodal distribution of low-power gamma oscillations (F_{[2, 1,300]}=5, p<0.01). Representative EEG traces are shown above summary data. High-power gamma oscillations were markedly absent in the F/F-CRE trace. Calibration bars apply to both traces.

FIG. 9 shows a loss of high power gamma oscillations measured in acute brain slices containing the PFC in calcineurin knockout mice. Sagittal sections (400 μm) were made from brains derived from either wild type (F/F) or CNKO (F/F-CRE) animals. FIG. 9A shows an image of a brain slice on the perforated multielectrode array (MEA). Solid black disks are electrodes and black lines from each disk are leads. Spacing of electrodes is 200 μm. Indicated on this particular image are positions of PFC, motor cortex (M2) and Prelimbic (PrL) cortex. FIG. 9B shows summary data indicating a significant reduction in power of evoked gamma oscillations in PFC from F/F-CRE animals relative to F/F littermates (F_{[1,13]}=15.41, P<0.005). Horizontal bar in summary data indicates perfusion of carbachol (20 μM) into the slice chamber. Representative band-pass filtered traces depicting gamma oscillations before and during the addition of carbachol are shown above summary data for both F/F and F/F-CRE animals (scale bars apply to all traces).

FIG. 10 shows a loss of high power gamma oscillations in the PFC after administration of PCP. Animals that were habituated to the recording chamber were exposed to objects before and after intraperitoneal administration of PCP (5 mg/kg). EEGs from PFC were recorded continuously. FIG. 10A shows recordings made before injection of PCP in the absence and presence of novel objects. Note the prominent high-power gamma event peak (open arrow) during object presentation (8 μV²/Hz; F_{[3,150]}=19, p<0.0001). FIG. 10B shows recordings made after injection of PCP in the absence and presence of novel objects. Note the lack of a bimodal distribution in gamma oscillation power (F_{[3,150]}=0.3, p<0.9), indicating PCP inhibits the ability of PFC to express high-power gamma oscillations during periods of object exploration. Pictures illustrate images from continuous video monitoring during experiment. Representative traces show bandpass-filtered recordings (30-90 Hz) before and during object presentation. Scale bars apply to all traces.

FIG. 11 shows an analysis of EEGs in the Gamma_{Hi} frequency band (65-90 Hz) recorded from PFC of mice. EEGs were recorded from the PFC of mice and analyzed using the methods described in Example 7, focusing on the Gamma_{Hi} frequency band. In FIG. 11A, EEGs recorded in a novel environment revealed significant gene dosage-dependent effects on average power observed in the Gamma_{Hi} frequency band (F_{[2,79]}=766, p <0.0001; control: n=16; CNₕₑₜKO: n=20; CNKO: n=4). FIG. 11B shows that when recorded in a familiar environment, EEGs from the PFC of CNₕₑₜKO (n=21) exhibit significantly higher power in the Gamma_{Hi} frequency band relative to EEGs recorded from CNKO (t=8, p<0.001, n=3) or littermate control animals (t=38, p<0.001, n=17). In FIG. 11C, treatment of control animals with PCP (n=5) increased the average spectral power in the Gamma_{Hi} frequency band of the EEG in the PFC (F_{[2,28]}=603, p<0.0001). Notably, the average power observed in control animals treated with PCP in a familiar environment (19.12±0.11, n=5) was similar to the power observed in CNₕₑₜKO animals (18.42±0.05, n=21; dashed line). Values reported for n in this figure also apply to FIGS. 12-14. Post-hoc t-statistics were performed using the method of Bonferroni.

FIG. 12 shows an analysis of EEGs in the Gamma_{Low} frequency band (30-55 Hz) recorded from PFC of mice. EEGs were recorded from the PFC of mice and analyzed as described in Example 7, focusing on the Gamma_{Low} frequency band (30-55 Hz). In FIG. 12A, EEGs recorded in a novel environment revealed that CNKO animals exhibit more high-powered events compared to CNₕₑₜKO animals (t=3, p<0.01). In FIG. 12B, EEGs recorded in a familiar environment revealed that CNKO animals exhibit more high-powered events compared to CNₙₑₜKO (t=8, p<0.001) and littermate control animals (t=5, p<0.001). In FIG. 12C, treatment of control animals with PCP increased the average spectral power in the Gamma_{Low} frequency band of the EEG in the PFC (t=21, df=8, p<0.0001)

FIG. 13 shows an analysis of EEGs in a Ripple frequency band (100-300 Hz) recorded from PFC of mice. EEGs were recorded from the PFC of mice and analyzed as described in Example 7, focusing on the Ripple frequency band (100-300 Hz). In FIG. 13A, EEGs recorded in a novel environment revealed a significant decrease in EEG power in the Ripple frequency band recorded from CNKO animals relative to both CNₕₑₜKO (t=9, p<0.001) and littermate controls (t=7, p<0.001). FIG. 13B shows that no differences between genotypes were observed in the Ripple frequency band EEG power recorded from animals in a familiar environment. In FIG. 13C, treatment of control animals with PCP had no effect on the average spectral power in the Ripple frequency band of the EEG in the PFC.

FIG. 14 shows an analysis of EEGs in a Theta frequency band (4-12 Hz) recorded from PFC of mice. EEGs were recorded from the PFC of mice and analyzed as described in Example 7, focusing on the Theta frequency band (4-12 Hz). In FIG. 14A, EEGs recorded in a novel environment revealed a significant decrease in Theta frequency band EEG power in a gene dosage-dependent fashion (F_{[2,112]}=145, p<0.0001). Control animals exhibited the highest theta power; CNKO animals exhibited the lowest with CNₙₑₜKO animals exhibiting an intermediate power. In FIG. 14B, no differences between genotypes were observed in EEG power when recorded from animals in a familiar environment. In FIG. 14C, treatment of control animals with PCP increased the average spectral power in the theta frequency band of the EEG in the PFC (t=10, df=8, p<0.0001).

FIG. 15 depicts bimodality of power distributions as determined using an analysis of ensemble EEG power in a predetermined frequency band across two behavioral states according to the methods described in Example 7. EEG power distributions are depicted for novel and familiar environments, and a combination of the two distributions produces a bimodal distribution. In a familiar environment, predominantly low power events are observed whereas in a novel environment predominantly higher power events are observed. When considering the entirety of these conditions, a bimodal distribution similar to the one observed using the methods of Example 1 was observed.

FIG. 16 depicts spectral power in the Gamma wide band (30-90 Hz) that was measured in animals in a novel environment using method described in Example 7. Control animals exhibited a bimodal distribution of power, with a sharp peak in the low power range and a broad peak in the high power range, consistent with our observations using the method of Example 1. Both heterozygous and homozygous knockout mice exhibit overlapping, low power peaks. F/F and F/+ are wild type control mice; F/+-CRE are heterozygous calcineurin knock-out (CNₙₑₜKO) mice; and F/F-CRE are homozygous calcineurin knock-out (CNKO) mice.

FIG. 17A provides a schematic diagram illustrating an exemplary method of detecting a cognitive deficit in a test animal. FIG. 17B provides a schematic diagram illustrating an exemplary method of determining a distribution of power of EEG oscillation using spectral analysis.

FIG. 18 provides results from an exemplary Power Spectral Analysis. FIG. 18A shows a representative power spectrum from control (F/+) animal during a period of attending behavior. Gamma_{Low} and Gamma_{Hi} frequency bands are denoted by dashed lines. A prominent peak in the Gamma_{Hi} band is noted (arrow), which represents significant network activity. FIG. 18B depicts an input/output curve generated from an EEG electrode. I/O curve was generated by playing a 1-500 Hz chirp stimulus and performing a power spectral analysis. The signal decays by 1/Frequency. Inset shows signal decay across the full Gamma spectrum.

FIG. 19A shows human EEG time-frequency clusters that revealed several frequency bands which were regulated in response to visual novelty oddball stimuli. A robust increase in power is observed in the Gamma_{Hi} band in frontal cortex (‘Cluster 1’), which preceded changes in the Gamma_{Low} frequency band (Clusters 2-4). The low-frequency cluster (‘Cluster 5’) represents the ERP, a synchronous EEG phenomenon associated with acute exposure to oddball sensory stimuli. FIG. 19B shows a summary of time-frequency analyses of Novel-Dim data using the windowed periodogram analysis employed for the analysis of rodent EEG data. Increases in Gamma_{Hi} power were observed in both Fp1 and Fp2 (corresponding to the left and right frontal cortex, respectively). Calculating the difference between Fp1 and Fp2 revealed a robust increase in Gamma_{Hi} power approximately 700 msec after novelty oddball exposure.

### DETAILED DESCRIPTION OF CERTAIN EMBODIMENTS OF THE INVENTION

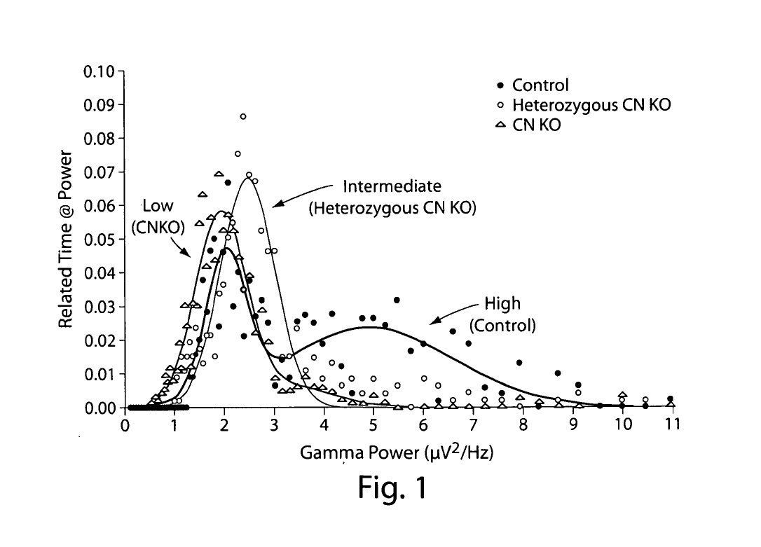

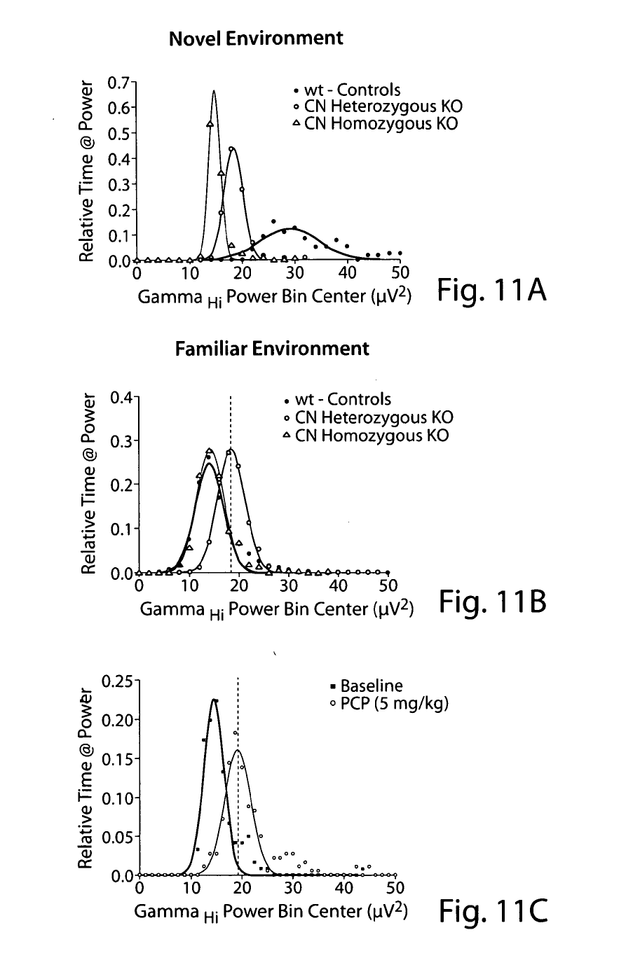

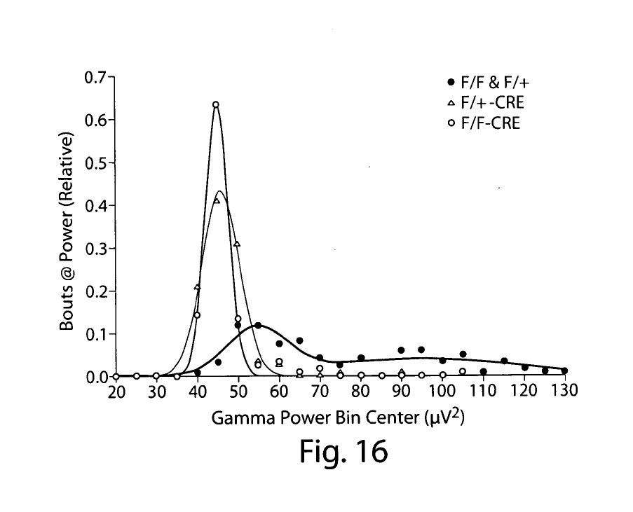

The invention, in some aspects, is based on the discovery that oscillatory components of an electroencephalographic (EEG) signal obtained from an animal provide information regarding the cognitive state of the animal and the information can be used, in part, in methods to identify agents that can alter the cognitive state of the animal. A characteristic EEG oscillation signature has been discovered that is indicative of behavioral state and that provides a basis for identifying agents that influence behavioral state. EEG oscillation signatures manifest as distinct patterns in statistical distributions of EEG-derived power. Different behavioral states are induced in animals by exposing the animals to a novel environment and/or to a familiar environment. Statistical distributions of ensemble EEG power within the gamma frequency band determined from the animals exhibit distinct modes that correspond with the animals' behavioral state (See, e.g., FIG. 11 and FIG. 16). Similarly, when maximum EEG powers within the gamma frequency band are evaluated, a bimodal distribution of power is observed, with each mode of the distribution corresponding to a behavioral state (See, e.g., FIG. 1). Typically, for normal, non-cognitively impaired animals a high power mode is observed that corresponds with exposure to the novel environment and a low power mode is observed that corresponds with exposure to a familiar environment. Similar oscillatory signatures have now been observed in other frequency bands of the EEG signal, including, for example, the theta and ripple frequency bands.

It has also been discovered that animals having certain diseases associated with cognitive deficits (e.g., schizophrenia, etc.) exhibit characteristic alterations in oscillation signatures. For example, when ensemble EEG power within the gamma frequency band is evaluated for behavioral states in a calcineurin-knock out animal, an animal model of schizophrenia that exhibits multiple abnormal behaviors related to schizophrenia, only a single mode is observed, with the high power mode not observed when the animal is placed in a novel environment. Similar changes in oscillation signature are observed in animals treated with drugs that induce cognitive deficits. Certain aspects of the invention provide methods for identifying candidate therapeutic agents for treating cognitive deficits based on changes in EEG oscillation signatures.

Aspects of the invention also provide in vivo screening methods for identifying and characterizing candidate therapeutic agents for treatment of cognitive deficits. In some embodiments, cognitive deficits are characterized in an animal by evaluating electroencephalographic oscillations obtained from the animal while the animal is engaged in a cognitive task. In other aspects of the invention, test agents are assayed for their ability to improve an animal's performance in a cognitive task. The screening methods typically involve evaluating the effects of a test or candidate agent on an animal by assessing spectral powers of electroencephalographic oscillations in the gamma frequency range obtained from the animal. In some embodiments, spectral powers of electroencephalographic oscillations are assessed in the Gamma_{Hi} frequency range, which corresponds with the upper portion of the gamma frequency range.

### Methods for Assessing Cognitive Deficits

In some aspects of the invention, methods are provided for evaluating a cognitive deficit in an animal based on electroencephalographic oscillations recorded from the animal. The term “cognitive deficit”, as used herein, refers to a deficiency in cognitive ability or performance of an animal. Cognitive deficits may be caused by genetic factors, congenital factors or environmental factors, such as drug use, sleep deprivation, certain sensory inputs (e.g., excessive sound or excessive light), brain injuries, infection, disease, neurological disorders, and mental illness, among others. A cognitive deficit may be assessed, or identified, in an animal by comparing the animal's cognitive ability or performance, or an aspect thereof, with a reference [e.g., with the cognitive ability or performance of a normal animal (e.g., a normal control animal)]. In some cases, a cognitive deficit may be assessed or identified in an animal by comparing the animal's cognitive ability or performance, or an aspect thereof, with the cognitive ability or performance of the animal at an earlier point in time (e.g., at a point in a time before a traumatic injury or the onset of a disease or infection). In other cases, a cognitive deficit may be induced in an animal by treating the animal with a drug that impairs cognition (e.g., alcohol, apomorphine, d-amphetamine, methamphetamine phencyclidine (PCP), MK-801, ketamine, mescaline, lysergic acid diethylamide (LSD), psilocybin, scopolamine). The induced cognitive deficit may be identified by comparing the animal's cognitive ability or performance, or an aspect thereof, after administration of the drug that impairs cognition with the cognitive ability or performance of the animal before administration of the drug that impairs cognition.

Examples of a cognitive deficit that may be evaluated in the methods include, but are not limited to, a cognitive deficit that is associated with schizophrenia, bipolar disorder, Alzheimer's disease, Parkinson's disease, Huntington's Disease, multiple sclerosis, Attention Deficit Hyperactivity Disorder (ADHD), autism, a learning or memory disorder, a brain injury, or anxiety. In some embodiments, a cognitive deficit is psychosis or a cognitive impairment, such as, but not limited to an impairment of attention, memory, learning, speed of learning or acquisition of data, etc. In some embodiments, an animal may have one or more cognitive deficits.

As used herein, the terms “animal” and “subject” may refer to animals such as rodents, cats, dogs, birds, horses, primates and any other suitable animal. In some embodiments, a rodent is a rat or a mouse. In some embodiments, a primate is a non-human primate and in some embodiments, a primate is a human. As used herein, the term “test animal” refers to an animal that is administered a test agent in an in vivo assay. Typically, the in vivo assay is designed to evaluate a test agent's suitability as a candidate therapeutic agent for the treatment of a cognitive deficit or a neurological disorder or condition associated with a cognitive deficit. The test animal may be a normal animal (e.g., wild-type animal) or a genetically altered animal (e.g., a knock-out animal, a knock-in animal, a transgenic animal) or a surgically altered animal, or a chemically altered animal, or a behaviorally altered animal (e.g., a sleep-deprived animal). The test animal may be an inbred strain of an animal having a particular disease phenotype. Typically, when an animal has a characteristic phenotype (e.g., a disease, a surgical induced brain damage, a neurological disorder or condition) and/or known genotype (e.g., a mutation associated with disease), the animal is referred to as an “animal model” of the phenotype and/or known genotype. Thus, a test animal exhibiting one or more symptoms of a disease, neurological disorder, or condition, may be referred to herein as an “animal model of a disease”. For example, a test animal exhibiting one or more symptoms of a cognitive deficit is referred to herein as an “animal model of a cognitive deficit”.

In some cases, an animal model may be a chemically induced animal model, and a neurological disorder or condition may be a chemically induced neurological disorder or condition. For example, the animal model or neurological disorder or condition may be chemically induced with a drug that impairs glutamatergic function and mimics a psychotic disease state in the animal. Non-limiting examples of drugs that impair glutamatergic function include phencyclidine (PCP), MK-801, and ketamine. An animal model or neurological disorder or condition may be chemically induced with a drug that enhances dopaminergic function and mimics a psychotic disease state in the animal. Non-limiting examples of drugs that enhance dopaminergic function include apomorphine, D-amphetamine, and methamphetamine. An animal model or neurological disorder or condition may be chemically induced with a hallucinogenic drug that mimics positive symptoms associated with schizophrenia. Non-limiting examples of hallucinogenic drugs include mescaline, lysergic acid diethylamide (LSD), and psilocybin. An animal model or neurological disorder or condition may be chemically induced with a drug that impairs cholinergic function, which is believed to mimic aspects of the cognitive symptoms associated with schizophrenia. Non-limiting examples of drugs that impair cholinergic function include scopolamine.

Aspects of the methods involve comparing an animal's cognitive ability or performance with that of a control animal. As used herein, the term “control animal” refers to an animal having a known cognitive status. An example of a control animal, though not intended to be limiting, is an animal that is a normal, non-cognitively impaired animal. Thus in some embodiments, an agent that results in a test animal's distribution of power of electroencephalographic oscillation being more like that of a “normal” control animal, may be a candidate for treating the cognitive deficit. In other aspects of the invention, a control animal may be an animal that has the cognitive deficit of the test animal, but to which the test agent is not administered. Thus, in some embodiments of the invention, an agent that when administered to a test animal with a cognitive deficit, results in the test animal's electroencephalographic oscillations (e.g., gamma oscillations) becoming less similar to those of an untreated control that has the cognitive deficit, may be identified as a modulator of electroencephalographic oscillations (e.g., gamma oscillations). Such an agent may be a candidate therapy for treating the cognitive deficit. A test animal may also serve as its own control. For example, the cognitive ability or performance of a test animal may be compared with the animal's cognitive ability or performance at a different point in time, e.g., prior to administration of a drug, prior to the onset of disease, etc.

### Testing

Typically, the cognitive ability or performance of an animal may be assessed by evaluating the animal's response to a cognitive task. The term “cognitive task”, as used herein, refers to a task that stimulates an animal (e.g., a normal animal, test animal, etc.) to engage in cognition or an aspect of cognition. For example, a cognitive task may be a task that stimulates an animal to engage in a process of categorizing, judging, learning, perceiving, problem-solving, reasoning, recognizing, or remembering. Non-limited examples of cognitive tasks include, but are not limited to, novel object recognition, Delayed Non-Match-To-Position, 5 choice serial reaction time test, alternating T-maze, Set Shifting, 8-arm radial maze, odor spanning tasks. A cognitive task may involve exposing an animal to a novel environment. When the cognitive task involves exposing an animal to a novel environment, it is often useful to evaluate the animal's response to the novel environment by a comparison with the animal's response to a familiar environment. The skilled artisan will appreciate that the cognitive tasks disclosed herein are not intended to be limiting and that other cognitive tasks may appropriately be used with the methods disclosed herein. In testing an animal, a cognitive task used for the test and control determinations may be the same cognitive task and in some embodiments the test and control tasks may be different cognitive tasks.

### Electroencephalographic Oscillations

Certain cognitive deficits may be characterized by distinct signatures in electrophysiological signals recorded from the brain of an animal. Electrophysiological signals that are recorded from the brain are referred to herein as “electroencephalographic oscillations” and may be equivalently referred to herein as “EEG oscillations,” “electroencephalographic signals,” “electrophysiological brain signals,” or “EEG signals.” Typically, an electroencephalographic oscillation is recorded as a time-dependent voltage between a pair of electrodes positioned on, or in proximity to, brain tissue and recorded over a discrete period of time. These electroencephalographic signals may be acquired by an electroencephalogram (“EEG”) device, e.g., a system that can measure an electrical activity in the brain via one or more pairs of electrodes coupled to an animal's scalp or implanted in the animal's brain tissue.

Some aspects of the invention include stereotaxic implantation of microwire bundle electrodes into the prefrontal cortex (PFC) of animals. The location of the implantation may be in a region of brain that is within medial-lateral extent posterior to the olfactory bulb, anterior to M2 motor cortex, and superficial to orbital cortex. Exemplary, but non-limiting implantation coordinates in mice include: from Bregma: +0.37 cm rostral, +0.07 cm lateral, and −0.05 cm deep from brain surface. Following implantation, and after a recovery period for the animal, EEG traces from PFC can be recorded from the freely behaving animal in a novel environment and in a familiar environment, or similarly in any appropriate cognitive task. Such recordings can be done, for example, in an operant chamber or other chamber in which the animals can behave freely while recording is performed.

The invention, in some aspects, provides methods for recording electroencephalographic oscillations in a PFC region of an animal engaged in a cognitive task. In some embodiments, single unit activity (SUA) may be recorded from an implanted electrode. In some embodiments, recording may be done using scalp electrodes or other non-invasive recording electrodes or devices. As provided herein, recording of electroencephalographic oscillations may be done after the animal has been administered a test agent and an assessment of the ability of the agent to modulate the electroencephalographic oscillation in the animal may be compared to electroencephalographic oscillations of a control animal or test animal prior to administration of the agent, to identify whether the agent modulates electroencephalographic oscillations. Some aspects of the invention include methods of recording oscillations such as gamma oscillations (e.g., Gamma_{Hi} oscillations).

Electroencephalographic oscillations may be processed (e.g., band-pass filtered, etc.) to obtain a component oscillation having a desired (predetermined) frequency (e.g., a frequency in a range of 30 Hz to 90 Hz, a frequency in a range of 65 Hz to 90 Hz, etc.). For example, to quantify gamma oscillations, recordings of electroencephalographic oscillations may be band-pass filtered to obtain oscillations having a frequency range of 30 Hz to 90 Hz, 30 Hz to 55 Hz, or 65 Hz to 90 Hz, etc. In some embodiments, electroencephalographic oscillations are up to 1 Hz, 5 Hz, 10 Hz, 20 Hz, 30 Hz, 40 Hz, 50 Hz, 60 Hz, 70 Hz, 80 Hz, 90 Hz, 100 Hz, 150 Hz, 200 Hz, 250 Hz, 300 Hz, 350 Hz, 400 Hz, 450 Hz, 500 Hz, 750 Hz, 1000 Hz, 1500 Hz or more Hz including all values in between. In some embodiments, electroencephalographic oscillations are in a range of about 1 Hz to 5 Hz, 5 Hz to 10 Hz, 10 Hz to 20 Hz, 20 Hz to 30 Hz, 30 Hz to 40 Hz, 40 Hz to 50 Hz, 50 Hz to 60 Hz, 60 Hz to 70 Hz, 70 Hz to 80 Hz, 80 Hz to 90 Hz, 90 Hz to 100 Hz, 100 Hz to 150 Hz, 150 Hz to 200 Hz, 200 Hz to 250 Hz, 250 Hz to 300 Hz, 300 Hz to 350 Hz, 350 Hz to 400 Hz, 400 Hz to 450 Hz, 450 Hz to 500 Hz, 500 Hz to 750 Hz, 750 Hz to 1000 Hz, or 1000 Hz to 1500 Hz. In some embodiments, electroencephalographic oscillations are theta oscillations, gamma oscillations, or ripple oscillations. Theta oscillations may have a frequency range of 4 Hz to 12 Hz or 4 Hz to 9 Hz. Gamma oscillations may have a range of 30 Hz to 90 Hz. Ripple oscillations may have a range of 100 Hz to 300 Hz.

In some embodiments, the electroencephalographic oscillations are gamma oscillations that have an average power when recorded from a control animal exposed to a novel environment that is substantially higher than the average power when recorded from a control animal exposed a familiar environment. In some embodiments, the electroencephalographic oscillations are gamma oscillations that have an average power when recorded from a calcineurin knock-out animal exposed to a novel environment that is substantially equal to the average power when recorded from a control animal exposed to a familiar environment. In some embodiments, the gamma oscillations have a frequency in the upper portion (e.g., upper half) of a frequency range of 30 Hz to 90 Hz, such gamma oscillations are referred to herein as Gamma_{Hi}; oscillations. An example of a frequency range comprising Gamma_{Hi}; oscillations is 65 Hz to 90 Hz. In other embodiments, the gamma oscillations have a frequency in the lower portion (e.g., the lower half) of a frequency range of 30 Hz to 90 Hz, such gamma oscillations are referred to herein as Gamma_{Low} oscillations. An example of a frequency range comprising Gamma_{Low} oscillations is 30 Hz to 55 Hz.

It will be understood that an electroencephalographic oscillation, or a component oscillation obtained therefrom, may be represented in any one of a variety of ways. For example, the electroencephalographic oscillation, or a component oscillation obtained therefrom, may be represented in a time domain, e.g., as a voltage time series or as a power time series. The electroencephalographic oscillation may also be represented in a frequency domain, e.g., by transforming a signal from a time domain to a frequency domain (e.g., using Fast-Fourier Transform, Wavelet Transform, etc.). It will also be understood by one of ordinary skill in the art that a recording of an electroencephalographic oscillation may be processed in any one of a variety of ways to quantify different oscillatory components of the signal.

Electroencephalographic oscillations, or component oscillations obtained therefrom, may be represented as the frequency of occurrence of power (or voltage) levels in the oscillation. The frequency of occurrence of power levels in an electroencephalographic oscillation may be referred to herein as a “distribution of the power of electroencephalographic (EEG) oscillation”. As used herein, the phrase “distribution of the power of gamma oscillations”, refers to a statistical distribution of power levels measured from gamma oscillations. The power levels may be determined from the electroencephalographic oscillation by any one of a variety of methods known in the art. Typically, the power levels are determined by processing the electroencephalographic oscillations using a spectral analysis. Spectral analysis methods that may be applied in conjunction with methods disclosed herein for use to analyze electrophysiological oscillations are well known in the art (See, e.g., Van Vugt M. K. et al., Comparison of Spectral Analysis Methods for Characterizing Brain Oscillations, Journal of Neuroscience Methods, (2007) 162:49-63; Klimesch W. et al., Episodic and semantic memory; an analysis in the EEG theta band, Electroencephalogr Clin Neurophysiol 1994; 91:428-41; Whittington M. A. et al., Inhibition-based rhythms: experimental and mathematical observations on network dynamics, Int J Psychophysiol, (2000) 38:315-336; Spencer K. M. et al., Sensory-evoked gamma oscillations in chronic schizophrenia, Biol Psychiatry, (2008) 63:744-747; the contents of which relating to spectral analysis of electroencephalographic signals are incorporated herein by reference).

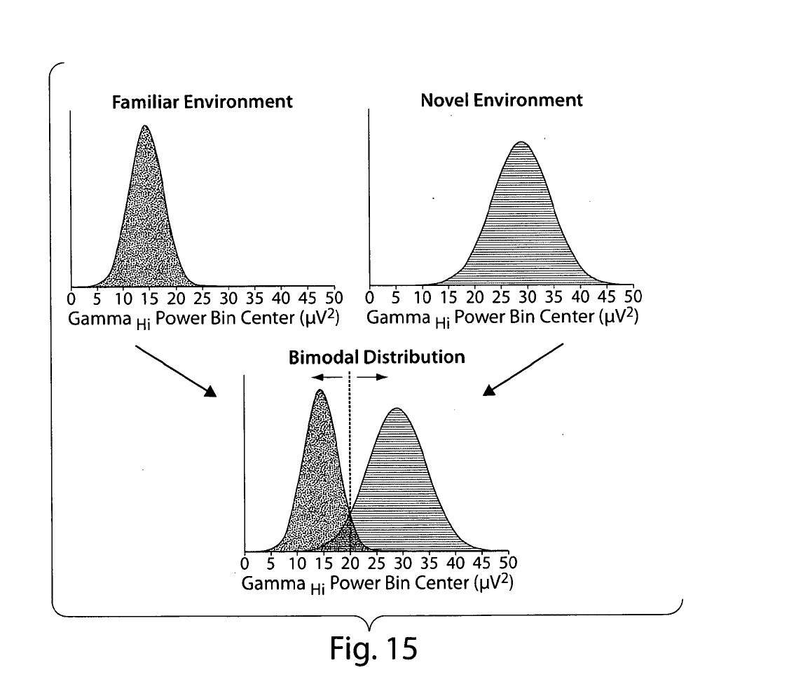

Exemplary distributions of the power of electroencephalographic oscillations, although not intended to be limiting, are depicted in FIGS. 1 and 15. In some embodiments of the invention, the distribution of power is computed by taking data from individual animals obtained over a continuous recording session and performing power analyses on consecutive time segments. As will be appreciated by the skilled artisan, data may be binned into any of a variety of time segments, for example, 5 sec., 10 sec., 15 sec., 20 sec., 25 sec., 30 sec. segments (including all times in between), and analyzed. A relative frequency histogram (a distribution) may be constructed from binning powers determined for each time segment over an entire recording session. A non-limiting example of computing the distribution of power includes taking data from individual animals obtained over a continuous, 30 minute recording session and performing power analyses on consecutive, 10 second segments. A relative frequency histogram is constructed from binning the ensemble powers for each 10 second segment over an entire recording session. Alternative time periods for recording sessions and binned segments may be used in methods of the invention.

### Exemplary Methods for Detecting a Cognitive Deficit in an Animal

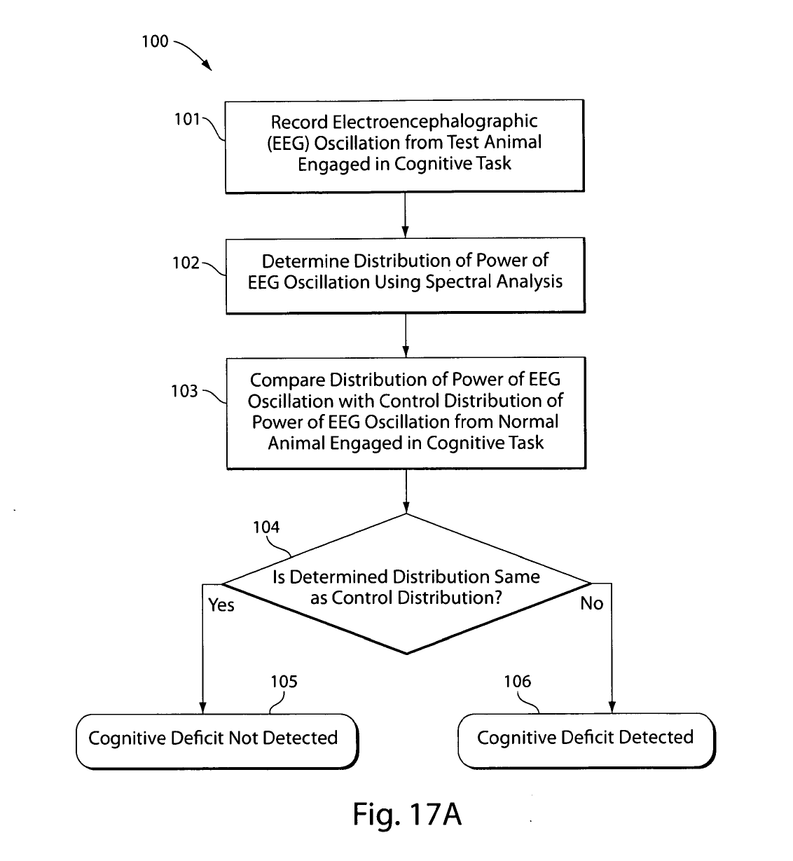

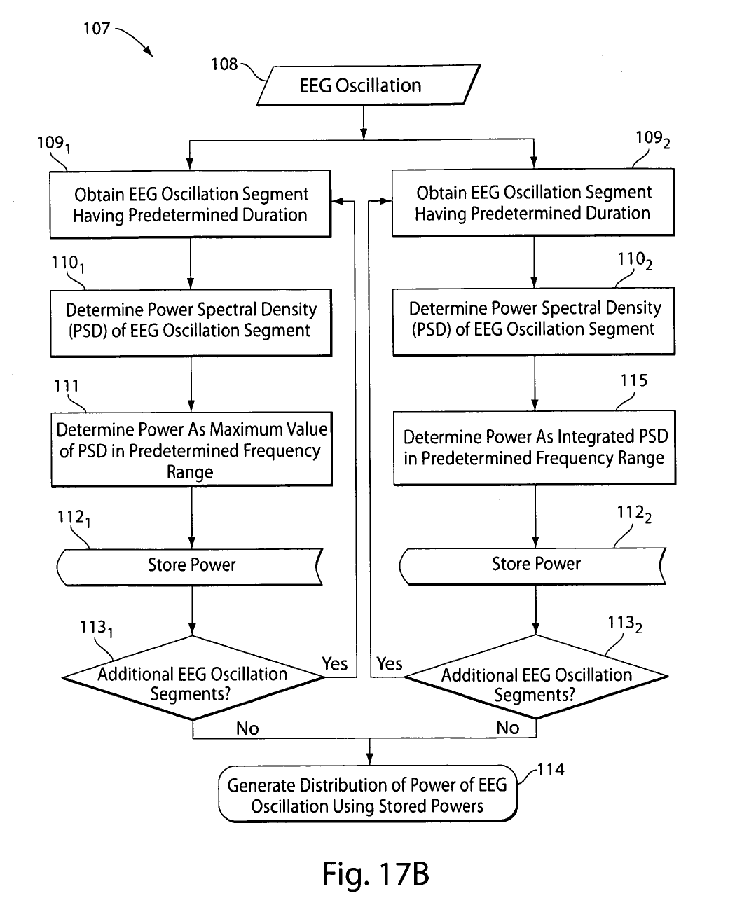

FIG. 17A illustrates an exemplary method of detecting a cognitive deficit in a test animal, 100 based on a distribution of the power of electroencephalographic oscillation. At block 101, an EEG oscillation is recorded from a test animal engaged in a cognitive task. The EEG oscillation may be recorded, for example, using an implanted electrode, or an implanted bundle of electrodes. External electrodes (e.g., scalp electrodes) or other non-invasive electrodes, may also be used to obtain an EEG oscillation from the test animal. At block 102, the distribution of power of the EEG oscillation is determined using spectral analysis. As will be appreciated by the skilled artisan, a variety of different spectral analyses may be used to determine the powers of the distribution. At block 103, the distribution of power of EEG oscillations obtained at block 102 is compared with a control distribution of power of EEG oscillation from a normal animal (an animal that does not have a cognitive deficit) engaged in a cognitive task. The method branches at decision block 104, where, if a substantial difference is identified between the distribution determined at block 102 and the control distribution (i.e., if the distribution determined at block 102 is not substantially equal to the control distribution), then a cognitive deficit is detected. If at decision block 104, a substantial difference is not identified between the distribution determined at block 102 and the control distribution (i.e., if the distribution determined at block 102 is substantially equal to the control distribution) then a cognitive deficit is not detected.

In this exemplary method of detecting a cognitive deficit in a test animal 100, a variety of cognitive tasks may be used. As will be appreciated by the skilled artisan, the cognitive task used will typically be a cognitive task that produces an EEG oscillation from which a distribution of power may be determined that is suitable for identifying a difference indicative of a cognitive deficit when compared with the control distribution of power of EEG oscillation. Suitable cognitive tasks will be apparent to the skilled artisan and examples of such tasks are disclosed herein. For example, the test animal may be placed in a novel environment and permitted to explore the novel environment for a period of time (e.g., 30 minutes) while electrodes record the EEG oscillation (e.g., a gamma, theta, ripple, etc. oscillation.) The test animal may be presented with a novel object and permitted to explore the novel object for a period of time while electrodes record the EEG oscillation. And, typically, the control distribution of power of EEG oscillation from the control animal (the normal animal) is based on, or representative of, the EEG oscillation obtained from a control animal engaged in an equivalent cognitive task as the test animal.

The comparison in block 103 may be made by any suitable method. Examples of suitable methods are disclosed herein. Typically, the distribution obtained in 102 is compared directly with the control distribution using a suitable statistical test for comparing two distributions. The comparison typically involves determining if the distribution obtained in 102 is substantially different or substantially equal to the control distribution.

As used herein, the terms “substantial difference”, “substantially different”, “substantially higher”, “substantially lower” refer to differences between values that are of a sufficient magnitude to enable reliable identification of a particular effect, e.g., the effect of administering a test agent to an animal on an electroencephalographic oscillation in the animal. Typically, a substantial difference is a difference that is statistically significant according to an appropriate statistical test, e.g., a Student's t-test, an ANOVA, etc. Substantial differences between two values may be about 1%, about 5%, about 10%, about 20%, about 30%, about 40%, about 50%, about 60%, about 70%, about 80%, about 90%, about 95%, about 99% depending on a variety of factors, including, for example, the nature of the value, the number of samples from which a value is derived, the statistical power of a comparison between two values, etc. Substantial differences may also be identified between two statistical distributions, e.g., by using an appropriate statistical test to compare distributions.

Similarly, as used herein, values that are “substantially equal” are values that can not reliably be established to be different. Typically, substantially equal values are values between which a statistically significant difference is not found, using an appropriate statistical test, e.g., a Student's t-test, an ANOVA, etc. Thus, substantially equal values may in fact be different, but the differences are not statistically significant. When values that represent a characteristic of an electroencephalographic oscillation are substantially equal the characteristic of the encephalographic oscillation is considered to be essentially the same. Also, statistical distributions (e.g., distributions of power of electroencephalographic oscillations) may be compared and determined to be substantially equal, e.g., by using an appropriate statistical test to compare distributions, e.g., Wilcoxon rank-sum test.

FIG. 17B illustrates an exemplary method of determining a distribution of power of EEG oscillation using spectral analysis, 107. At block 108, an EEG oscillation (e.g., an EEG oscillation, for example, a gamma, theta, or ripple oscillation, recorded at block 101) is obtained. The EEG oscillation will typically be in the form of a voltage time series of a particular duration (e.g., 30 minutes). In this embodiment, the EEG oscillation may be processed using two alternative approaches, which differ in the manner in which power of the EEG oscillation is determined (See block 111 and block 115). In either approach, at block 109_{1,2} the EEG oscillation is processed to obtain segments of EEG oscillation that have a predetermined duration.

At blocks 110₁₋₂, the power spectral density of the EEG oscillation segment is determined. Power spectral density (PSD) measures power per unit of frequency in an EEG oscillation. Any one of a variety of different methods may be used to determine the power spectral density of the EEG oscillation segment, including, for example, nonparametric and parametric methods. Nonparametric methods are those in which the PSD is estimated directly from the EEG oscillation segment itself. An example of such a method is the periodogram. Other nonparametric techniques include, but are not limited to, Welch's method and the multitaper method (MTM) both of which may reduce the variance of the periodogram. Parametric methods are those in which the PSD is estimated from a signal that is assumed to be output of a linear system driven by white noise. Non-limiting examples of parametric methods are the Yule-Walker autoregressive (AR) method and the Burg method.

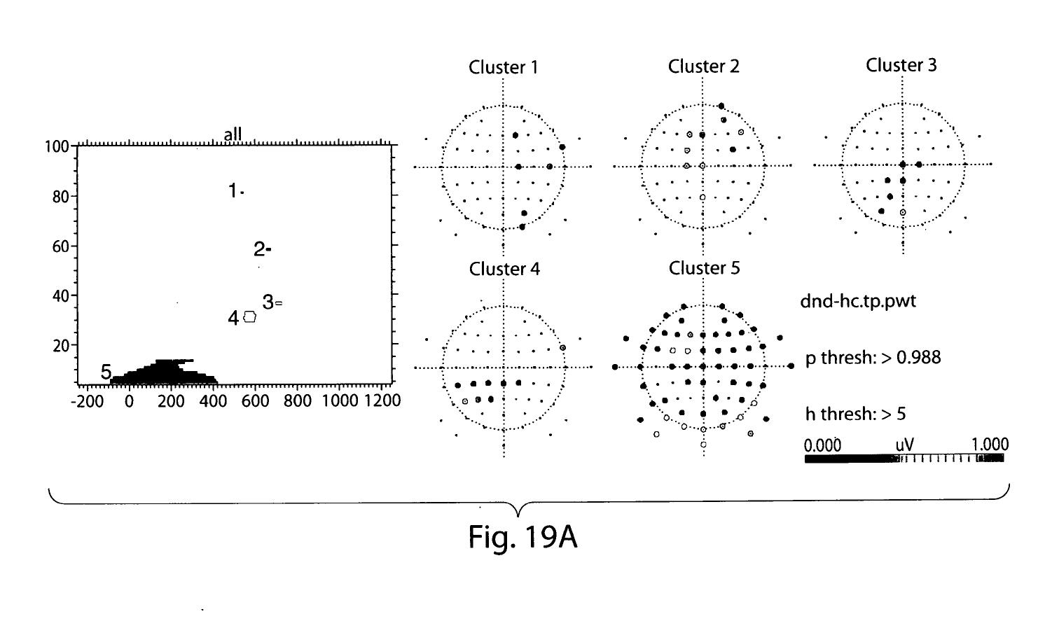

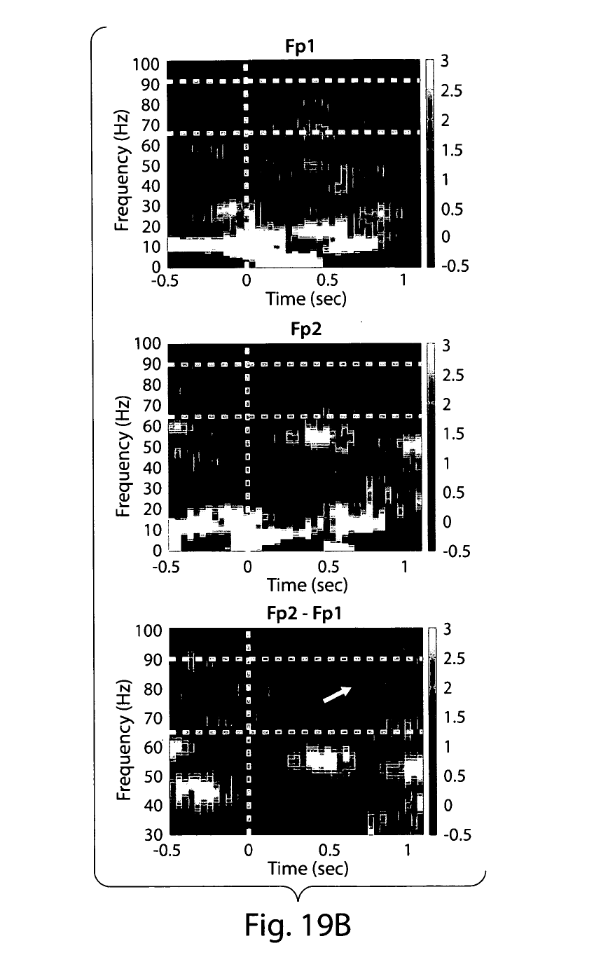

In some embodiments, the power spectral density of consecutive EEG oscillation segments may be plotted together (e.g., as a heat map.) Plotting power spectral density of consecutive EEG oscillation segments may be useful in some instances for detecting alterations in power (e.g., power peaks) that occur over a short period of time. For example, alterations may occur over short periods of time (e.g., 0.1 second to 1 second) in a subject who is engaged in a cognitive task that involves brief exposures to a novel object or visual stimulation (e.g., a visual novelty oddball task). In such cases, it may be desirable to produce and evaluate plots of power spectral density of consecutive EEG oscillation segments to detect alterations in power that occur over a short period of time (See, e.g., FIG. 19B).

In one alternative method of this embodiment, at block 111 power is determined from the EEG oscillation segment as the maximum value of the PSD within a predetermined frequency range. In another alternative method of this embodiment, at block 115 power is determined from the EEG oscillation segment as the area under the curve of the PSD function within a predetermined frequency range. The area under the curve of the PSD function may be obtained by integrating the PSD function (e.g., using trapezoidal numerical integration) across a predetermined frequency range. Power obtained using the area under the curve approach may be referred to herein as “ensemble EEG power”. The skilled artisan will appreciate that still other alternative methods for determining the power of the EEG oscillation segment may be used. For example, the arithmetic mean of the PSD function within a predetermined frequency range, the median of the PSD function with the predetermined frequency range, etc. In some embodiments, the power of the EEG oscillation segment is determined in the time domain. For example, the power may be estimated as the root mean square of an EEG oscillation segment that is a voltage time series, which may be a band-pass filtered voltage time series.

The predetermined frequency range of the PSD, from which the power of the EEG oscillation segment is determined, may be a frequency range corresponding to a gamma oscillation (e.g., 30 Hz to 90 Hz). In some embodiments, the predetermined frequency range corresponds to the upper portion of the gamma oscillation range (e.g., 65 Hz to 90 Hz). Other appropriate frequency ranges are disclosed herein and will be apparent to the skilled artisan.

At block 112_{1,2}, the power determined at 111 or at 115 is stored, e.g., in a database. The method branches at decision block 113_{1,2} where if additional EEG oscillations are to be obtained steps 109_{1,2} to 112_{1,2} are repeated. The method iterates through 109_{1,2} to 112_{1,2} until a sufficient number segments of EEG oscillations have been obtained to generate a distribution of power of EEG oscillations that is suitable for comparison with a control distribution for detection of a cognitive deficit. In some embodiments, the method iterates through 109_{1,2} to 112_{1,2} until essentially all of the recorded EEG oscillation is analyzed.

As provided above, at block 109_{1,2} the EEG oscillation segments have a predetermined duration. Typically, but not necessarily, that predetermined duration is the same for each segment obtained. It will also be appreciated that each EEG oscillation segment has the same time-dependent voltage content as the fraction of the EEG oscillation from which it was obtained. For example, an EEG oscillation segment may represent the first 10 second portion of the complete EEG oscillation. Another EEG oscillation segment may represent the next 10 second portion of the complete EEG oscillation, and so on. While typically the EEG oscillation segments are consecutive, in some instances, overlapping segments may be obtained.

When no additional EEG oscillation segments are to be obtained, the method proceeds to block 114 where a distribution (frequency histogram) of the power of the EEG oscillation is produced. The distribution is produced by binning the powers stored at block 112_{1,2}. As will be appreciated, depending on which alternative approach is used to determine the powers, the units of the binned power will vary. For example, if the maximum value approach is used, the power will typically be in units of voltage-squared versus frequency. Whereas, if the area under the curve approach is used, the power will typically be in units of voltage-squared.

### Screening Methods for Identifying Candidate Therapeutic Agents

According to some aspects of the invention, methods for identifying a candidate therapeutic agent for treatment of a cognitive deficit based on changes in electroencephalographic oscillations are provided. The methods may involve administering a test agent to a test animal, wherein the test animal has a neurological disorder or condition and/or is an animal model of a cognitive deficit and recording an electroencephalographic oscillation from the brain area of the test animal while the test animal is engaged in the cognitive task. The recorded electroencephalographic oscillation of the test animal is compared with a control electroencephalographic oscillation. Typically the comparison is made within a predetermined frequency range (e.g., within a gamma frequency range). A test agent that substantially reduces a difference between the electroencephalographic oscillation in the test animal compared to the control electroencephalographic oscillation, is identified as a candidate therapeutic agent for treatment of the cognitive deficit. Methods of the invention are appropriate for identifying candidate therapeutic agents that treat any of a variety of cognitive deficits. Typically, the cognitive deficit is characterized by an electroencephalographic oscillation, recorded from a brain area during a cognitive task, that substantially differs, in the predetermined frequency range, from a control electroencephalographic oscillation recorded from the brain area of a control animal during the cognitive task.

In some aspects of the invention, methods of the invention may be used to assess the ability of a test/candidate agent to alter gamma oscillations in an animal, such as a mouse, and agents that are found to alter the oscillations in the mouse may then be assessed in human clinical trials to determine whether the agent modulates a cognitive defect in a human subject. Thus, methods of the invention to assess test/candidate agents can be used as objective measures to assess candidate agents to treat a neurological disorder or condition, such as schizophrenia, etc. in clinical trials. In this way, methods of the invention provide biomarkers for neurological disorders or conditions and can be used in clinical trials to assess potential treatments for cognitive deficits. For example, power in a predetermined frequency range (e.g., gamma range (e.g., Gamma_{Hi} range)) of an electroencephalographic oscillation determined according to methods of the invention may serve as a biomarker. As another example, the distribution of the power of gamma oscillations determined according to methods of the invention may also serve as a biomarker. Thus, candidate agent assessment can be performed and any modulatory effect on a measured cognitive deficit can be validated using methods of the invention in human subjects having a cognitive deficit associated with a neurological disorder or condition.

The recorded electroencephalographic oscillation of the test animal may be compared with a control electroencephalographic oscillation by comparing power determined in the predetermined frequency range of the electroencephalographic oscillation of the test animal to power in the predetermined frequency range of the control electroencephalographic oscillation. For example, the recorded electroencephalographic oscillation of the test animal may be compared with a control electroencephalographic oscillation by comparing a frequency histogram of powers of the electroencephalographic oscillation to a frequency histogram of powers of the control electroencephalographic oscillation. The powers of the frequency histogram may be powers determined from predetermined time segments of the electroencephalographic oscillation. For example, the powers may be determined from the electroencephalographic oscillation by binning the signal into time segments (e.g., 10 sec segments) and each segment analyzed using power spectral analysis with Hamming windows (e.g., using Welch's method with Hamming windows). The recorded electroencephalographic oscillation of the test animal may be compared with a control electroencephalographic oscillation by comparing an average power of the electroencephalographic oscillation to an average power of the control electroencephalographic oscillation. As used herein, an “average power” of an electroencephalographic oscillation is a value that represents a typical power level in an electroencephalographic oscillation. For example, the average power may be the mean of power levels, the mode of power levels or the median of power levels in an electroencephalographic oscillation, or a component therefrom, e.g., a gamma oscillation component of an electroencephalographic oscillation.

In some embodiments, methods of the invention include administering a test agent to a test animal, where the test animal may have a neurological disorder or condition and/or may be an animal model of a cognitive deficit, and a cognitive deficit is characterized by a distribution of the power of electroencephalographic oscillations recorded from a brain area during a cognitive task that substantially differs from a control distribution of the power of electroencephalographic oscillations recorded from the brain area of a control animal during the cognitive task; recording electroencephalographic oscillations from the brain area of the test animal while the test animal is engaged in the cognitive task; determining the distribution of the power of electroencephalographic oscillations in the test animal during the cognitive task; and comparing the determined distribution of the power of electroencephalographic oscillations of the test animal to the control distribution of the power of electroencephalographic oscillations. Typically, a test agent that substantially reduces a difference between the distribution of the power of electroencephalographic oscillations in the test animal compared to the control distribution, is identified as a candidate therapeutic agent for treatment of the cognitive deficit. It is to be understood that the electroencephalographic oscillations in which differences between a test and control animal characterize a cognitive deficit may be of a variety of frequencies or frequency ranges.

Aspects of the method illustrated in FIG. 17B may be suitably implemented in a screen to identify candidate therapeutic agents for treating a cognitive deficit. For example, a test animal known to have a cognitive deficit may be administered a test agent in order to evaluate the ability of the test agent to affect the test animal's cognitive ability or performance in a manner desirable for a therapeutic agent that would treat the cognitive defect. At a time after administration of the test agent that is suitable for detecting an effect of the test agent on the cognitive ability or performance of the test animal, the test animal may be evaluated according to the method of detecting a cognitive deficit in a test animal, 100. If the comparison at block 103 of the method indicates a decrease in a difference between the distribution of power of EEG oscillation in the test animal and the control distribution, then the test agent may be identified as a candidate therapeutic agent for treating the cognitive deficit.

Aspects of the invention are based on the discovery that certain cognitive deficits are associated with novel signatures in electroencephalographic signals. For example, in normal animals in a familiar environment, primarily “Low” power gamma oscillations are observed in EEG signals obtained from the prefrontal cortex (PFC). Whereas, when normal animals are in a novel environment, “High” power gamma oscillations are observed in EEG signals obtained from the prefrontal cortex. Without wishing to be bound by theory, the results disclosed herein indicate that “High” power gamma oscillations may reflect higher cognitive function in the PFC. Therefore, disruption of cognition either through genetic or pharmacological or surgical means may shift basal gamma power up but may also inhibit expression of “High” power gamma oscillations. For example, data disclosed herein by way of the examples indicate that a cognitive deficit induced by PCP is not merely the result of an increase in gamma oscillation power, but rather may reflect a loss in discriminatory fidelity between active and resting states of PFC neural networks. Accordingly, a candidate therapeutic may be identified as an agent that shifts (e.g., reduces) the power of gamma oscillations to normal levels under a situation of baseline cognitive engagement, as represented by the familiar environment. Results disclosed herein by way of the examples differ from the results in published reports that describe neural activity and psychosis (Sebban, Tesolin-Decros et al. 2002; Pinault 2008; Ehrlichman, Gandal et al. 2009), in part, because the methods producing the results disclosed herein involve exposing animals to a novel environment, which engages the PFC and recruits attentional processes and executive function in the animals. Without wishing to be bound by theory the bimodal distribution of gamma oscillation power disclosed herein (e.g., in FIGS. 1 and 16) in normal control animals in a novel environment represents a mixture of resting and active states in PFC neural networks.

In some embodiments, induction of psychosis, either through genetic or pharmacological means, results in an increase in the power of gamma oscillation observed in EEG signals obtained from the PFC to an “Intermediate” power level. As used herein, an “Intermediate” power is a power having a level that is higher than the level of power of gamma oscillations observed when animals are in a familiar environment and is lower than the level of power of gamma oscillations observed when animals are in a novel environment. Gamma oscillations in the prefrontal cortex (PFC) in two distinct disease models are found to coalesce around an “Intermediate” power state, whereas gamma oscillations in the normal control animals are found to have a bimodal distribution of “High” and “Low” power states. It is shown that the “High” power state occurs during attending behavior and the “Low” power state occurs at baseline. The “Intermediate” power state does not permit sufficient discrimination of signal versus baseline, and provides a novel neurophysiological correlate of impaired higher cognitive function. This body of data, including the “Intermediate”, “Low” and “High” power gamma signatures provides the basis for novel assays to identify pro-cognitive agents. In some embodiments of the disclosure, an effective agent is an agent that shifts the “Intermediate” gamma power signature in the disease state toward or to a “High” power mode during instances of attending or higher level cognitive processes and/or toward or to a “Low” power mode during baseline behavior in which higher level cognitive processes are not recruited. Thus, an effective agent may be an agent that shifts power gamma signatures closer to, or to, the normal state, e.g. the state of gamma power signature in an animal that does not have impaired higher cognitive function. The present disclosure provides novel in vivo screening assays to identify modulators of gamma oscillation power, including, but not limited to, bimodal modulators of gamma oscillation power.

In some embodiments, the present disclosure provides methods including steps of administering candidate therapies to rodents and recording neural activity from the PFC of the rodents while they perform cognitive tasks including, but not limited to, novel object recognition, Delayed Non-Match-To-Position, 5 choice serial reaction time test, alternating T-maze, Set Shifting, 8-arm radial maze, odor spanning tasks, novelty oddball tasks. In some embodiments, the present disclosure provides in vivo screening methods to identify compounds for treatment of cognitive deficits in schizophrenia.

In some embodiments, inventive methods in accordance with the present disclosure can be used to identify agents for treatment of other disorders of cognition, such as, for example, bipolar disorder, Alzheimer's disease, Parkinson's disease, Huntington's Disease, Attention Deficit Hyperactivity Disorder (ADHD), multiple sclerosis, autism, or anxiety.

Methods are also provided for identifying therapies, by measuring effects of candidate agents on the power distribution of gamma oscillations in animal models of psychosis or cognitive impairment. Thus, an agent that modulates an abnormal gamma oscillation power distribution by making it more similar to a normal gamma oscillation power distribution may be useful as therapy for a cognitive deficit. In some aspects of the disclosure, agents that can restore gamma power so that it is more similar to, or the same as, the appropriate distribution of “High” and/or “Low” power during states of attention, executive function and higher cognitive processes, and baseline cognitive states, respectively, are identified as candidates for treating cognitive deficits in humans. Such agents are herein defined as “bimodal modulators of gamma oscillations” or as “modulators of gamma oscillations.” In some aspects of the disclosure, methods of identifying an agent as a modulator or bimodal modulator of gamma oscillation power distribution may include determining an effect of the agent on gamma oscillation power distribution in a test animal that is a model of a cognitive deficit, and comparing the determination with a determination of gamma oscillation power distribution in a control animal.

### Test Agents/Candidate Agents

As used herein, the term “test agent” or “candidate agent” are used interchangeably to refer to a compound or composition that is evaluated in a cellular, biochemical, or in vivo assay for its suitability as a candidate therapeutic agent. Without limitation, the following provides examples of test or candidate agents that made be used in the methods disclosed herein. Those of ordinary skill in the art will recognize that there are numerous additional types of suitable test agents that may be evaluated using the methods. Test agents can be small molecules (e.g., compounds that are members of a small molecule chemical library). The agents can be small organic or inorganic molecules of molecular weight below about 3,000 Daltons. The small molecules can be, e.g., from at least about 100 Da to about 3,000 Da (e.g., between about 100 to about 3,000 Da, about 100 to about 2,500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

Small molecules can be natural products, synthetic products, or members of a combinatorial chemistry library. A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art (e.g., as exemplified by Obrecht and Villalgrodo, Solid-Supported Combinatorial and Parallel Synthesis of Small-Molecular-Weight Compound Libraries, Pergamon-Elsevier Science Limited (1998)), and include those such as the “split and pool” or “parallel” synthesis techniques, solid-phase and solution-phase techniques, and encoding techniques (see, for example, Czarnik, A. W., Curr. Opin. Chem. Biol. (1997) 1:60). In addition, a number of small molecule libraries are publicly or commercially available (e.g., through Sigma-Aldrich, TimTec (Newark, Del.), Stanford School of Medicine High-Throughput Bioscience Center (HTBC), and ChemBridge Corporation (San Diego, Calif.).

In some embodiments, test agents are peptide or peptidomimetic molecules. In some embodiments, test agents include, but are not limited to, peptide analogs including peptides comprising non-naturally occurring amino acids, phosphorous analogs of amino acids, amino acids having non-peptide linkages, or other small organic molecules. In some embodiments, the test compounds are peptidomimetics (e.g., peptoid oligomers, e.g., peptoid amide or ester analogues, D-peptides, L-peptides, oligourea or oligocarbamate); peptides (e.g., tripeptides, tetrapeptides, pentapeptides, hexapeptides, heptapeptides, octapeptides, nonapeptides, decapeptides, or larger, e.g., 20-mers or more); cyclic peptides; other non-natural peptide-like structures; and inorganic molecules (e.g., heterocyclic ring molecules). Test agents can also be nucleic acids, including, e.g., shRNA, siRNA, microRNA, microRNA inhibitors (e.g., microRNA sponges), nucleic acid aptamers. In some embodiments, methods of the invention are used to evaluate, as test agents, “approved drugs”. An “approved drug” is any compound (which term includes biological molecules such as proteins and nucleic acids) which has been approved for use in humans by the FDA or a similar government agency in another country, for any purpose.

It will be understood that a therapeutic agent may reduce or eliminate a symptom of a disease, deficit, or disorder and may, but need not, eliminate the disease, deficit, or disorder. A therapeutic agent may delay onset of the disease, deficit, or disorder; shorten the duration of the disease, deficit, or disorder; eliminate the disease, deficit, or disorder in part; reduce the severity of one or more symptoms of the disease, deficit, or disorder; or eliminate the disease, deficit, or disorder entirely. Candidate therapeutic agents can be systematically altered, e.g., using rational design, to achieve (i) improved potency, (ii) decreased toxicity (improved therapeutic index); (iii) decreased side effects; (iv) modified onset of therapeutic action and/or duration of effect; and/or (v) modified pharmacokinetic parameters (absorption, distribution, metabolism and/or excretion).

The agents disclosed herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or as an aerosol. Thus, a variety of administration modes, or routes, are available. The particular mode selected will depend, of course, upon the particular test agent selected and the dosage required. Preferred modes of administration are parenteral and oral routes. The term “parenteral” includes subcutaneous, intravenous, intramuscular, intraperitoneal, and intrasternal injection, or infusion techniques. Other appropriate routes will be apparent to one of ordinary skill in the art.

According to the methods of the invention, the agents may be administered in a pharmaceutical composition. Administering the pharmaceutical composition of the present invention may be accomplished by any means known to the skilled artisan. In addition to the active agent, the pharmaceutical compositions of the present invention typically comprise a pharmaceutically-acceptable carrier. Pharmaceutically acceptable compositions can include diluents, fillers, salts, buffers, stabilizers, solubilizers and other materials which are well-known in the art. The term “pharmaceutically-acceptable carrier”, as used herein, means one or more compatible solid or liquid filler diluents or encapsulating substances which are suitable for administration to a human or lower animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being corn ingled with an agent, and with each other, in a manner such that there is no interaction which would substantially reduce the pharmaceutical efficacy of the pharmaceutical composition under ordinary use situations. Pharmaceutically-acceptable carriers must, of course, be of sufficiently high purity and sufficiently low toxicity to render them suitable for administration to the human or lower animal being treated.

Some examples of substances which can serve as pharmaceutically-acceptable carriers are sugars such as lactose, glucose and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; pyrogen-free water; isotonic saline; phosphate buffer solutions; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. The choice of pharmaceutically-acceptable carrier to be used in conjunction with the agents of the present invention is basically determined by the way the agent is to be administered. Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

The agents of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

The pharmaceutically acceptable carrier employed in conjunction with the agents of the present invention is used at a concentration sufficient to provide a practical size to dosage relationship. The pharmaceutically-acceptable carriers, in total, may comprise from about 60% to about 99.99999% by weight of the pharmaceutical compositions of the present invention, e.g., from about 80% to about 99.99%, e.g., from about 90% to about 99.95%, from about 95% to about 99.9%, or from about 98% to about 99%.

### Methods for Diagnosing and Monitoring Cognitive Deficits

In certain aspects of the invention methods of detecting a cognitive deficit in an animal are provided. In some cases, the methods are useful for diagnosing a cognitive deficit in an animal, e.g., for diagnosing a human or a non-human primate, as having a cognitive deficit. In other cases, the methods are useful for monitoring a cognitive deficit in an animal. For example, an animal, e.g., a human, having or suspected of having a cognitive deficit or a disease associated with a cognitive deficit may be monitored to evaluate the animal's response to a particular treatment. Treatment for the cognitive deficit may involve, for example, administering a procognitive therapeutic agent, an antipsychotic, an antiepileptic, an antidepressant, an anti-dementia, or an anti-anxiety medication or agent to the animal, depending on the type of cognitive deficit or disease. Treatment may also involve, for example, psychiatric or psychological counseling. Typically, the animal being monitored or diagnosed has or is suspected of having a cognitive deficit that is characterized by an electroencephalographic oscillation, recorded from a brain area during a cognitive task, that substantially differs, in a predetermined frequency range, from a control electroencephalographic oscillation recorded from the brain area of a control animal during the cognitive task. The methods may involve, for example, (a) recording an electroencephalographic oscillation from the brain area of the animal while the animal is engaged in the cognitive task; (b) comparing, in the predetermined frequency range, the electroencephalographic oscillation recorded in (a) of the animal to a control electroencephalographic oscillation, wherein a substantial difference between the electroencephalographic oscillation in the animal compared to the control electroencephalographic oscillation, indicates that the animal has a cognitive deficit. In some embodiments, steps (a) and (b) are repeated one or more times, thereby monitoring the cognitive deficit status in the animal over time. The animal may be administered a treatment for the cognitive deficit or for a disease associated with the cognitive deficit within at least one time interval during the monitoring period. This facilitates monitoring of the response to the treatment over time.

In certain aspects of the invention methods of determining the efficacy of a treatment for a cognitive deficit in an animal, e.g., in a human, are provided. Again, the cognitive deficit is typically characterized by an electroencephalographic oscillation, recorded from a brain area during a cognitive task, that substantially differs, in a predetermined frequency range, from a control electroencephalographic oscillation recorded from the brain area of a control animal during the cognitive task. In some embodiments, the methods comprise: (a) recording an electroencephalographic oscillation from the brain area of the animal while the animal is engaged in the cognitive task; (b) determining a distribution of the power of gamma oscillations in the electroencephalographic oscillation recorded in (a) of the animal; (c) comparing, in the predetermined frequency range, the distribution of the power of gamma oscillations determined in (b) to a control distribution of the power of gamma oscillations, wherein a substantial difference between the distribution of the power of gamma oscillations in the animal compared to the control distribution, indicates that the animal has a cognitive deficit; (d) administering a treatment for the cognitive deficit to the animal; and (e) repeating steps (a) to (c) one or more times after administering the treatment in step (d), wherein a substantial decrease in a difference between the distribution of the power of gamma oscillations in the animal compared to the control distribution, indicates that the treatment is effective for treating the cognitive deficit.

A control electroencephalographic oscillation may be an electroencephalographic oscillation obtained from the animal at a different point in time, e.g., prior to treatment, prior to the onset of one or more symptoms of a disease associated with the cognitive deficit, etc. The control electroencephalographic oscillation may be an electroencephalographic oscillation obtained from a different animal, e.g., a normal animal, e.g., an animal who does not exhibit symptoms of a disease associated with the cognitive deficit, etc. The control electroencephalographic oscillation may be an electroencephalographic oscillation representative of a normal animal, e.g., a reference standard of an electroencephalographic oscillation.

### Treatment Methods

In other aspects, methods for treating cognitive deficits are provided that involve restoring the distribution of gamma power to a normal pattern corresponding to the state of cognitive engagement. In some aspects of the invention, restoration of the gamma power may be restoration to a pattern that is more similar to a normal pattern than that of the untreated pattern, but need not be full restoration to a normal pattern. In some embodiments, the present disclosure provides methods for treatment of individuals suffering from schizophrenia. In some embodiments, the present disclosure provides methods for treatment of individuals suffering from cognitive deficits associated with schizophrenia. In some embodiments, inventive methods in accordance with the present disclosure can be used for treatment of other disorders of cognition, such as, for example, bipolar disorder, Alzheimer's disease, Parkinson's disease, Huntington's Disease, Attention Deficit Hyperactivity Disorder (ADHD), multiple sclerosis, autism, or anxiety.

Methods of treatment for restoration of normal cognitive function may include therapeutic agents that induce or modulate the “Intermediate” power gamma oscillations into a distribution of both “Low” power gamma oscillations observed during resting periods, and “High” power gamma oscillations observed during periods of attending to environmental stimuli.

### Systems and Components for Implementing Aspects of the Methods for Detecting Cognitive Deficits

Aspects of the methods illustrated in FIG. 17 and disclosed elsewhere herein may be implemented in any of numerous ways. For example, the various methods or processes outlined herein may be coded as software that is executable on one or more processors that employ any one of a variety of operating systems or platforms. Such software may be written using any of a number of suitable programming languages and/or programming or scripting tools, and also may be compiled as executable machine language code or intermediate code that is executed on a framework or virtual machine. The MATLAB signaling processing toolbox (The MathWorks, Inc., Natick, Mass.) is an exemplary, but non-limiting, system that may be used for implementing certain aspects of the methods disclosed herein.

In this respect, aspects of the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed herein. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs, which when executed perform certain methods disclosed herein, need not reside on a single computer or processor, but may be distributed in a modular fashion among or between a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element or a list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Exemplary embodiments of the disclosure will be described in more detail by the following examples. These embodiments are exemplary of the disclosure, which one skilled in art will recognize is not limited to the exemplary embodiments.

### EXAMPLES

### Example 1

### Intermediate Gamma Power in CN Heterozygous KO Mice

Microwire bundle electrodes were stereotaxically implanted into the PFC of female C57Bl/6 animals and calcineurin knockout (CNKO) mice. The implantation coordinates used were: from Bregma: +0.37 cm rostral, +0.07 cm lateral, and −0.05 cm deep from brain surface. After a 7-day recovery period, EEG traces from PFC were recorded from freely behaving mice in a novel environment. One type of chamber used for the recordings was an operant chamber from (Coulbourn Instruments, Whitehall, Pa.). Recordings were bandpass filtered (30-90 Hz) to quantify gamma oscillations. Data were binned into 10 sec segments and analyzed using power spectral analysis with Hamming windows. Graph indicates frequency histogram (dots) and Gaussian fit (solid curves) of maximal gamma power during a 30 min recording session of 3 mice from each genotype.

Results: Continuous EEG recordings made during a 30 min session in a novel environment reveal striking differences in gamma oscillations in the PFC of CN KO mice. Normal control mice exhibit a bimodal distribution of gamma oscillation power, with Low power events centered around 2 μV²/Hz (“Low”) and a higher power peak at 5 μV²/Hz (“High”). Homozygous CN KO mice exhibited a unimodal distribution centered around the “Low” powered peak. Heterozygous CN KO mice exhibited a unimodal distribution centered at 2.8 μV²/Hz, which resides between the “Low” and “High” peaks (“Intermediate”). (FIG. 1)

### Example 2

### Intermediate Gamma Power in PCP-Induced Mouse Schizophrenia Model

Normal control C57Bl/6 mice were injected with Saline and PCP (5 mg/kg) sequentially while recordings were made from PFC. Injections began after a 17 min baseline period. Data were collected and analyzed as in Example 1.

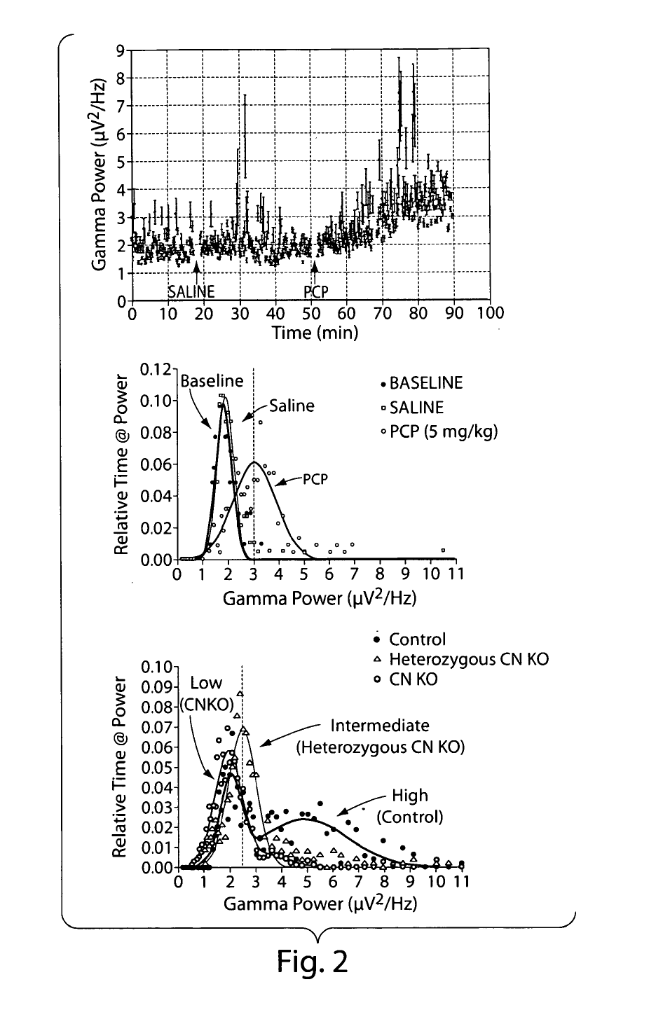

Results: Continuous EEG recordings were made in well-habituated animals. After a 17 min baseline period, animals were injected with saline. Thirty min after saline injection, animals received an injection of PCP (5 mg/kg). Injection of saline had no effect on gamma oscillation power. Injection of PCP induced an increase in gamma oscillation power to 4 μV²/Hz. This increase in gamma oscillation power is similar to the basal gamma oscillation power observed in heterozygous CN KO animals. Increases in gamma oscillation power in response to NMDA-R antagonists, such as PCP and MK-801, have been observed in various brain regions of rodents (Sebban, Tesolin-Decros et al. 2002; Pinault 2008; Ehrlichman, Gandal et al. 2009) (FIG. 2).

### Example 3

### Schematic of Therapeutic Signature

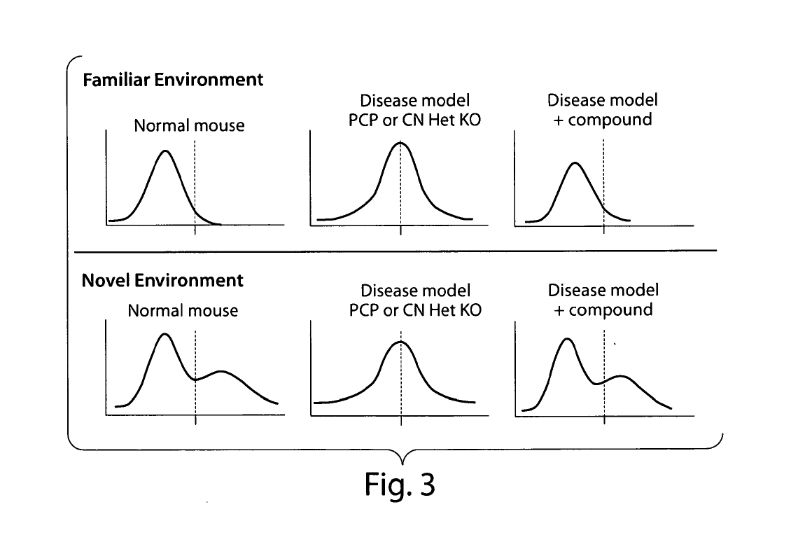

In the normal, healthy state, under baseline conditions such as familiar environment, gamma power in PFC is distributed in a unimodal “Low” state while under conditions inducing attention or higher cognitive engagement, gamma power in PFC is distributed in a bimodal distribution of “High” and “Low” power states, representing a mixture of cognitive baseline and engagement (FIG. 3, left hand panel). In the disease state, gamma is distributed at “Intermediate” power range in PFC, resulting in an inability to discriminate signal from background (FIG. 3, middle panel). Thus, some effective therapies will restore gamma power in PFC to “Low” power distribution during baseline states, and/or to restore a bimodal distribution of “High” and “Low” power during states in which attentional or higher level cognitive processes are active (FIG. 3, right panel).

### REFERENCES FOR REFERENCES 1 TO 3

Ehrlichman, R. S., M. J. Gandal, et al. (2009). “N-methyl-d-aspartic acid receptor antagonist-induced frequency oscillations in mice recreate pattern of electrophysiological deficits in schizophrenia.” Neuroscience 158(2): 705-12.
Pinault, D. (2008). “N-methyl d-aspartate receptor antagonists ketamine and MK-801 induce wake-related aberrant gamma oscillations in the rat neocortex.” Biol Psychiatry 63(8): 730-5.
Sebban, C., B. Tesolin-Decros, et al. (2002). “Effects of phencyclidine (PCP) and MK 801 on the EEGq in the prefrontal cortex of conscious rats; antagonism by clozapine, and antagonists of AMPA-, alpha(1)- and 5-HT(2A)-receptors.” Br J Pharmacol 135(1): 65-78.

### Example 4

### In Vivo Methods for Preclinical Analysis of Cognitive Therapies

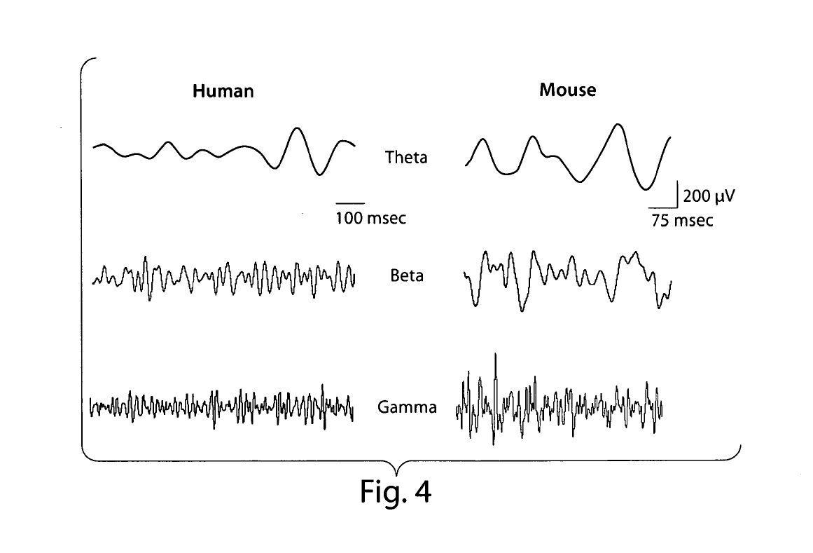

The prefrontal cortex (PFC) is important for executive function in both rodents and humans [14-16]. Synchronous neural activity in the gamma frequency band, a type of high frequency neural activity that is associated with higher cognitive function, occurs in the PFC of both humans and mice [3] (FIG. 4). Moreover, gamma oscillations in the PFC of patients with schizophrenia are significantly disrupted, especially during performance of higher level cognitive tasks requiring attention and working memory [3]. Based in part on this degree of innate functional conservation, cognitive assays have now been developed in animal models that are useful for effectively predicting the therapeutic potential of novel cognitive agents in the clinic. In vivo screening platforms are disclosed herein that fuse rodent behavioral analysis with real-time monitoring of neural activity in brain regions relevant for the cognitive domains that are altered in schizophrenia. By combining conventional and relevant behavioral tasks with real-time monitoring of neural activity in the PFC, a new cognitive assay platform has been established that is both sensitive to and specific for the deficits in executive function that are a hallmark of the cognitive deficits that occur in schizophrenia patients.

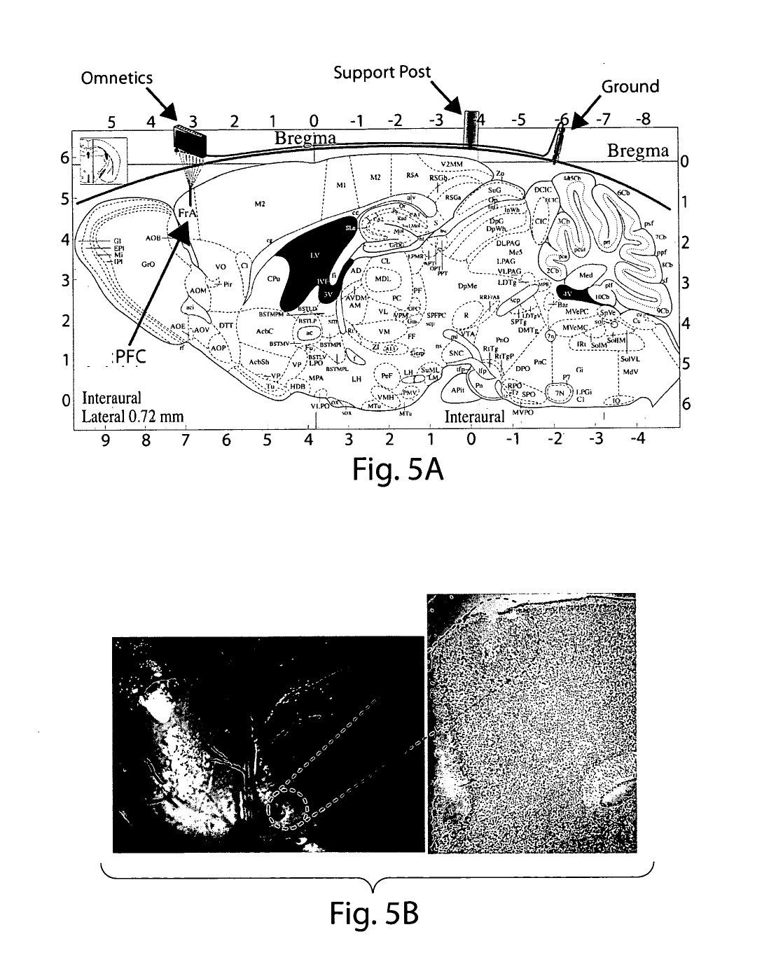

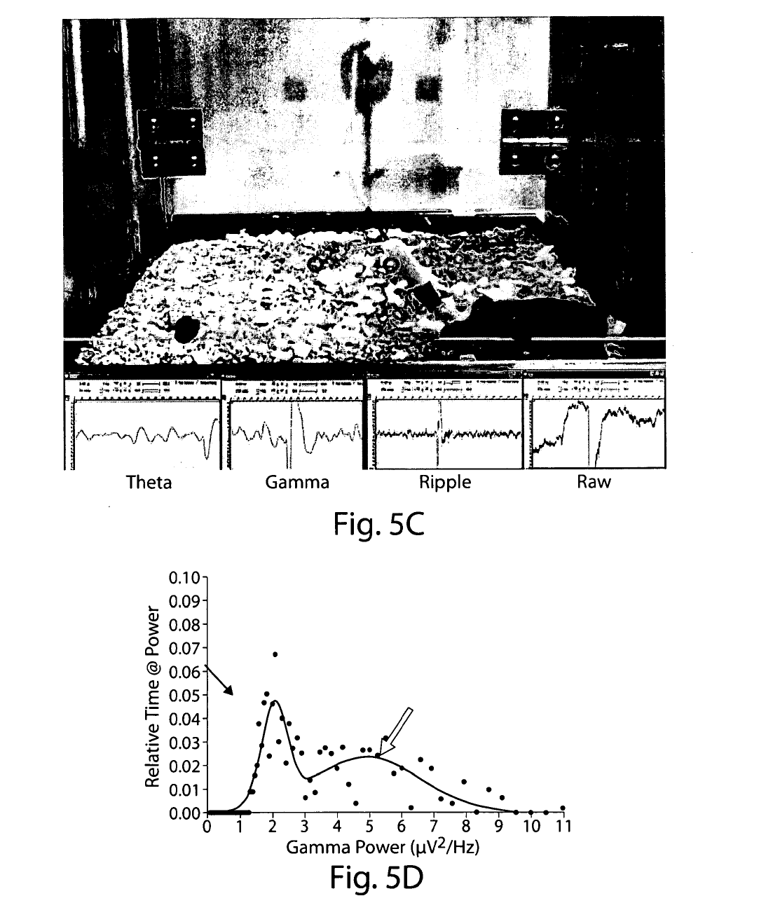

Real-time monitoring of EEGs and single-unit activity (SUA) from PFC was performed via surgical implantation of 8-microwire bundle electrodes (FIG. 5A, B). Implantation coordinates used were: from Bregma: +0.37 cm rostral, +0.07 cm lateral, and −0.05 cm deep from brain surface. Recordings were made using commercially available multichannel hardware (Multichannel Systems, Reutlingen, Germany). The system is capable of monitoring EEGs and SUA from PFC in freely behaving mice (FIG. 5C) and this neural activity has been characterized (FIG. 5D).

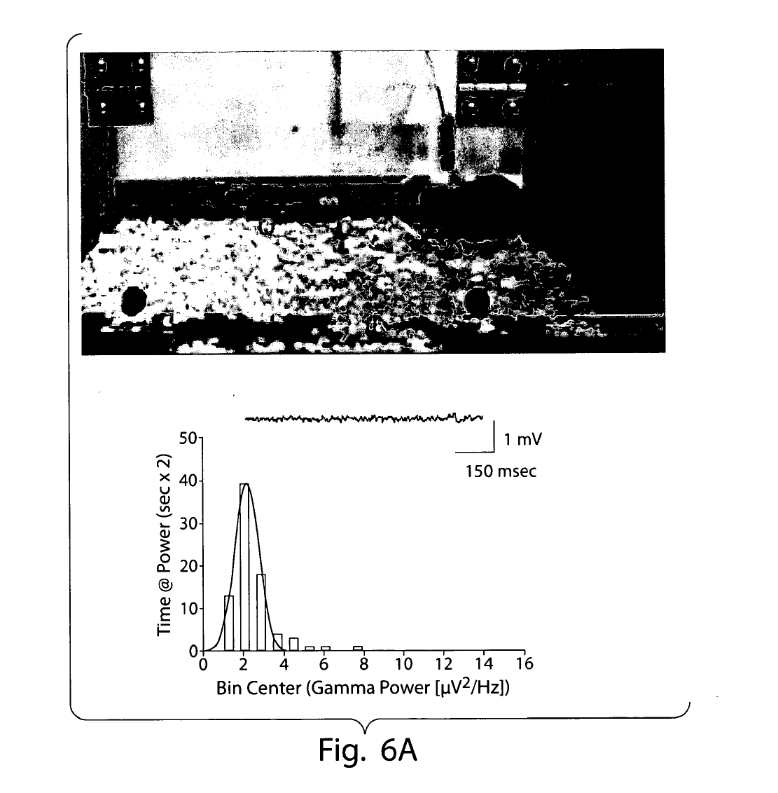

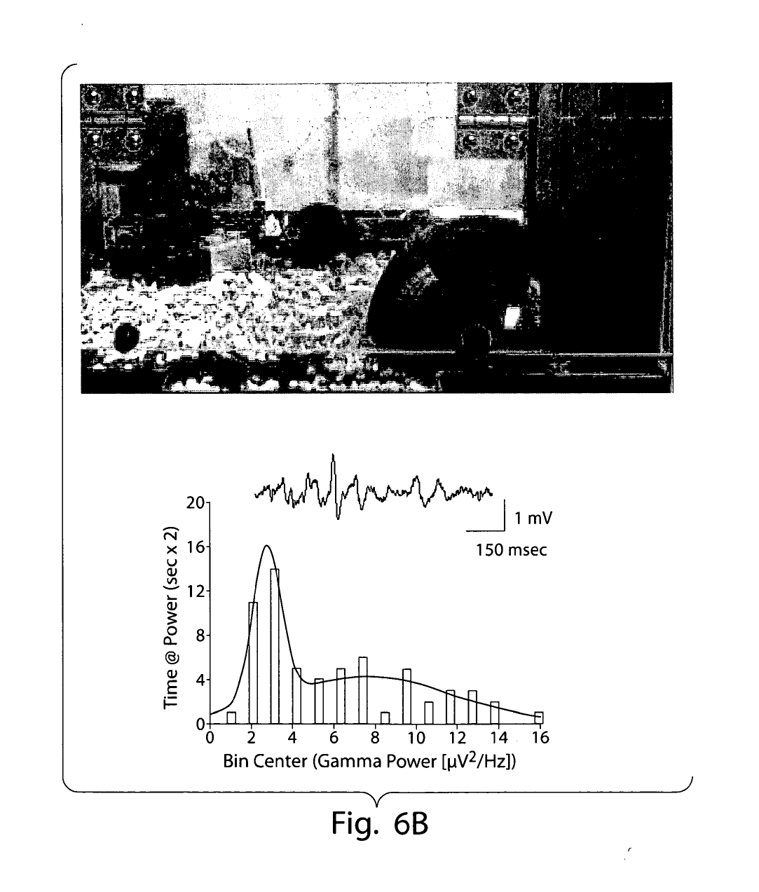

As part of the characterization of spontaneous EEGs and SUA recorded from PFC in freely behaving mice, a bimodal distribution of gamma oscillations as a function power was discovered (FIG. 5D). Specifically, oscillations appeared to coalesce around a lower and a higher power state (FIG. 5D). This bimodal distribution was observed when animals were first introduced into a novel environment. As animals were habituated across days to this environment, the high power oscillations disappeared. This suggested that these high power oscillations were related to attentional processes. To substantiate this, novel objects were presented to well-habituated animals, which resulted in a shift from the low power state, which was present in the absence of environmental stimuli (FIG. 6A), to a bimodal distribution consisting of both low and high power gamma oscillations in the presence of novel environmental stimuli (FIG. 6B). Interestingly, shifts in the power of cortical gamma oscillations have been observed in primates while performing attentional tasks [3]. The results demonstrate that the assay is capable of detecting changes in EEG power in the PFC of freely behaving animals in response to behaviorally salient stimuli.

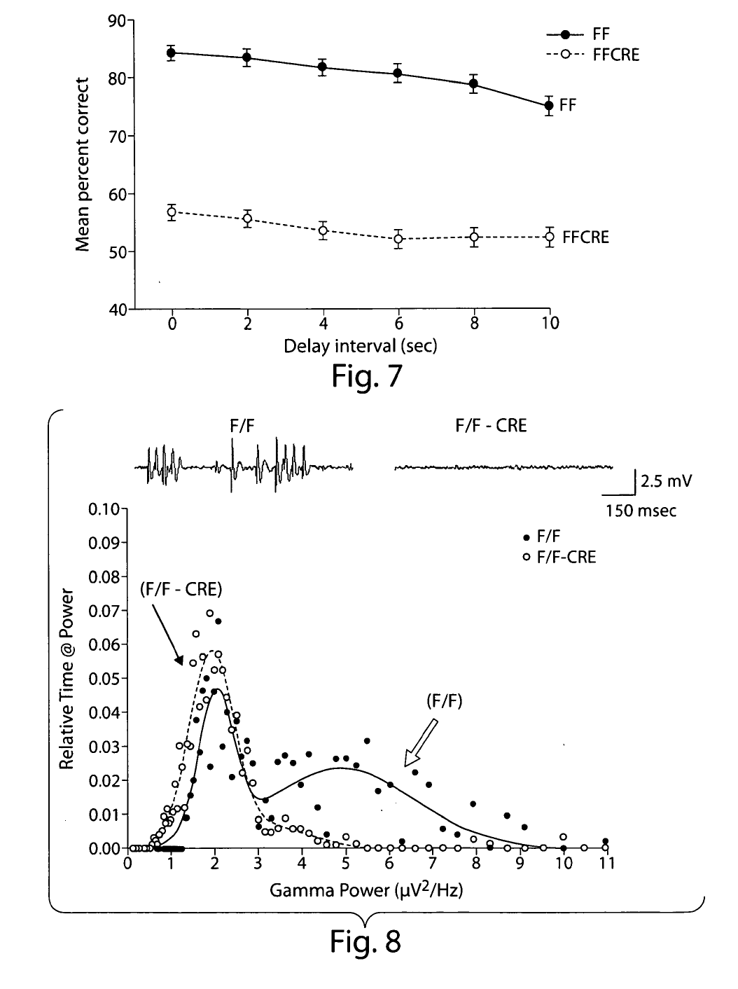

Gamma oscillations specifically and neural activity in general in the PFC have been associated with executive function and performance in working memory tasks [3]. Therefore, to determine whether the recording techniques are sensitive enough to detect changes in EEGs that are associated with performance of cognitive tasks that engage PFC in rodents, EEGs and SUA were measured from mice where the protein phosphatase calcineurin was knocked out (CNKO mice) postnatally in forebrain neurons [17]. Previously published reports of these mice indicated that performance in most cognitive paradigms was normal, however these animals exhibited a profound impairment in the 8-arm radial maze working memory task [17, 18]. Moreover, studies have indicated a genetic association of calcineurin with schizophrenia [19]. The current studies determined that significant deficits exist in the ability of CNKO mice to perform the Delayed Non-Match-To-Position (DNMTP) working memory task (FIG. 7). Direct recording of EEGs from the PFC of CNKO mice revealed a significant alteration in gamma oscillations relative to littermate control mice (FIG. 8). Specifically, expression of high-power gamma oscillations in the PFC in response to exposure to a novel environment was completely absent in CNKO mice (FIG. 8), suggesting severe impairment in PFC function in these mice. Collectively, the results of the current studies demonstrate a severe disruption in the expression of gamma oscillations in the PFC of a genetic mouse model harboring a specific working memory deficit. These observations confirmed that the technique for recording EEGs and SUA from PFC in freely behaving mice has sufficient sensitivity to detect neural alterations that would predict cognitive status.

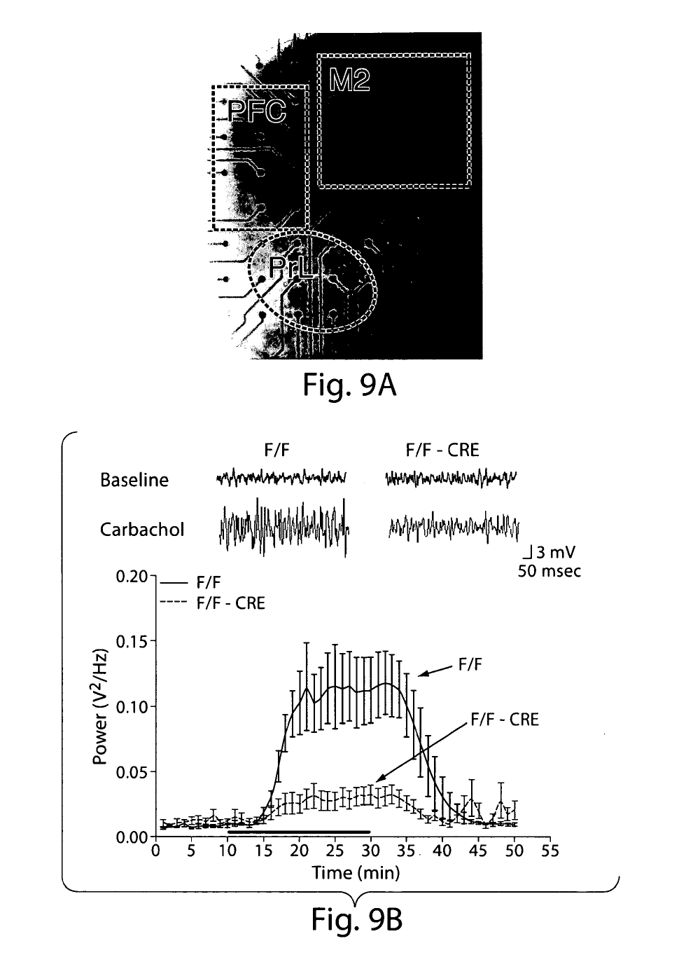

Without wishing to be bound to a particular theory or model, the deficits observed in neural activity in the PFC of CNKO animals in response to novelty could be due to a defect in neural function directly in the PFC; alternatively, derangement in PFC function could be secondary to a defect in another region of the brain that modulates PFC, such as hippocampus and/or the ventral tegmental area (VTA). To determine whether endogenous neural activity in the PFC is directly impaired in CNKO animals, EEGs and SUA from PFC were monitored in an acute brain slice preparation lacking afferents from hippocampus and VTA (FIG. 9A). Gamma oscillations were evoked by perfusing carbachol (20 μM) into the slice chamber [4]. We observed marked deficits in the ability to evoke gamma oscillations in the PFC of slices from CNKO mice relative to littermate controls (FIG. 9B). These data indicate that our observations in vivo are due, at least in part, to defects in neural function within PFC.

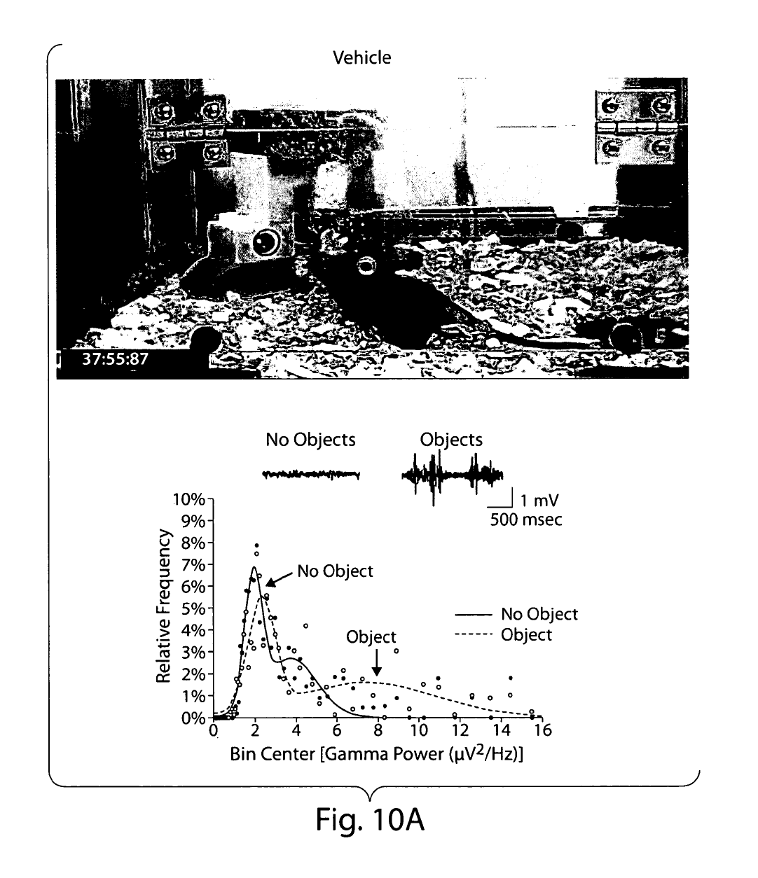

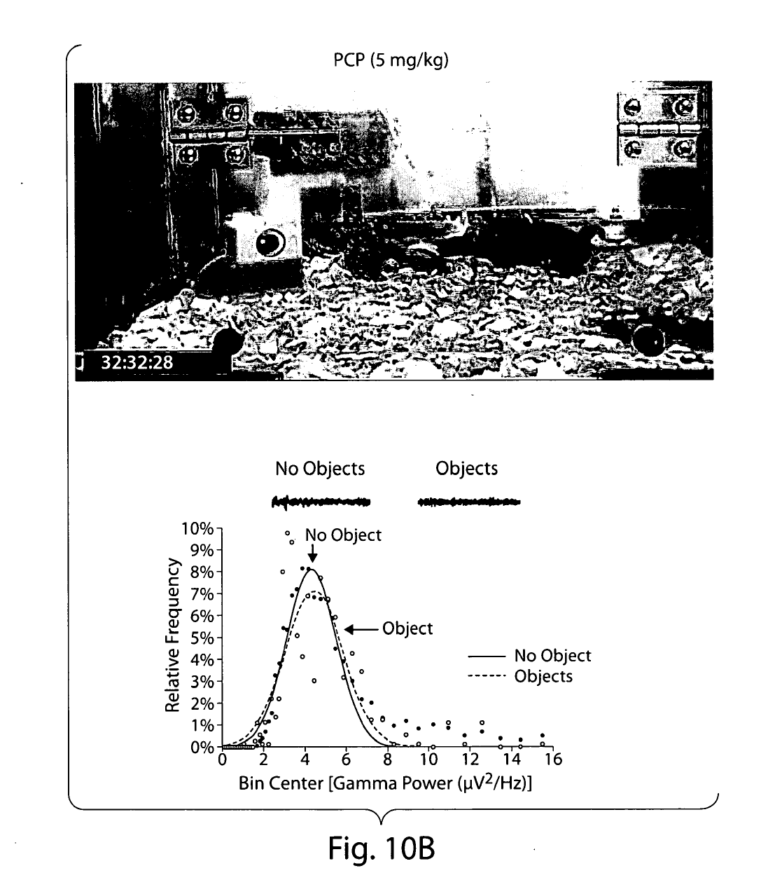

In another series of experiments, the EEG recording techniques were determined to be sensitive enough to detect pharmacologically-induced perturbations in neural activity in vivo. For these studies EEGs from PFC were measured when mice were in the absence and presence of novel environmental stimuli. To determine whether EEGs recorded from PFC are sensitive to a pharmacologic manipulation known to impair cognitive function, animals were injected intraperitoneally with PCP (5 mg/kg) and EEGs were recorded in the absence and presence of novel environmental stimuli. This experiment confirmed that high-power gamma oscillations in the PFC were elicited while animals attend to novel stimuli in the environment (FIG. 10A). Moreover, it was discovered that administration of PCP completely blocked the expression of high-power gamma oscillations in the PFC (FIG. 10B). These observations confirm that the techniques for monitoring EEGs and SUA in the PFC of freely behaving mice are sensitive enough to detect treatments known to inhibit cognition. Moreover, the results confirm the observed electrophysiological signature of higher cognitive function in the PFC through both genetic and pharmacologic manipulations.

### Example 5

### In Vivo Drug Screening Platform that Associates Neural Physiology Captured in Real-Time with Behavioral and Cognitive Outcomes

An integrated, “high-throughput” in vivo screening platform for identifying agents that specifically enhance aspects of cognition that are vital for executive function, attentional processes and working memory has been developed. The platform evaluates executive function, attention and working memory using novel object recognition. In vivo monitoring of EEGs from PFC was carried out during the novel object recognition behavioral paradigm to measure electrophysiological indices predictive of cognitive outcome in real-time. Power spectra were measured from 4 to 500 Hz to detect any change in synchronous neural activity within the PFC.

The integrated, “high-throughput” in vivo screening platform may also be developed to evaluate executive function, attention and working memory using DNMTP rodent behavioral paradigms. Recording techniques to detect SUA may be incorporated into the platform as well.

To enhance the sensitivity and throughput an automated behavioral analysis system was incorporated into the platform. Automated behavioral analysis permits real-time coding of electrophysiological data to specific behaviors, such as attending, stereotypy, overall movement, sniffing and other related data. For example, an MEA-60 (Multichannel Systems, Reutlingen, Germany) for multichannel amplification of electrophysiological signals and the TopScan automated behavioral monitoring system (Clever Sys, Inc., Reston, Va.) with operant chambers (Coulbourn Instruments, Whitehall, Pa.) are used. In some cases, measures of spontaneous activity that are quantified by automated tracking software are also mapped onto underlying neural activity in the PFC.

### Example 6

### Assay Validation Techniques

For assay validation, neural activity in the PFC and associated rodent behaviors are simultaneously evaluated following treatment by reference compounds that are known to enhance or interfere with attention and executive function. Drugs were administered either during the training or testing phases of the behavioral paradigms. The effects of compounds (e.g., cognitive enhancing compounds) on genetic (CNKO) and pharmacologic (subchronic PCP) models of the cognitive impairment were evaluated.

An exemplary modulator of cognition and PFC function is acetylcholine, which is released by ascending cholinergic projections from the brainstem and basal forebrain [21]. Disruption of these projections or modulation of the receptors for acetylcholine (nicotinic or muscarinic) modulates PFC-sensitive cognitive processes such as attention and working memory [22, 23]. Acetylcholine also acts at other regions in the brain that are important for cognition and also relevant to psychosis, such as hippocampus, striatum, and midbrain dopaminergic areas, such as the ventral tegmentum. Accumulating data suggests that altered cholinergic function is involved in schizophrenia etiology [24]. Recent clinical trials for drugs targeting the alpha7 nicotinic receptor and muscarinic receptors have provided some indication that modulation of these receptor systems can yield modest cognitive benefit [25, 26].

Modulation of dopamine D2 receptors via antagonism or partial agonism provides a mechanistic basis for most of the currently available therapies for schizophrenia. While such antipsychotic drugs (typical and atypical) have proven effective at treating positive symptoms, they have exhibited little or no effect in preclinical or clinical tests of cognition, especially with regard to working memory [27]. Therefore, these agents may serve as negative controls in validation experiments.

To validate an assay platform, a cholinergic agent that either interferes with or enhances cognitive function is used. To measure effects that occur during inhibition of cognitive function, scopolamine is used. Scopolamine is a non-specific muscarinic receptor antagonist that impairs both attention and working memory in animals and humans [22, 28, 29]. To measure effects that occur with enhanced cognitive function, agonists of the alpha7 nicotinic receptor are used. Exemplary agonists of the alpha7 nicotinic receptor include the commercially available compound, PHA543613.

### REFERENCES FOR EXAMPLE 4-6

1. Conn, P. J., et al., Schizophrenia: moving beyond monoamine antagonists. Molecular interventions, 2008. 8(2): p. 99-107.
2. Karakas, S., et al., Gamma response of the brain: a multifunctional oscillation that represents bottom-up with top-down processing. International journal of psychophysiology, 2001. 39(2-3): p. 137-50.
3. Uhlhaas, P. J., et al., The role of oscillations and synchrony in cortical networks and their putative relevance for the pathophysiology of schizophrenia. Schizophrenia bulletin, 2008. 34(5): p. 927-43.
4. Banos, M., I. Vida, and P. Jonas, Synaptic mechanisms of synchronized gamma oscillations in inhibitory interneuron networks. Nat Rev Neurosci, 2007. 8(1): p. 45-56.
5. Lodge, D. J., M. M. Behrens, and A. A. Grace, A loss of parvalbumin-containing interneurons is associated with diminished oscillatory activity in an animal model of schizophrenia. J Neurosci, 2009. 29(8): p. 2344-54.
6. Tort, A. B., et al., Dynamic cross-frequency couplings of local field potential oscillations in rat striatum and hippocampus during performance of a T-maze task. Proc Natl Acad Sci USA, 2008. 105(51): p. 20517-22.
7. McEvoy, J. P., The costs of schizophrenia. J Clin Psychiatry, 2007. 68 Suppl 14: p. 4-7.
8. Marder, S. R., The NIMH-MATRICS project for developing cognition-enhancing agents for schizophrenia. Dialogues Clin Neurosci, 2006. 8(1): p. 109-13.
9. Carter, C. S. and D. M. Barch, Cognitive neuroscience-based approaches to measuring and improving treatment effects on cognition in schizophrenia: the CNTRICS initiative. Schizophr Bull, 2007. 33(5): p. 1131-7.
10. Young, J. W., et al., Using the MATRICS to guide development of a preclinical cognitive test battery for research in schizophrenia. Pharmacol Ther, 2009. 122(2): p. 150-202.
11. Markou, A., et al., Removing obstacles in neuroscience drug discovery: the future path for animal models. Neuropsychopharmacology, 2009. 34(1): p. 74-89.
12. Sarter, M., Preclinical research into cognition enhancers. Trends Pharmacol Sci, 2006. 27(11): p. 602-8.
13. Wu, E. Q., et al., The economic burden of schizophrenia in the United States in 2002. J Clin Psychiatry, 2005. 66(9): p. 1122-9.
14. Barch, D. M., The cognitive neuroscience of schizophrenia. Annual review of clinical psychology, 2005. 1: p. 321-53.
15. Dailey, J. W., R. N. Cardinal, and T. W. Robbins, Prefrontal executive and cognitive functions in rodents: neural and neurochemical substrates. Neurosci Biobehav Rev, 2004. 28(7): p. 771-84.
16. Tan, H. Y., J. H. Callicott, and D. R. Weinberger, Dysfunctional and compensatory prefrontal cortical systems, genes and the pathogenesis of schizophrenia. Cereb Cortex, 2007.
17 Suppl 1: p. i171-81.
17. Zeng, H., et al., Forebrain-specific calcineurin knockout selectively impairs bidirectional synaptic plasticity and working/episodic-like memory. Cell, 2001. 107(5): p. 617-29.
18. Miyakawa, T., et al., Conditional calcineurin knockout mice exhibit multiple abnormal behaviors related to schizophrenia. Proc Natl Acad Sci USA, 2003. 100(15): p. 8987-92.
19. Gerber, D. J., et al., Evidence for association of schizophrenia with genetic variation in the 8p21.3 gene, PPP3CC, encoding the calcineurin gamma subunit. Proc Natl Acad Sci USA, 2003. 100(15): p. 8993-8.
20. Stone, M., et al., Working and strategic memory deficits in schizophrenia. Neuropsychology, 1998. 12(2): p. 278-88.
21. Briand, L. A., et al., Modulators in concert for cognition: modulator interactions in the prefrontal cortex. Prog Neurobiol, 2007. 83(2): p. 69-91.
22. Ellis, J. R., et al., Muscarinic and nicotinic receptors synergistically modulate working memory and attention in humans. Int J Neuropsychopharmacol, 2006. 9(2): p. 175-89.
23. Everitt, B. J. and T. W. Robbins, Central cholinergic systems and cognition. Annu Rev Psychol, 1997. 48: p. 649-84.
24. Lieberman, J. A., J. A. Javitch, and H. Moore, Cholinergic agonists as novel treatments for schizophrenia: the promise of rational drug development for psychiatry. Am J Psychiatry, 2008. 165(8): p. 931-6.
25. Freedman, R., et al., Initial phase 2 trial of a nicotinic agonist in schizophrenia. Am J Psychiatry, 2008. 165(8): p. 1040-7.
26. Shekhar, A., et al., Selective muscarinic receptor agonist xanomeline as a novel treatment approach for schizophrenia. Am J Psychiatry, 2008. 165(8): p. 1033-9.
27. Hagan, J. J. and D. N. Jones, Predicting drug efficacy for cognitive deficits in schizophrenia. Schizophr Bull, 2005. 31(4): p. 830-53.
28. Chudasama, Y., et al., Cholinergic modulation of visual attention and working memory: dissociable effects of basal forebrain 192-IgG-saporin lesions and intraprefrontal infusions of scopolamine. Learn Mem, 2004. 11(1): p. 78-86.
29. Estape, N. and T. Steckler, Cholinergic blockade impairs performance in operant DNMTP in two inbred strains of mice. Pharmacol Biochem Behav, 2002. 72(1-2): p. 319-34.
30. Wiig, K. A., et al., The levo enantiomer of amphetamine increases memory consolidation and gene expression in the hippocampus without producing locomotor stimulation. Neurobiol Learn Mem, 2009.
31. Levenson, J. M., et al., Regulation of histone acetylation during memory formation in the hippocampus. J Biol Chem, 2004. 279(39): p. 40545-59.
32. Ayala, J. E., et al., mGluR5 Positive Allosteric Modulators Facilitate both Hippocampal LTP and LTD and Enhance Spatial Learning. Neuropsychopharmacology, 2009.

### Example 7

### Spectral Analysis of Electroencephalographic (EEG) Oscillations in the Rodent PFC

Using the methods disclosed herein, a bimodal signature was observed for the power of gamma activity in EEGs recorded from the PFC of freely behaving normal control mice. This signature was altered in homozygous calcineurin knockout mice as these animals displayed a unimodal distribution of low power. This signature was also altered in heterozygous calcineurin knockout mice as these animals displayed a unimodal distribution of intermediate power. The calcineurin deficient mice have deficits in working memory compared with the normal mice. Thus, the EEG signatures in normal and calcineurin deficient mice appear to be associated with underlying intact and impaired cognitive processes, respectively.

### Methods

Surgery, Animal handling and EEG recording were performed according to the methods disclosed herein (See, e.g., Examples 1-5). Recordings in a novel environment were made upon an animal's first exposure to the recording chamber. Animals were exposed to the recording chamber an additional 3 times (15 min each) to record EEGs from a familiar environment (fourth exposure).

EEG analysis. EEGs were analyzed using an automated FFT-periodogram function with Hamming windows in the Matlab analysis suite (pwelch function, The Mathworks, Inc., Natick MA). EEGs were analyzed in 10 sec intervals and power spectral analyses were computed for each 10 sec interval by averaging 1 sec power spectra with a 0.5 sec overlap across the entire 10 sec. Power across a given frequency band was determined by computing the area under the curve for various frequency bands (Theta: 4-12 Hz; Gamma_{Low}: 30-55 Hz; Gamma_{Hi}: 65-90 Hz; Ripple: 100-300 Hz). Measures of power for each animal were binned and a frequency histogram representing number of events (10 sec each) observed at each power was computed. Frequency histograms across a given genotype were averaged to derive the final EEG power histogram for each frequency band.

### Results:

EEGs were recorded from the PFC of mice upon first presentation to an experimental chamber (“Novel environment”) and after repeated exposure to the same chamber (“Familiar Environment”). Control animals exhibited a significant increase in the number of high-powered episodes in the Gamma_{Hi} frequency band relative to CNₕₑₜKO or CNKO animals when exposed to a novel environment (FIG. 1A, p<0.0001). In contrast, EEGs recorded from a familiar environment revealed that CNₕₑₜKO animals exhibited a significant increase in the number of high-powered episodes in the Gamma_{Hi} frequency band relative to CNKO or littermate control animals (FIG. 1B, p<0.0001). Further analysis revealed that Gamma_{Hi} events recorded from control animals in a novel environment were significantly higher in power compared to Gamma_{Hi} events recorded in a familiar environment (FIG. 11A,B; t=50, df=31, p<0.001). In contrast, no significant differences were observed in the power of Gamma_{Hi} events recorded from CNₕₑₜKO (FIG. 11A,B; t=0.3, df=39, p>0.05) or CNKO animals (t=0.8, df=5, p>0.05) in novel versus familiar environment. These results indicate that in normal animals, spectral power in the Gamma_{Hi} frequency band correlates with novelty detection and/or exploratory behaviors. Loss of CN function impairs environmentally-induced shifts in Gamma_{Hi} EEG power in the PFC. Moreover, CNₕₑₜKO animals exhibit an average Gamma_{Hi} power that is locked in an intermediate power between the high-powered Gamma_{Hi} events observed in control animals in a novel environment and the low-powered Gamma_{Hi} events observed in control animals in a familiar environment.

To investigate the disease relevance of this intermediate power phenotype, EEGs were recorded from normal control animals in a familiar environment before and after they were treated with the psychotomimetic compound, phencyclidine (PCP) (5 mg/kg, IP). PCP induced a significant increase in the number of high-powered events in the Gamma_{Hi}, frequency band observed in control animals in a familiar environment (FIG. 11C, p<0.0001). Notably, this increase in Gamma_{Hi} power was not different from the average Gamma_{Hi} power observed in CNₕₑₜKO animals in a familiar environment (FIG. 11B,C). Collectively, these results indicate that the intermediate Gamma_{Hi} power observed in CNₕₑₜKO animals reflects a psychosis disease-relevant signature and supports a therapeutic approach, involving decreasing Gamma_{Hi} power in a familiar environment and increasing Gamma_{Hi} power in a novel environment.

As shown in FIG. 16, when spectral power analysis in the Gamma wide band (30-90 Hz) was measured in animals in a novel environment, control animals exhibit a bimodal distribution of power, with a sharp peak in the low power range and a broad peak in the high power range, consistent with the observations disclosed using the method of Example 1. Both heterozygous and homozygous knockout mice exhibit overlapping, low power peaks.

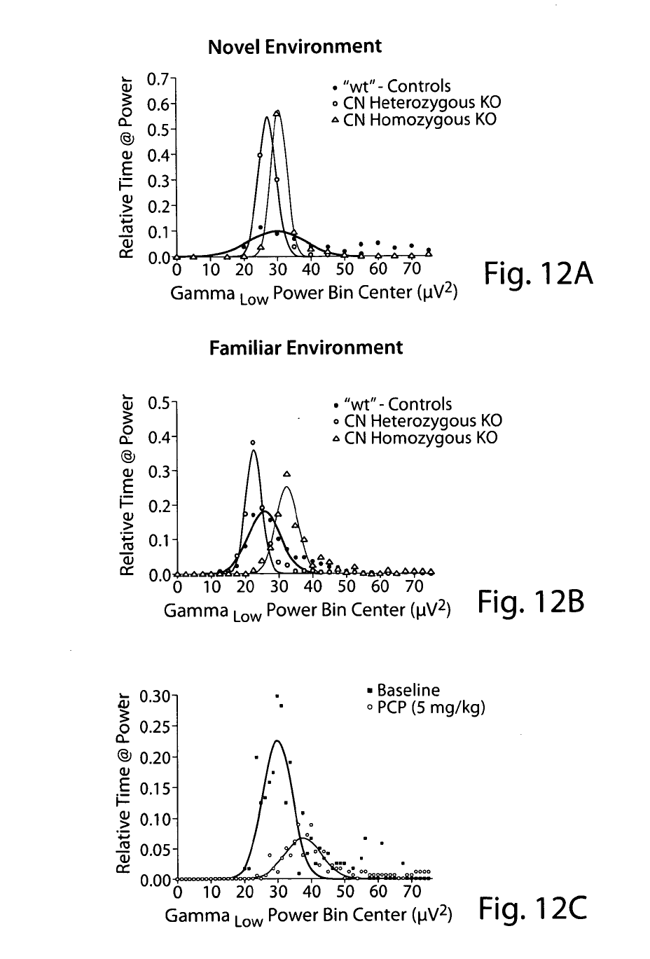

Additional frequency bands in the PFC EEG were analyzed to determine whether they are altered by genetic disruption of calcineurin or treatment with the psychotomimetic compound, PCP. Analysis of the Gamma_{Low} frequency band (30-55 Hz) indicates that CNKO animals consistently exhibit more high-powered events relative to CNₕₑₜKO animals in both novel (FIG. 12A, p<0.01) and familiar (FIG. 12B, p<0.001) environments. Moreover, treatment of control animals with PCP evokes significantly more high-powered Gamma_{Low} events (FIG. 12C, p<0.0001). Together, these results indicate that increases in Gamma_{Low} power in the PFC are associated with psychosis disease state.

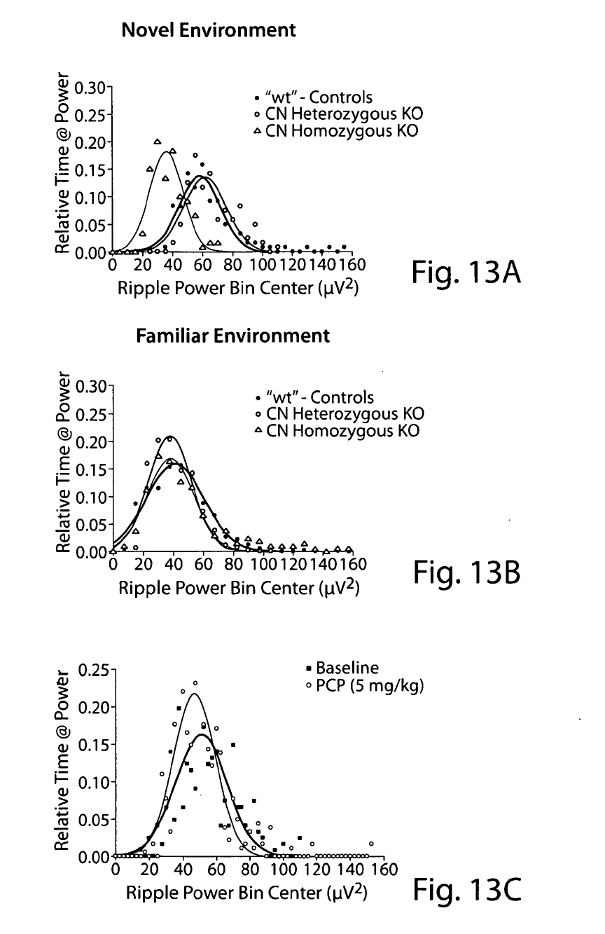

Examining the ripple frequency band (100-300 Hz), a significant diminution of spectral power was observed in CNKO animals in a novel environment (FIG. 13A, p<0.0001), but not in a familiar environment (FIG. 13B) relative to both CNₕₑₜKO and littermate control animals. Likewise, control animals treated with PCP exhibited no significant differences in power in the ripple frequency band in a familiar environment relative to untreated normal controls (FIG. 13C). These results suggest that decreases in ripple power are associated with severe cognitive impairment, but may not be associated with psychotic disease state.

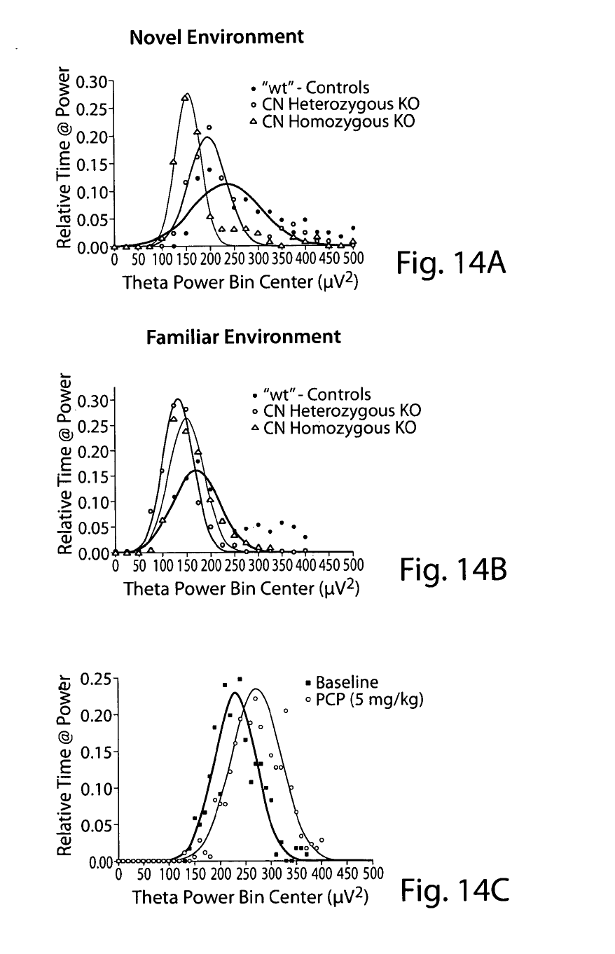

In an analysis of the theta frequency band (4-12 Hz), gene dosage-dependent decreases in average spectral power were observed in the theta frequency band recorded from CNKO and CNₕₑₜKO mice in a novel environment relative to normal controls (FIG. 14A, p<0.0001). Moreover, in control animals a significant increase was observed in theta power in a familiar environment after treatment with PCP (FIG. 14C, p<0.0001). These results suggest that relative power observed in the theta frequency band approximates relative power observed in the Gamma_{Hi} frequency band. This observation is consistent with other findings that theta oscillations may gate or modulate expression of gamma oscillations in various brain regions [1]. These findings indicate that power in the theta frequency band provides a biomarker for psychotic disease state, similar to oscillatory power in the Gamma_{Hi} frequency band. However, theta oscillations in the rodent PFC appear to be largely generated in the hippocampus, suggesting that this measure may not directly reflect the state of the neural circuitry in the PFC responsible for generating the high-frequency gamma oscillations necessary for executive function [1].

Several observations have been made regarding loss of calcineurin function and treatment with psychotomimetic substances and the state of the neural networks within the PFC, based on the spectral analysis of EEGs in the rodent PFC. Significant perturbations of gamma oscillations that provide reliable markers for psychotic disease state were observed to segregate to the Gamma_{Hi} frequency band. Changes in spectral power of the theta frequency band appear to correlate to changes in the Gamma_{Hi} band and may be useful as another marker for psychosis. Changes in the Gamma_{Low} and Ripple frequency bands may correlate with psychotic disease state. The data disclosed herein indicate that spectral power measured in discrete frequency bands from the PFC provides an objective measure for psychotic disease state.

### REFERENCE FOR EXAMPLE 7

1. Sirota, A., et al., Entrainment of neocortical neurons and gamma oscillations by the hippocampal theta rhythm. Neuron, 2008. 60(4): p. 683-97.

### Example 8

### Spectral Analysis of EEG Oscillations

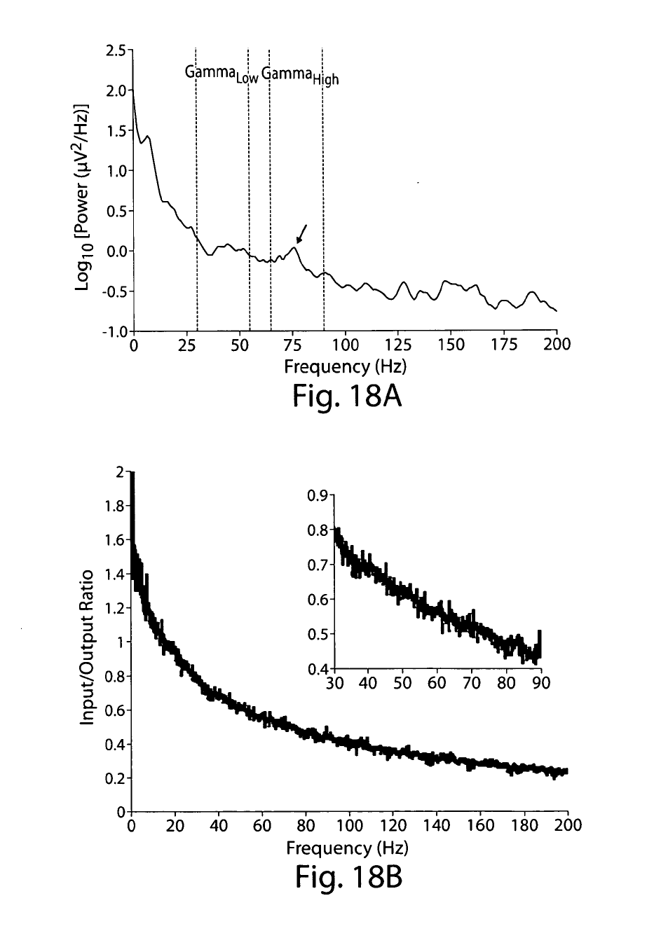

A power spectral density was determined for an EEG oscillation obtained from an animal (FIG. 18.) Two alternative approaches for spectral analysis of the recorded EEG oscillation were used to determine power at different frequency bands. In one approach, power was determined as the maximum power in the Gamma (30-90 Hz) band. In the other approach, power was determined using the area under the curve for frequency ranges corresponding to Gamma_{Low} and Gamma_{Hi} oscillations.

When power is determined as the maximum power in the Gamma band, events occurring in the Gamma_{Low} band may mask high power events in the Gamma_{Hi} band. In the example spectrum of FIG. 18A, a local maximum in power was observed at about 75 Hz, which is within the Gamma_{Hi} band. However, this local maximum is lower than the maximum power observed in the Gamma_{Low} band, and thus, is not the maximum power observed in the complete Gamma band. Factors contributing to these observations are the physical properties of the electrodes and normal decay in signal that occurs using power spectral analysis which may be referred to as the 1/Frequency effect (See FIG. 18B.) In some instances, it may be desirable to use an approach that minimizes the 1/Frequency effect. For example, when power is determined using the area under the curve, method, the signal:noise ratio may be maximized within certain frequency ranges, e.g., Gamma_{Hi} band, and the 1/Frequency effect may be minimized. In some embodiments, an EEG spectrum may be normalized to reduce or eliminate a 1/Frequency effect. In certain embodiments, power is determined from a normalized EEG spectrum. An EEG spectrum may be normalized by a control spectrum (e.g., a spectrum based on a chirp stimulus (e.g., as depicted in FIG. 18B)).

### Example 9

### Spectral Analysis of Electroencephalographic (EEG) Oscillations in the Human Frontal Cortex

### Introduction

To determine whether any aspects of neural oscillations in the human cortex were modulated in response to visual novelty presentation, Experiments were performed to investigate whether aspects of the EEG signatures measured in mice in response to environmental novelty were observed in humans. The results of the experiments indicated that aspects of neural oscillations in the human cortex were modulated in response to visual novelty presentation,

### Methods

### Subjects

Subjects were 10 healthy individuals (ages 38-52 years) recruited from the VA Schizophrenia Center's pool of control subjects. These individuals had participated in a number of EEG studies already. Subjects were selected without regard for ethnicity, and met the Schizophrenia Center's standard inclusion criteria: 1) age between 21-55 years; 2) right-handed (so that possible hemispheric lateralization effects would not be obscured by left-handers with reduced or reversed functional laterality); 3) no history of electroconvulsive treatment; 4) no history of neurological illness, including epilepsy; 5) no history of alcohol or drug dependence, nor abuse within the last year, nor long duration (>1 year) of past abuse (DSM-IV criteria); 6) no present medication for medical disorders that would have deleterious EEG, neurological, or cognitive functioning consequences; 7) verbal IQ above 75; 8) no alcohol use in the 24 hours prior to testing; and 9) English as a first language.

### Task

Subjects were seated in a comfortable chair in a darkened room and given a visual “novelty oddball” task. The stimuli were presented on a cathode ray tube computer monitor, situated 100 cm from the subject's nasion. Following Courchesne et al. (1975), there were 4 types of stimuli: targets (the letter “X”), standards (the letter “Y”), novels (complex, colored patterns), and “dims” (grey squares). Stimuli were approximately 3° X 3° of visual angle.

The task was divided into 6 blocks of 120 trials. Each block of trials consisted of 15 targets, 15 novels, 15 dims, and 75 standards. The interval between stimulus onsets was ˜1500 ms. Each stimulus was presented for 116 ms. The subjects' task was to press a button on the response box when a target stimulus was presented.

### EEG Recording and Processing

The EEG was continuously recorded at 512 Hz sampling rate using a 72-channel Biosemi ActiveTwo system at standard scalp electrode sites. Electrodes were also placed at just below the left eye and at the outer canthi of the left and right eyes for deriving the vertical and horizontal electro-oculograms (EOGs), respectively.

Following data acquisition, the EEG was segmented into epochs from −500 to 1498 ms relative to stimulus onset. To ensure that the standard condition had a similar signal-to-noise ratio as the other conditions, 15 standard trials were selected at random from each block for analysis instead of the full number of standards. The epochs were analyzed for artifacts using a criterion of +/−90 μV for amplitude, or greater than 150 μV amplitude range, on any channel. Independent component analysis was applied to remove EOG and other artifacts (muscle artifacts, bad channels). The artifact-free epochs were re-referenced to the average reference. A subject's data were excluded from further analysis if following artifact correction/rejection, a subject did not have at least 67 artifact-free trials in each condition summed across blocks (i.e., 75% artifact-free trials in each condition).

Event related potentials (ERP) were computed for each condition by averaging the single-trial epochs. Event-related time-frequency measures (total power and phase locking factor) were computed using the Morlet wavelet transform. The range of frequencies analyzed were 4-100 Hz (1 Hz resolution).

### Statistical Analysis

A statistical non-parametric mapping procedure was used to determine whether oscillatory activity differs between stimuli. T-tests were computed at each time point for each frequency band between the novel and dim conditions for the total power measure, resulting in a time-frequency t-map.

A permutation procedure was employed to estimate the probabilities of the values in the t-map, a procedure that is effective for multiple comparisons tests (Maris & Oostenveld, 2007). The resulting time-frequency map of p-values for novel vs. dim responses was assessed for significance using p values greater than 0.975 or less than 0.025, which corresponds to a Type I error rate of 0.05. These p-maps were summed across channels to create a spatial histogram of novelty effects (novel>dim or novel<dim effects). Time-frequency clusters in the histogram were thresholded at 5 channels (corresponding to a binomial probability of p<0.025) and 1 cycle duration at each frequency. The spatial distributions of the time-frequency clusters were visualized using topographic maps.

### Results

EEGs were collected from 10 healthy control subjects and data were analyzed using time-frequency clusters (FIG. 19A). The p-value threshold of the novel>dim total power map was increased to 0.988 to eliminate time-frequency clusters in the pre-stimulus baseline period. Statistical non-parametric mapping revealed 5 distinct time-frequency clusters that were significantly regulated in the subjects in response to presentation of the visual novelty oddball stimulus when compared to the dim stimulus. The first non-ERP component of the EEG that exhibited an increase in power was in the Gamma_{Hi} frequency band. Subsequent changes in power were observed in the Gamma_{Low} frequency band. These data indicate that significant increases in neural oscillatory power in the Gamma_{Hi} band occur after presentation of novel visual stimuli in the human cortex. A low-frequency cluster was observed that represented the ERP, a synchronous EEG phenomenon associated with acute exposure to oddball sensory stimuli.

To further define the region where increases in gamma oscillations occur, statistical maps of time-frequency analyses of EEG data were computed for each scalp EEG electrode. Power was determined by analyzing the change in power between the novel and dim stimulus presentations. Data from the two front-most scalp electrodes revealed a specific and significant increase in Gamma oscillation power. Increases in gamma oscillation power, including the Gamma_{Hi} frequency band, appeared to be restricted to the right, frontal cortex. The increase in Gamma_{Hi} power was also observed when EEGs were analyzed using a windowed periodogram approach, which was applied to the rodent data (FIG. 19B). Collectively, these results indicate significant increases in the Gamma_{Hi} frequency band occur in the human frontal cortex in response to perception of novel visual stimuli. These results are consistent with previous observations in the mouse in which significant increases in Gamma_{Hi} oscillations were observed when animals were placed into a novel environment.

### REFERENCES FOR EXAMPLE 9

Courchesne E, Hillyard S A, Galambos R (1975). Stimulus novelty, task relevance, and the visual evoked potential in man. Electroencephalogr Clin Neurophysiol 39:131-143.
Maris E, Oostenveld R (2007). Non-parametric statistical testing of EEG- and MEG-data. J Neurosci Meth 164:177-190.

### Example 10

### Spectral Analysis of Electroencephalographic (EEG) Oscillations in the Human Frontal Cortex in Subjects with Cognitive Deficit—Schizophrenia

### Introduction

To determine whether any aspects of neural oscillations in the human cortex were modulated in response to visual novelty presentation, experiments are performed to investigate the EEG signatures measured in schizophrenic human patients in response to environmental novelty. The experiments indicate that aspects of neural oscillations in the human cortex in schizophrenic subjects are modulated in response to visual novelty presentation.

### Methods

### Subjects

Subjects are schizophrenic individuals recruited for this study.

### Task

Subjects are seated in a comfortable chair in a darkened room and given a visual “novelty oddball” task. The stimuli are presented on a cathode ray tube computer monitor, situated 100 cm from the subject's nasion. Following Courchesne et al. (1975), there are 4 types of stimuli: targets (the letter “X”), standards (the letter “Y”), novels (complex, colored patterns), and “dims” (grey squares). Stimuli are approximately 3° X 3° of visual angle.

The task is divided into blocks of trials. Each block of trials consists of a number of targets, novels, dims, and standards. The interval between stimulus onsets is ˜set and each stimulus is presented for a set length of time. The subjects' task is to press a button on the response box when a target stimulus was presented.

### EEG Recording and Processing

The EEG is continuously recorded at 512 Hz sampling rate using a 72-channel Biosemi ActiveTwo system at standard scalp electrode sites. Electrodes are also placed at just below the left eye and at the outer canthi of the left and right eyes for deriving the vertical and horizontal electro-oculograms (EOGs), respectively.

Following data acquisition, the EEG is segmented into epochs from −500 to 1498 ms relative to stimulus onset. To ensure that the standard condition has a similar signal-to-noise ratio as the other conditions, 15 standard trials are selected at random from each block for analysis instead of the full number of standards. The epochs are analyzed for artifacts using a criterion of +/−90 μV for amplitude, or greater than 150 μV amplitude range, on any channel. Independent component analysis is applied to remove EOG and other artifacts (muscle artifacts, bad channels). The artifact-free epochs are re-referenced to the average reference. A subject's data are excluded from further analysis if following artifact correction/rejection, a subject does not have at least 67 artifact-free trials in each condition summed across blocks (i.e., 75% artifact-free trials in each condition).

Event related potentials (ERP) are computed for each condition by averaging the single-trial epochs. Event-related time-frequency measures (total power and phase locking factor) are computed using the Morlet wavelet transform. The range of frequencies analyzed are 4-100 Hz (1 Hz resolution).

### Statistical Analysis

A statistical non-parametric mapping procedure is used to determine whether oscillatory activity differs between stimuli. T-tests are computed at each time point for each frequency band between the novel and dim conditions for the total power measure, resulting in a time-frequency t-map.

A permutation procedure is employed to estimate the probabilities of the values in the t-map, a procedure that is effective for multiple comparisons tests (Maris & Oostenveld, 2007). The resulting time-frequency map of p-values for novel vs. dim responses is assessed for significance using p values greater than 0.975 or less than 0.025, which corresponds to a Type I error rate of 0.05. These p-maps are summed across channels to create a spatial histogram of novelty effects (novel>dim or novel<dim effects). Time-frequency clusters in the histogram are thresholded at 5 channels (corresponding to a binomial probability of p<0.025) and 1 cycle duration at each frequency. The spatial distributions of the time-frequency clusters are visualized using topographic maps.

### Results

EEGs are collected from the subjects and data are analyzed using time-frequency clusters. Results indicate an intermediate gamma recording as seen in the rodent model described herein. Collectively, these results indicate an intermediate level of gamma power in the frontal cortex of a schizophrenic human patient in response to perception of novel visual stimuli. These results are consistent with previous observations in the mouse and demonstrate a reduced ability to mount and sustain high power gamma and bimodal distribution.

### REFERENCES FOR EXAMPLE 10

Courchesne E, Hillyard S A, Galambos R (1975). Stimulus novelty, task relevance, and the visual evoked potential in man. Electroencephalogr Clin Neurophysiol 39:131-143.
Maris E, Oostenveld R (2007). Non-parametric statistical testing of EEG- and MEG-data. J Neurosci Meth 164:177-190.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

What is claimed is:

1. A method of identifying a candidate therapeutic agent for treatment of a cognitive deficit, the method comprising:
(a) administering a test agent to a test animal, wherein the test animal comprises a cognitive deficit, and the cognitive deficit is characterized by a distribution of the power of gamma oscillations recorded from a brain area during the cognitive task that substantially differs from a control distribution of the power of gamma oscillations recorded from the brain area of a control animal during the cognitive task;
(b) recording gamma oscillations from the brain area of the test animal while the test animal is engaged in the cognitive task;
(c) determining the distribution of the power of gamma oscillations in the test animal during the cognitive task; and
(d) comparing the determined distribution of the power of gamma oscillations of the test animal to the control distribution of the power of gamma oscillations, wherein a test agent that substantially reduces a difference between the distribution of the power of gamma oscillations in the test animal compared to the control distribution, is identified as a candidate therapeutic agent for treatment of the cognitive deficit.

2. The method of claim 1, wherein the gamma oscillations are Gamma_{Hi} oscillations.

3. The method of claim 2, wherein the Gamma_{Hi} oscillations are in a range of 65 Hz to 90 Hz.

4. The method of claim 1, wherein the cognitive deficit is associated with schizophrenia.

5. The method of any one of claims 1 to 4, wherein the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety.

6. The method of any one of claims 1 to 5, wherein the control distribution is a bimodal distribution.

7. The method of any one of claims 1 to 6, wherein the test animal is a rodent.

8. The method of claim 7, wherein the rodent is a rat or mouse.

9. The method of any one of claims 1 to 6, wherein the test animal is a primate.

10. The method of claim 9, wherein the primate is a non-human primate.

11. The method of claim 9, wherein the primate is a human.

12. The method of any one of claims 1 to 11, wherein the animal has a neurological disorder or condition or is a non-human animal model of such neurological disorder or condition.

13. The method of claim 12, wherein the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety.

14. The method of claim 12, wherein the neurological disorder or condition or non-human animal model of such neurological disorder or condition is chemically induced.

15. The method of claim 14, wherein the neurological disorder or condition or non-human animal model of such neurological disorder or condition is chemically induced with a drug that impairs glutamatergic function in the animal.

16. The method of claim 15, wherein the drug is selected from: phencyclidine (PCP), MK-801, and ketamine.

17. The method of claim 14, wherein the neurological disorder or condition or non-human animal model of such neurological disorder or condition is chemically induced with a drug that enhances dopaminergic function in the animal.

18. The method of claim 17, wherein the drug is selected from: apomorphine, D-amphetamine, and methamphetamine.

19. The method of claim 14, wherein the neurological disorder or condition or non-human animal model of such neurological disorder or condition is chemically induced with a hallucinogenic drug.

20. The method of claim 19, wherein the hallucinogenic drug is selected from: mescaline, lysergic acid diethylamide (LSD), and psilocybin.

21. The method of claim 14, wherein the neurological disorder or condition or non-human animal model of such neurological disorder or condition is chemically induced with a drug that impairs cholinergic function.

22. The method of claim 21, wherein the drug is scopolamine.

23. The method of any one of claims 1 to 22, wherein the neurological disorder or condition or non-human animal model of such neurological disorder or condition is a genetically induced.

24. The method of claim 23, wherein the animal is a calcineurin knock-out mouse (CNKO mouse).

25. The method of claim 24, wherein calcineurin is knocked-out postnatally in forebrain neurons of the animal.

26. The method of any one of claims 1 to 25, wherein the cognitive task is a novel object recognition task.

27. The method of any one of claims 1 to 25, wherein the cognitive task is a Delayed Non-Match-To-Position task.

28. The method of any one of claims 1 to 25, wherein the cognitive task is an alternating T-Maze.

29. The method of any one of claims 1 to 25, wherein the cognitive task is a Set Shifting task, an 8-arm radial maze task, 5 choice serial reaction time test, or an odor spanning task.

30. The method of any one of claims 1 to 25, wherein the cognitive task utilizes both attention and executive function of the animal.

31. The method of any one of claims 1 to 30, wherein the brain area is the prefrontal cortex.

32. The method of any one of claims 1 to 30, wherein the brain area is the striatum.

33. The method of any one of claims 1 to 30, wherein the brain area is the hippocampus.

34. The method of any one of claims 1 to 30, wherein the brain area is a midbrain dopaminergic area.

35. The method of claim 34, wherein the midbrain dopaminergic area is ventral tegmental area.

36. The method of any one of claims 1 to 35, wherein recording gamma oscillations in (b) comprises recording a single-unit activity (SUA) from the brain area.

37. The method of any one of claims 1 to 35, wherein recording gamma oscillations in (b) comprises recording an electrophysiological signal from an implanted electrode.

38. The method of any one of claims 1 to 37, wherein recording gamma oscillations in (b) comprises recording from a brain area comprising the frontal association cortex.

39. The method of any one of claims 1 to 8, wherein the animal is a mouse and the gamma oscillations are recorded from a region of brain that is within medial-lateral extent posterior to the olfactory bulb, anterior to M2 motor cortex, and superficial to orbital cortex.

40. The method of any one of claims 1-8 and 39, wherein the animal is a mouse and recording gamma oscillations comprises recording from a brain area having the coordinates: from Bregma +0.37 cm rostral, +0.07 cm lateral, −0.05 cm deep from the brain surface.

41. The method of any one of claims 1 to 35, wherein recording gamma oscillations in (b) comprises recording an electrophysiological signal from an external electrode.

42. The method of claim 41, wherein the external electrode is a scalp electrode.

43. The method of any one of claims 1 to 42, wherein the candidate therapeutic agent is a bimodal modulator of gamma oscillation.

44. A method of identifying a candidate therapeutic agent for treatment of a cognitive deficit, the method comprising:
(a) administering a test agent to a test animal, wherein the test animal is an animal comprising a cognitive deficit, and the cognitive deficit is characterized by a distribution of the power of electroencephalographic oscillations recorded from a brain area during a cognitive task that substantially differs from a control distribution of the power of electroencephalographic oscillations recorded from the brain area of a control animal during the cognitive task;
(b) recording electroencephalographic oscillations from the brain area of the test animal while the test animal is engaged in the cognitive task;
(c) determining the distribution of the power of electroencephalographic oscillations in the test animal during the cognitive task; and
(d) comparing the determined distribution of the power of electroencephalographic oscillations of the test animal to the control distribution of the power of electroencephalographic oscillations, wherein a test agent that substantially reduces a difference between the distribution of the power of electroencephalographic oscillations in the test animal compared to the control distribution, is identified as a candidate therapeutic agent for treatment of the cognitive deficit.

45. The method of claim 44, wherein the electroencephalographic oscillations are gamma oscillations.

46. The method of claim 45, wherein the gamma oscillations are Gamma_{Low} oscillations.

47. The method of claim 45, wherein the gamma oscillations are Gamma_{Hi} oscillations.

48. The method of claim 45, wherein the gamma oscillations are in a range of 30 Hz to 90 Hz.

49. The method of claim 46, wherein the Gamma_{Low} oscillations are in a range of 30 Hz to 55 Hz.

50. The method of claim 47, wherein the Gamma_{Hi} oscillations are in a range of 65 Hz to 90 Hz.

51. The method of claim 44, wherein the electroencephalographic oscillations are gamma oscillations that have an average power when recorded from a control animal exposed to a novel environment that is substantially higher than the average power when recorded from a control animal exposed to a familiar environment.

52. The method of claim 44, wherein the electroencephalographic oscillations are gamma oscillations that have an average power when recorded from a calcineurin knock out animal exposed to a novel environment that is substantially equal to the average power when recorded from a control animal exposed to a familiar environment.

53. The method of claim 44, wherein the electroencephalographic oscillations are theta oscillations or ripple oscillations.

54. The method of claim 53, wherein the theta oscillations are in a range of 4 Hz to 12 Hz.

55. The method of claim 53, wherein the ripple oscillations are in a range of 100 Hz to 300 Hz.

56. The method of any one of claims 44 to 55, wherein the cognitive deficit is associated with schizophrenia.

57. The method of any one of claims 44 to 55, wherein the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety.

58. The method of any one of claims 44 to 55, wherein the control distribution is a bimodal distribution.

59. The method of any one of claims 44 to 58, wherein the test animal is a rodent.

60. The method of claim 59, wherein the rodent is a rat or mouse.

61. The method of any one of claims 44 to 58, wherein the test animal is a primate.

62. The method of claim 61, wherein the primate is a non-human primate.

63. The method of claim 61, wherein the primate is a human.

64. The method of any one of claims 44 to 63, wherein the animal has a neurological disorder or condition or is a non-human animal model of such neurological disorder or condition.

65. The method of claim 64, wherein the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety.

66. The method of claim 64 or 65, wherein the neurological disorder or condition or non-human animal model of such neurological disorder or condition is chemically induced.

67. The method of claim 66, wherein the neurological disorder or condition or non-human animal model of such neurological disorder or condition is chemically induced with a drug that impairs glutamatergic function in the animal.

68. The method of claim 67, wherein the drug is selected from: phencyclidine (PCP), MK-801, and ketamine.

69. The method of claim 66, wherein the neurological disorder or condition or non-human animal model of such neurological disorder or condition is chemically induced with a drug that enhances dopaminergic function in the animal.

70. The method of claim 69, wherein the drug is selected from: apomorphine, D-amphetamine, and methamphetamine.

71. The method of claim 66, wherein the neurological disorder or condition or non-human animal model of such neurological disorder or condition is chemically induced with a hallucinogenic drug.

72. The method of claim 71, wherein the hallucinogenic drug is selected from: mescaline, lysergic acid diethylamide (LSD), and psilocybin.

73. The method of claim 66, wherein the neurological disorder or condition or non-human animal model of such neurological disorder or condition is chemically induced with a drug that impairs cholinergic function.

74. The method of claim 73, wherein the drug is scopolamine.

75. The method of any one of claims 44 to 65, wherein the neurological disorder or condition or non-human animal model of such neurological disorder or condition is genetically induced.

76. The method of claim 44-60, wherein the animal is a calcineurin knock-out mouse (CNKO mouse).

77. The method of claim 76, wherein calcineurin is knocked-out postnatally in forebrain neurons of the animal.

78. The method of any one of claims 44 to 77, wherein the cognitive task is a novel object recognition task.

79. The method of any one of claims 44 to 77, wherein the cognitive task is a Delayed Non-Match-To-Position task.

80. The method of any one of claims 44 to 77, wherein the cognitive task is an alternating T-Maze.

81. The method of any one of claims 44 to 77, wherein the cognitive task is a Set Shifting task, an 8-arm radial maze task, 5 choice serial reaction time test, or an odor spanning task.

82. The method of any one of claims 44 to 77, wherein the cognitive task utilizes both attention and executive function of the animal.

83. The method of any one of claims 44 to 82, wherein the brain area is the prefrontal cortex.

84. The method of any one of claims 44 to 82, wherein the brain area is the striatum.

85. The method of any one of claims 44 to 82, wherein the brain area is the hippocampus.

86. The method of any one of claims 44 to 82, wherein the brain area is a midbrain dopaminergic area.

87. The method of claim 86, wherein the midbrain dopaminergic area is ventral tegmental area.

88. The method of any one of claims 44 to 87, wherein recording electroencephalographic oscillations in (b) comprises recording a single-unit activity (SUA) from the brain area.

89. The method of any one of claims 44 to 87, wherein recording electroencephalographic oscillations in (b) comprises recording an electrophysiological signal from an implanted electrode.

90. The method of any one of claims 44 to 87, wherein recording electroencephalographic oscillations in (b) comprises recording from a brain area comprising the frontal association cortex.

91. The method of any one of claims 44 to 60, wherein the electroencephalographic oscillations are recorded from a region of brain that is within medial-lateral extent posterior to the olfactory bulb, anterior to M2 motor cortex, and superficial to orbital cortex.

92. The method of any one of claims 44 to 60, wherein the animal is a mouse and recording electroencephalographic oscillations comprises recording from a brain area having the coordinates: from Bregma +0.37 cm rostral, +0.07 cm lateral, −0.05 cm deep from the brain surface.

93. The method of any one of claims 44 to 87, wherein recording electroencephalographic oscillations in (b) comprises recording an electrophysiological signal from an external electrode.

94. The method of claim 85, wherein the external electrode is a scalp electrode.

95. The method of any one of claims 44 to 94, wherein the candidate therapeutic agent is a bimodal modulator of gamma oscillation.

96. A method of identifying a candidate therapeutic agent for treatment of a cognitive deficit, the method comprising:
(a) administering a test agent to a test animal, wherein the test animal is an animal comprising a cognitive deficit, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task
(b) recording an electroencephalographic oscillation from the brain area of the test animal while the test animal is engaged in the cognitive task; and
(c) comparing, in the predetermined frequency range, the recorded electroencephalographic oscillation of the test animal to the control electroencephalographic oscillation, wherein a test agent that substantially reduces a difference between the electroencephalographic oscillation in the test animal compared to the control electroencephalographic oscillation, is identified as a candidate therapeutic agent for treatment of the cognitive deficit.

97. The method of claim 96, wherein comparing in (c) comprises comparing power determined in the predetermined frequency range of the electroencephalographic oscillation of the test animal to power in the predetermined frequency range of the control electroencephalographic oscillation.

98. The method of claim 96 or 97, wherein comparing in (c) comprises comparing a distribution of powers of the electroencephalographic oscillation to a distribution of powers of the control electroencephalographic oscillation.

99. The method of claim 96 or 97, wherein comparing in (c) comprises comparing a frequency histogram of powers determined in predetermined time intervals of the electroencephalographic oscillation to a frequency histogram of powers determined in predetermined time intervals of the control electroencephalographic oscillation.

100. The method of any one of claims 96 to 99, wherein the predetermined frequency range is 30 Hz to 90 Hz.

101. The method of any one of claims 96 to 99, wherein the predetermined frequency range is 65 Hz to 90 Hz.

102. The method of any one of claims 96 to 99, wherein the predetermined frequency range is 30 Hz to 55 Hz.

103. The method of any one of claims 96 to 99 wherein the predetermined frequency range is a frequency range of a theta oscillation or a frequency range of a ripple oscillation.

104. The method of any one of claims 96 to 99, wherein the predetermined frequency range is a frequency range within which the electroencephalographic oscillation has an average power when recorded from a control animal exposed to a novel environment that is substantially higher than the average power when recorded from a control animal exposed to a familiar environment.

105. The method of any one of claims 96 to 99, wherein the predetermined frequency range is a frequency range within which the electroencephalographic oscillation has an average power when recorded from a calcineurin knock out animal exposed to a novel environment that is substantially equal to the average power when recorded from a control animal exposed to a familiar environment.

106. The method of any one of claims 96 to 105, wherein the cognitive deficit is associated with schizophrenia.

107. The method of any one of claims 96 to 105, wherein the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety.

108. The method of any one of claims 96 to 105, wherein the control distribution is a bimodal distribution.

109. The method of any one of claims 96 to 108, wherein the test animal is a rodent.

110. The method of claim 109, wherein the rodent is a rat or mouse.

111. The method of any one of claims 96 to 108, wherein the test animal is a primate.

112. The method of claim 111, wherein the primate is a non-human primate.

113. The method of claim 112, wherein the primate is a human.

114. The method of any one of claims 96 to 113, wherein the animal has a neurological disorder or condition or is a non-human animal model of such neurological disorder or condition.

115. The method of claim 114, wherein the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety.

116. The method of any one of claims 96 to 115, wherein the neurological disorder or condition or non-human animal model of such neurological disorder or condition is chemically induced.

117. The method of claim 116, wherein the neurological disorder or condition or non-human animal model of such neurological disorder or condition is chemically induced with a drug that impairs glutamatergic function in the animal.

118. The method of claim 117, wherein the drug is selected from: phencyclidine (PCP), MK-801, and ketamine.

119. The method of claim 116, wherein the neurological disorder or condition or non-human animal model of such neurological disorder or condition is chemically induced with a drug that enhances dopaminergic function in the animal.

120. The method of claim 119, wherein the drug is selected from: apomorphine, D-amphetamine, and methamphetamine.

121. The method of claim 116, wherein the neurological disorder or condition or non-human animal model of such neurological disorder or condition is chemically induced with a hallucinogenic drug.

122. The method of claim 121, wherein the hallucinogenic drug is selected from: mescaline, lysergic acid diethylamide (LSD), and psilocybin.

123. The method of claim 116, wherein the neurological disorder or condition or non-human animal model of such neurological disorder or condition is chemically induced with a drug that impairs cholinergic function.

124. The method of claim 123, wherein the drug is scopolamine.

125. The method of any one of claims 115 to 124, wherein the neurological disorder or condition or non-human animal model of such neurological disorder or condition is genetically induced.

126. The method of claim any one of claim 96-110 or 125, wherein the animal is a calcineurin knock-out mouse (CNKO mouse).

127. The method of claim 126, wherein calcineurin is knocked-out postnatally in forebrain neurons of the animal.

128. The method of any one of claims 96 to 127, wherein the cognitive task is a novel object recognition task.

129. The method of any one of claims 96 to 127, wherein the cognitive task is a Delayed Non-Match-To-Position task.

130. The method of any one of claims 96 to 127, wherein the cognitive task is an alternating T-Maze.

131. The method of any one of claims 96 to 127, wherein the cognitive task is a Set Shifting task, an 8-arm radial maze task, 5 choice serial reaction time test, or an odor spanning task.

132. The method of any one of claims 96 to 127, wherein the cognitive task utilizes both attention and executive function of the animal.

133. The method of any one of claims 96 to 132, wherein the brain area is the prefrontal cortex.

134. The method of any one of claims 96 to 132, wherein the brain area is the striatum.

135. The method of any one of claims 96 to 132, wherein the brain area is the hippocampus.

136. The method of any one of claims 96 to 132, wherein the brain area is a midbrain dopaminergic area.

137. The method of claim 136, wherein the midbrain dopaminergic area is ventral tegmental area.

138. The method of any one of claims 96 to 137, wherein recording electroencephalographic oscillations in (b) comprises recording a single-unit activity (SUA) from the brain area.

139. The method of any one of claims 96 to 137, wherein recording electroencephalographic oscillations in (b) comprises recording an electrophysiological signal from an implanted electrode.

140. The method of any one of claims 96 to 137, wherein recording electroencephalographic oscillations in (b) comprises recording from a brain area comprising the frontal association cortex.

141. The method of any one of claims 96 to 110, wherein the electroencephalographic oscillations are recorded from a region of brain that is within medial-lateral extent posterior to the olfactory bulb, anterior to M2 motor cortex, and superficial to orbital cortex.

142. The method of any one of claims 96 to 110, wherein the animal is a mouse and the recording electroencephalographic oscillations comprises recording from brain area having the coordinates: from Bregma +0.37 cm rostral, +0.07 cm lateral, −0.05 cm deep from the brain surface.

143. The method of any one of claims 96 to 137, wherein recording electroencephalographic oscillations in (b) comprises recording an electrophysiological signal from an external electrode.

144. The method of claim 143, wherein the external electrode is a scalp electrode.

145. The method of any one of claims 96 to 137, wherein the candidate therapeutic agent is a bimodal modulator of gamma oscillation.

146. A method of detecting a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, the method comprising:
(a) recording an electroencephalographic oscillation from the brain area of the animal while the animal is engaged in a cognitive task; and
(b) comparing, in the predetermined frequency range, the electroencephalographic oscillation recorded in (a) of the animal to the control electroencephalographic oscillation, wherein a substantial difference between the electroencephalographic oscillation in the animal compared to the control electroencephalographic oscillation, indicates that the animal has a cognitive deficit.

147. The method of claim 146, wherein a substantial difference between the electroencephalographic oscillation in the animal compared to the control electroencephalographic oscillation is detected and the method further comprises diagnosing the animal as having the cognitive deficit.

148. The method of claim 146 further comprising:
(c) determining a distribution of the power of gamma oscillations in the electroencephalographic oscillation recorded in (a); and
(d) obtaining a control distribution of the power of gamma oscillations in the control electroencephalographic oscillation,
wherein comparing in (b) comprises comparing, in the predetermined frequency range, the distribution of the power of gamma oscillations in the electroencephalographic oscillation determined in (c) to the distribution of the power of gamma oscillations in the control electroencephalographic oscillation obtained in (d),
wherein a substantial difference between the distribution of the power of gamma oscillations in the electroencephalographic oscillation determined in (c) compared to the distribution of the power of gamma oscillations in the control electroencephalographic oscillation obtained in (d), indicates that the animal has the cognitive deficit.

149. The method of claim 148, wherein a substantial difference between the distribution of the power of gamma oscillations in the electroencephalographic oscillation determined in (c) compared to the distribution of the power of gamma oscillations in the control electroencephalographic oscillation obtained in (d) is detected and the method further comprises diagnosing the animal as having the cognitive deficit.

150. A method of monitoring a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, the method comprising:
(a) recording an electroencephalographic oscillation from the brain area of the animal while the animal is engaged in the cognitive task;
(b) comparing, in the predetermined frequency range, the electroencephalographic oscillation recorded in (a) of the animal to the control electroencephalographic oscillation, wherein a substantial difference between the electroencephalographic oscillation in the animal compared to the control electroencephalographic oscillation, indicates that the animal has a cognitive deficit; and
(c) repeating steps (a) and (b) one or more times, thereby monitoring the cognitive deficit in the animal.

151. The method of claim 150, further comprising:
(d) administering a treatment for the cognitive disorder to the animal before (c), and
(e) comparing the electroencephalographic oscillation recorded in the animal before the treatment to the electroencephalographic oscillation recorded in the animal after the treatment to monitor the efficacy of the treatment.

152. A method of monitoring the effect of a treatment on a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, the method comprising:
(a) recording an electroencephalographic oscillation from the brain area of the animal with a cognitive deficit while the animal is engaged in the cognitive task;
(b) determining a distribution of the power of gamma oscillations in the electroencephalographic oscillation recorded in the animal;
(c) administering a treatment for the cognitive deficit or for a disease associated with the cognitive deficit to the animal with the cognitive impairment;
(d) recording an electroencephalographic oscillation from the brain area of the treated animal while the animal is engaged in the cognitive task;
(e) determining a distribution of the power of gamma oscillations in the electroencephalographic oscillation recorded in the treated animal; and
(f) comparing the distribution of power in (b) to the distribution of power in (e), wherein a substantial difference in the power in (b) and the power in (e) indicates an effect of the treatment on the cognitive deficit in the animal, and wherein a distribution of power in (e) that is more similar to a normal control distribution of power than is the distribution of power in (b), indicates efficacy of the treatment.

153. A method of determining the efficacy of a treatment for a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, the method comprising:
(a) recording an electroencephalographic oscillation from the brain area of the animal while the animal is engaged in the cognitive task;
(b) determining a distribution of the power of gamma oscillations in the electroencephalographic oscillation recorded in (a) of the animal;
(c) comparing, in the predetermined frequency range, the distribution of the power of gamma oscillations determined in (b) to a control distribution of the power of gamma oscillations, wherein a substantial difference between the distribution of the power of gamma oscillations in the animal compared to the control distribution, indicates that the animal has a cognitive deficit;
(d) administering a treatment for the cognitive deficit to the animal; and
(e) repeating steps (a) to (c) one or more times after administering the treatment in step (d), wherein a substantial decrease in a difference between the distribution of the power of gamma oscillations in the animal compared to the control distribution, indicates that the treatment is effective for treating the cognitive deficit.

154. The method of claim 150 or 151, wherein the treatment is a precognitive agent, an antipsychotic, antidepressant, anti-dementia, antiepileptic or anti-anxiety medication.

155. The method of any one of claims 146 to 154, wherein the predetermined frequency range is 30 Hz to 90 Hz.

156. The method of any one of claims 146 to 154, wherein the predetermined frequency range is 65 Hz to 90 Hz.

157. The method of any one of claims 146 to 154, wherein the predetermined frequency range is 30 Hz to 55 Hz.

158. The method of any one of claims 146 to 154, wherein the predetermined frequency range is a frequency range of a theta oscillation or a frequency range of a ripple oscillation.

159. The method of any one of claims 146 to 154, wherein the predetermined frequency range is a frequency range within which the electroencephalographic oscillation has an average power when recorded from a control animal exposed to a novel environment that is substantially higher than the average power when recorded from a control animal exposed to a familiar environment.

160. The method of any one of claims 146 to 154, wherein the predetermined frequency range is a frequency range within which the electroencephalographic oscillation has an average power when recorded from a calcineurin knock out animal exposed to a novel environment that is substantially equal to the average power when recorded from a control animal exposed to a familiar environment.

161. The method of any one of claims 146 to 160, wherein the cognitive deficit is associated with schizophrenia.

162. The method of any one of claims 146 to 160, wherein the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety.

163. The method of any one of claims 151 to 162, wherein the control distribution is a bimodal distribution.

164. The method of any one of claims 146, 148 and 150 to 163, wherein the animal is a rodent.

165. The method of claim 164, wherein the rodent is a rat or mouse.

166. The method of any one of claims 146 to 165, wherein the animal is a primate.

167. The method of claim 166, wherein the primate is a non-human primate.

168. The method of claim 166, wherein the primate is a human.

169. The method of any one of claims 146, 148 and 150 to 167, wherein the animal has a neurological disorder or condition or is a non-human animal model of such neurological disorder or condition.

170. The method of claim 169, wherein the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety.

171. The method of any one of claim 169 or 170, wherein the neurological disorder or condition or non-human animal model of such neurological disorder or condition is chemically induced.

172. The method of claim 171, wherein the neurological disorder or condition or non-human animal model of such neurological disorder or condition is chemically induced with a drug that impairs glutamatergic function in the animal.

173. The method of claim 172, wherein the drug is selected from: phencyclidine (PCP), MK-801, and ketamine.

174. The method of claim 171, wherein the neurological disorder or condition or non-human animal model of such neurological disorder or condition is chemically induced with a drug that enhances dopaminergic function in the animal.

175. The method of claim 174, wherein the drug is selected from: apomorphine, D-amphetamine, and methamphetamine.

176. The method of claim 171, wherein the neurological disorder or condition or non-human animal model of such neurological disorder or condition is chemically induced with a hallucinogenic drug.

177. The method of claim 176, wherein the hallucinogenic drug is selected from: mescaline, lysergic acid diethylamide (LSD), and psilocybin.

178. The method of claim 171, wherein the neurological disorder or condition or non-human animal model of such neurological disorder or condition is chemically induced with a drug that impairs cholinergic function.

179. The method of claim 178, wherein the drug is scopolamine.

180. The method of any one of claim 169 or 170, wherein the neurological disorder or condition or non-human animal model of such neurological disorder or condition is genetically induced.

181. The method of any one of claims 146, 148 and 150 to 165, wherein the animal is a calcineurin knock-out mouse (CNKO mouse).

182. The method of claim 181, wherein calcineurin is knocked-out postnatally in forebrain neurons of the animal.

183. The method of any one of claims 146 to 182, wherein the cognitive task is a novel object recognition task.

184. The method of any one of claims 146 to 182, wherein the cognitive task is a Delayed Non-Match-To-Position task.

185. The method of any one of claims 146 to 182, wherein the cognitive task is an alternating T-Maze.

186. The method of any one of claims 146 to 182, wherein the cognitive task is a Set Shifting task, an 8-arm radial maze task, 5 choice serial reaction time test, or an odor spanning task.

187. The method of any one of claims 146 to 182, wherein the cognitive task is a Novelty Oddball task.

188. The method of any one of claims 146 to 187, wherein the cognitive task utilizes both attention and executive function of the animal.

189. The method of any one of claims 146 to 188, wherein the brain area is the prefrontal cortex.

190. The method of any one of claims 146 to 189, wherein the brain area is the striatum.

191. The method of any one of claims 146 to 190, wherein the brain area is the hippocampus.

192. The method of any one of claims 146 to 191, wherein the brain area is a midbrain dopaminergic area.

193. The method of claim 192, wherein the midbrain dopaminergic area is ventral tegmental area.

194. The method of any one of claims 146 to 193, wherein recording electroencephalographic oscillations in (b) comprises recording a single-unit activity (SUA) from the brain area.

195. The method of any one of claims 146 to 193, wherein recording electroencephalographic oscillations in (b) comprises recording an electrophysiological signal from an implanted electrode.

196. The method of any one of claims 146 to 195, wherein recording electroencephalographic oscillations in (b) comprises recording from a brain area comprising the frontal association cortex.

197. The method of any one of claims 146 to 165, wherein the electroencephalographic oscillations are recorded from a region of brain that is within medial-lateral extent posterior to the olfactory bulb, anterior to M2 motor cortex, and superficial to orbital cortex.

198. The method of any one of claims 146 to 165, wherein the animal is a mouse and the recording electroencephalographic oscillations comprises recording from brain area having the coordinates: from Bregma +0.37 cm rostral, +0.07 cm lateral, −0.05 cm deep from the brain surface.

199. The method of any one of claims 146 to 195, wherein recording electroencephalographic oscillations in (b) comprises recording an electrophysiological signal from an external electrode.

200. The method of claim 199, wherein the external electrode is a scalp electrode.

201. A method of determining an effect of a candidate agent on a cognitive deficit in an animal, wherein the cognitive deficit is characterized by an electroencephalographic oscillation, recorded from a brain area during a cognitive task, that substantially differs, in a predetermined frequency range, from a control electroencephalographic oscillation recorded from the brain area of a control animal during the cognitive task, the method comprising:
(a) recording an electroencephalographic oscillation from the brain area of the animal with a cognitive deficit while the animal is engaged in the cognitive task;
(b) determining a distribution of the power of gamma oscillations in the electroencephalographic oscillation recorded in the animal;
(c) administering the candidate agent to the animal with the cognitive impairment;
(d) recording an electroencephalographic oscillation from the brain area of the treated animal while the animal is engaged in the cognitive task;
(e) determining a distribution of the power of gamma oscillations in the electroencephalographic oscillation recorded in the treated animal; and
(f) comparing the distribution of power in (b) to the distribution of power in (e), wherein a substantial difference in the distribution of power in (b) and the distribution of power in (e) indicates an effect of the candidate agent on the cognitive deficit in the animal.

202. The method of claim 201, wherein the candidate agent is a precognitive agent, an antipsychotic, antidepressant, anti-dementia, antiepileptic or anti-anxiety medication.

203. The method of claim 201, wherein the candidate agent is a small molecule.

204. The method of any one of claims 201 to 203, wherein the predetermined frequency range is 30 Hz to 90 Hz, 65 Hz to 90 Hz, or 30 Hz to 55 Hz.

205. The method of claim 201 or 202, wherein the method is utilized in a clinical trial.

206. The method of claim 201 or 202, wherein the distribution of the power of gamma oscillations is utilized as a biomarker in a clinical trial.

207. The method of any one of claims 201 to 206, wherein the predetermined frequency range is a frequency range of a theta oscillation or a frequency range of a ripple oscillation.

208. The method of any one of claims 201 to 207, wherein the predetermined frequency range is a frequency range within which the electroencephalographic oscillation has an average power when recorded from a control animal exposed to a novel environment that is substantially higher than the average power when recorded from a control animal exposed to a familiar environment.

209. The method of any one of claims 201 to 208, wherein the cognitive deficit is associated with schizophrenia.

210. The method of any one of claims 201 to 209, wherein the cognitive deficit is associated with psychosis, bipolar disorder, Alzheimer's disease, Parkinson's disease, Huntington's Disease, multiple sclerosis, Attention Deficit Hyperactivity Disorder (ADHD), autism, a learning disorder, an injury, or anxiety.

211. The method of any one of claims 201 to 210, wherein the control distribution is a bimodal distribution.

212. The method of claim 201, wherein the animal is a rodent.

213. The method of claim 212, wherein the rodent is a rat or mouse.

214. The method of claim 201, wherein the animal is a primate.

215. The method of claim 214, wherein the primate is a non-human primate.

216. The method of claim 215, wherein the primate is a human.

217. The method of any one of claims 201-216, wherein the animal has a neurological disorder or condition or is anon-human animal model of such neurological disorder or condition.

218. The method of claim 217, wherein the neurological disorder or condition is Schizophrenia, bipolar disorder, Alzheimer's disease, Parkinson's disease, Huntington's Disease, multiple sclerosis, Attention Deficit Hyperactivity Disorder (ADHD), autism, a learning disorder, an injury or anxiety.

219. The method of any one of claims 217, wherein the neurological disorder or condition or non-human animal model of such neurological disorder or condition is chemically induced.

220. The method of claim 217, wherein the neurological disorder or condition or non-human animal model of such neurological disorder or condition is chemically induced with a drug that impairs glutamatergic function in the animal.

221. The method of claim 220, wherein the drug is selected from: phencyclidine (PCP), MK-801, and ketamine.

222. The method of claim 217, wherein the neurological disorder or condition or non-human animal model of such neurological disorder or condition is chemically induced with a drug that enhances dopaminergic function in the animal.

223. The method of claim 222, wherein the drug is selected from: apomorphine, D-amphetamine, and methamphetamine.

224. The method of claim 217, wherein the neurological disorder or condition or non-human animal model of such neurological disorder or condition is chemically induced with a hallucinogenic drug.

225. The method of claim 224, wherein the hallucinogenic drug is selected from: mescaline, lysergic acid diethylamide (LSD), and psilocybin.

226. The method of claim 217, wherein the neurological disorder or condition or non-human animal model of such neurological disorder or condition is chemically induced with a drug that impairs cholinergic function.

227. The method of claim 226, wherein the drug is scopolamine.

228. The method of claim 217, wherein the neurological disorder or condition or non-human animal model of such neurological disorder or condition is genetically induced.

229. The method of claim 201, wherein the animal is a calcineurin knock-out mouse (CNKO mouse).

230. The method of claim 229, wherein calcineurin is knocked-out postnatally in forebrain neurons of the animal.

231. The method of any one of claims 201-230, wherein the cognitive task is a novel object recognition task, a Delayed Non-Match-To-Position task, an alternating T-Maze, a Set Shifting task, an 8-arm radial maze task, 5 choice serial reaction time test, or an odor spanning task, or a Novelty Oddball task.

232. The method of any one of claims 201-231, wherein the cognitive task utilizes both attention and executive function of the animal.

233. The method of any one of claims 200-232, wherein the brain area is the prefrontal cortex.

234. The method of any one of claims 200-233, wherein the brain area is the striatum.

235. The method of any one of claims 200-234, wherein the brain area is the hippocampus.

236. The method of claim 201, wherein the brain area is a midbrain dopaminergic area.

237. The method of claim 236, wherein the midbrain dopaminergic area is ventral tegmental area.

238. The method of any one of claims 201-237, wherein recording electroencephalographic oscillations in (b) comprises recording a single-unit activity (SUA) from the brain area.

239. The method of any one of claims 201-237, wherein recording electroencephalographic oscillations in (b) comprises recording an electrophysiological signal from an implanted electrode.

240. The method of any one of claims 201-237, wherein recording electroencephalographic oscillations in (b) comprises recording from a brain area comprising the frontal association cortex.

241. The method of any one of claims 201-237, wherein recording electroencephalographic oscillations in (b) comprises recording an electrophysiological signal from an external electrode.

242. The method of claim 241, wherein the external electrode is a scalp electrode.
